(19) United States
(12) Patent Application Publication
(10) Pub. No.: US 2026/0028621 A1
(43) Pub. Date: Jan. 29, 2026

(54) METHOD FOR ENCODING DIGITAL DATA ON NUCLEIC ACIDS USING BIOLOGICAL PROCESSES

(71) Applicants: CENTRE NATIONAL DE LA RECHERCHE SCIENTIFIQUE, Paris (FR); SORBONNE UNIVERSITE, Paris (FR)

(72) Inventors: Stéphane LEMAIRE, Paris (FR); Pierre CROZET, Paris (FR); Clémence BLACHON, Paris (FR); Nicolas CORNILLE, Paris (FR); Mariette GIBIER, Paris (FR); Achille JULIENNE, Paris (FR)

(73) Assignees: CENTRE NATIONAL DE LA RECHERCHE SCIENTIFIQUE, Paris (FR); SORBONNE UNIVERSITE, Paris (FR)

(21) Appl. No.: 18/867,295

(22) PCT Filed: May 19, 2022
(86) PCT No.: PCT/IB2022/000298

§ 371 Date: Nov. 19, 2024

(51) Int. Cl. C12N 15/10; G06N 3/123

(52) U.S. Cl. CPC C12N 15/1093; G06N 3/123

(57) ABSTRACT

A nucleic acid-based data storage method for storing information, and to a data storage nucleic acid molecule.

### FIELD

The present invention relates to nucleic acid-based data storage methods for storing digital information.

### BACKGROUND

Storing and archiving digital data are major issues in our modern societies. The current digital media stored in data centers are fragile, bulky and energy-consuming. Although optical media, magnetic tapes, hard drives or flash memory have been developed, their durability does not exceed ten years on average. These data must be regularly copied onto new reliable media and have to be maintained at controlled temperature and humidity, inducing a colossal energy cost and requiring huge amounts of raw materials. The amount of energy consumed by data centers corresponds to 2% of the worldwide electricity consumption (Masanet et al. 2020). The carbon footprint of the data centers exceeds that of global civil aviation. Despite their energy cost, their carbon footprint and their increasing need for bulky area, data centers can only store 30% of the data we produce while our data production grows exponentially: “If today we are capable of storing about 30% of the information we generate, in only 10 or 12 years we will be able to store about 3%” (Dr. Karin Strauss, Microsoft Research, 2018). Given these general considerations, the data revolution, the big data market and the development of artificial intelligence cannot be pursued without finding innovative solutions to the problem of data storage.

US2018/0137418 describes the use of chemically produced DNA bricks and assembles several of them (3-6) to make a larger molecule (a few hundred base pairs) to encode the information bit (0 or 1). However, these processes are time consuming and costly.

Consequently, there is still a need for new means for storing digital data that can sustain encoding of large amounts of data, and can further be biocompatible, i.e., that can be copied, edited, written and/or read using living organisms.

### SUMMARY

The present invention relates to a nucleic acid-based data storage method for storing information comprising:
a) recovering data in the form of a digital sequence formed of a plurality of bits, each bit having the value 0 or 1,
b) subdividing the digital sequence into n digital subsequences, each comprising m bits, m being comprised between 2 and 16,
c) converting each of the n digital subsequences into a bioblock, a bioblock consisting of a sequence of m nucleotides,
   wherein the digital subsequence consists in m bits assigned to positions 0 to m−1, and
   wherein the conversion of a digital subsequence into a bioblock consists in:
   converting bits at even positions to a first nucleotide N1 if said bits has the value 0, and to a second distinct nucleotide N2 if said bits has the value 1 and
   converting bits at odd positions to a third nucleotide N3 if said bits has the value 0, and to a fourth distinct nucleotide N4 if said bits has the value 1,
   wherein N1, N2, N3 and N4 are distinct nucleotides
d) constructing a plurality of x components, each individual component of the plurality of x components comprising at least one bioblock, and the x components together comprising n bioblocks
e) assembling together in a fixed order, in one or more steps, the plurality of x components.

In some embodiments, the nucleotides are selected from the group of natural nucleotides consisting of adenine, guanine, cytosine, uracil and thymine or from non-natural nucleotides.

In some embodiments, the x components are x DNA molecules, preferably x double-stranded DNA molecules.

In some embodiments, at step (d) the construction of a plurality of x components, each comprising at least one bioblock, comprises the steps of:
selectively capturing x data storage nucleic acid molecules from at least one library of data storage nucleic acid molecules, wherein each data storage nucleic acid molecule comprises at least one bioblock surrounded by regions comprising cleavage sites,
cleaving each of the x data storage nucleic acid molecules, thereby releasing the at least one bioblock.

In some embodiments, at step (d) the construction of a plurality of x components, each comprising at least one bioblock, comprises the steps of:
selectively capturing n data storage nucleic acid molecules from at least two libraries of data storage nucleic acid molecules, wherein each data storage nucleic acid molecule of each library comprises one bioblock surrounded by regions comprising cleavage sites, and wherein each library comprises all possible bioblocks of m nucleotides,
cleaving each of the n data storage nucleic acid molecules, thereby releasing the n bioblocks.

In some embodiments, the regions comprising cleavage sites comprises from 2 to 25 nucleotides.

In some embodiments, the region surrounding each bioblock comprises a site for a restriction enzyme, and step (d) comprises a step of digesting each of the x data storage nucleic acid molecules with one or two restriction enzymes.

In some embodiments, step (e) comprises one or several assembling steps using overlap-extension polymerase chain reaction (PCR), polymerase cycling assembly, sticky end ligation, biobricks assembly, golden gate assembly, Gibson assembly, recombinase assembly, ligase cycling reaction, template directed ligation, in vivo assembly or any other DNA assembly protocol.

The present invention further relates to a data storage nucleic acid molecule comprising at least one bioblock, a bioblock consisting of a nucleic acid sequence consisting of m nucleotides assigned to positions 0 to m−1, wherein
a bioblock is formed of at least 2 and at most 4 distinct nucleotides
nucleotides at even positions may be selected from a first and a second nucleotide, and nucleotides at odd positions may be selected from a third and a fourth nucleotide, said first, second, third and fourth nucleotides being distinct.

In some embodiments, the data storage nucleic acid molecule is a double-stranded molecule, preferably a DNA molecule.

In some embodiments, the data storage nucleic acid molecule is a plasmid, a cosmid, a fosmid, a prokaryotic chromosome or a eukaryotic chromosome.

In some embodiments, each of the bioblock is surrounded by regions comprising cleavage sites, preferably by two sites for one restriction enzyme.

In some embodiments, the data storage nucleic acid molecule is replicative.

The present invention further relates to a library comprising a plurality of data storage nucleic acid molecules according to the invention, wherein each of the data storage nucleic acid molecule of the library contains one bioblock, wherein each data storage nucleic acid molecule of the library comprises the same surrounding regions comprising cleavage sites and wherein the library contains all possible bioblocks of m nucleotides.

The present invention further relates to a nucleic acid-based data storage system comprising at least two libraries according to the invention.

### Definitions

In the present invention, the following terms have the following meanings:

The term “digital data” refers to data that can be managed by computerized machines. As used herein, the expression “digital data” is meant to refer to data represented by a binary system. As used herein, a “binary system” refers to a language composed of bits “0” and “1”. Non-limitative examples of digital data may be program files, text files, music files, image files, video files and combinations thereof.

The term “storage” or “storing” refers to the action of keeping an item in a specific place for future use or for safekeeping. More specifically, the expression “storage of digital data” is intended to mean the action of safely keeping the digital information for further use.

The term “replicative” refers to the ability to be replicated in vivo by a polymerase, such as, e.g., a DNA polymerase, i.e., to be exactly duplicated, within the margin of error of replication mechanisms of living organisms. As used herein, a “replicative nucleic acid molecule” is intended to refer to a nucleic acid molecule that can be copied at least once in vivo. In one embodiment, the nucleic acid molecule according to the invention is selected in the group consisting of a plasmid, a cosmid and a chromosome. In practice, a replicative nucleic acid molecule comprises one or more origin(s) of replication (also termed ORI), or one or more centromere(s) (for chromosomes).

Within the scope of the present invention, the term “nucleotide” and “nucleic base” are meant as substitutes for one another and are intended to refer to the nucleic building block of a DNA or RNA molecule. Nucleotides comprise both natural nucleotides and non-natural nucleotides. As used herein, a natural nucleotide refers to a purine Adenine (A) or Guanine (G); or to a pyrimidine Cytosine (C), Thymine (T) or Uracil (U). For DNA nucleic acids, A refers to the dAMP deoxyribonucleotide; G refers to the dGMP deoxyribonucleotide; C refers to the dCMP deoxyribonucleotide; and T refers to the dTMP deoxyribonucleotide. For RNA nucleic acids, A refers to the AMP ribonucleotide; G refers to the GMP ribonucleotide; C refers to the CMP ribonucleotide; and U refers to the UMP ribonucleotide. As used herein, the term “non-natural nucleotides” refers to chemically modified A, T, U, C or G nucleotides. Non limitative examples of non-natural nucleotides include 2-Amino-ATP, 8-Aza-ATP, 2′-Fluoro-dATP, 2′-Fluoro-dCTP, 2′-Fluoro-dGTP, 2′-Fluoro-dUTP, 5-Iodo-CTP, 5-Iodo-UTP, N6-Methyl-ATP, 5-Methyl-CTP, 2′-O-Methyl-ATP, 2′-O-Methyl-CTP, 2′-O-Methyl-GTP, 2′-O-Methyl-UTP, Pseudo-UTP, ITP, 2′-O-Methyl-ITP, Puromycin-TP, Xanthosine-TP, 5-Methyl-UTP, 4-Thio-UTP, 2′-Amino-dCTP, 2′-Amino-dUTP, 2′-Azido-dCTP, 2′-Azido-dUTP, 06-Methyl-GTP, 2-Thio-UTP, Ara-CTP, Ara-UTP, 5,6-Dihydro-UTP, 2-Thio-CTP, 6-Aza-CTP, 6-Aza-UTP, N1-Methyl-GTP, 2′-O-Methyl-2-Amino-ATP, 2′-O-Methylpseudo-UTP, N1-Methyl-ATP, 2′-O-Methyl-5-methyl-UTP, 7-Deaza-GTP, 2′-Azido-dATP, 2′-Amino-dATP, Ara-ATP, 8-Azido-ATP, 5-Bromo-CTP, 5-Bromo-UTP, 2′-Fluoro-dTTP, 3′-O-Methyl-ATP, 3′-O-Methyl-CTP, 3′-O-Methyl-GTP, 3′-O-Methyl-UTP, 7-Deaza-ATP, 5-AA-UTP, 2′-Azido-dGTP, 2′-Amino-dGTP, 5-AA-CTP, 8-Oxo-GTP, Pseudoiso-CTP, N4-Methyl-CTP, N1-Methylpseudo-UTP, 5,6-Dihydro-5-Methyl-UTP, N6-Methyl-Amino-ATP, 5-Carboxy-CTP, 5-Formyl-CTP, 5-Hydroxymethyl-UTP, 5-Hydroxymethyl-CTP, Thieno-GTP, 5-Hydroxy-CTP, 5-Formyl-UTP, Thieno-UTP, 2-Amino-dATP, 5-Bromo-dCTP, 5-Bromo-dUTP, 7-Deaza-dATP, 7-Deaza-dGTP, dITP, 5-Propynyl-dCTP, 5-Propynyl-dUTP, 2′-dUTP, 5-Fluoro-dUTP, 5-Iodo-dCTP, 5-Iodo-dUTP, N6-Methyl-dATP, 5-Methyl-dCTP, 06-Methyl-dGTP, N2-Methyl-dGTP, 8-Oxo-dATP, 8-Oxo-dGTP, 2-Thio-dTTP, 2′-dPTP, 5-Hydroxy-dCTP, 4-Thio-dTTP, 2-Thio-dCTP, 6-Aza-dUTP, 6-Thio-dGTP, 8-Chloro-dATP, 5-AA-dCTP, 5-AA-dUTP, N4-Methyl-dCTP, 2′-deoxyzebularine-TP, 5-Hydroxymethyl-dUTP, 5-Hydroxymethyl-dCTP, 5-Propargylamino-dCTP, 5-Propargylamino-dUTP, 5-Carboxy-dCTP, 5-Formyl-dCTP, 5-Indolyl-AA-dUTP, 5-Carboxy-dUTP, 5-Formyl-dUTP, 3′-dATP, 3′-dGTP, 3′-dCTP, 5-Methyl-3′-dUTP, 3′-dUTP, ddATP, ddGTP, ddUTP, ddTTP, ddCTP, 3′-Azido-ddATP, 3′-Azido-ddGTP, 3′-Azido-ddTTP, 3′-Amino-ddATP, 3′-Amino-ddCTP, 3′-Amino-ddGTP, 3′-Amino-ddTTP, 3′-Azido-ddCTP, 3′-Azido-ddUTP, 5-Bromo-ddUTP, ddITP, (1-Thio)-dATP, (1-Thio)-dCTP, (1-Thio)-dGTP, (1-Thio)-dTTP, (1-Thio)-ATP, (1-Thio)-CTP, (1-Thio)-GTP, (1-Thio)-UTP, (1-Thio)-ddATP, (1-Thio)-ddCTP, (1-Thio)-ddGTP, (1-Thio)-ddTTP, (1-Thio)-3′-Azido-ddTTP, (1-Thio)-ddUTP, (1-Borano)-dATP, (1-Borano)-dCTP, (1-Borano)-dGTP, (1-Borano)-dTTP, Ganciclovir-TP, Cidofovir-DP, 3-methyl-6-amino-5-(1′-b-D-2′-deoxyribofuranosyl)-pyrimidin-2-one, 6-amino-9[(1′-b-D-2′-deoxyribofuranosyl)-4-hydroxy-5-(hydroxymethyl)-oxolan-2-yl]-1H-purin-2-one, 6-amino-3-(1′-b-D-2′-deoxyribofuranosyl)-5-nitro-1H-pyridin-2-one and 2-amino-8-(1′-b-D-2′-deoxyribofuranosyl)-imidazo-[1,2a]-1,3,5-triazin-[8H]-4-one.

### DETAILED DESCRIPTION

The present invention relates to a nucleic acid-based data storage method for storing information comprising:
(a) recovering data in the form of a digital sequence formed of a plurality of bits, each bit having the value 0 or 1,
(b) subdividing the digital sequence into n digital subsequences, each comprising m bits, m being comprised between 2 and 16,
(c) converting each of the n digital subsequences into a bioblock, a bioblock consisting of a sequence of m nucleotides,
   wherein the digital subsequence consists in m bits assigned to positions 0 to m−1, and
   wherein the conversion of a digital subsequence into a bioblock consists in:
      converting bits at even positions to a first nucleotide N1 if said bits has the value 0, and to a second distinct nucleotide N2 if said bits has the value 1 and
      converting bits at odd positions to a third nucleotide N3 if said bits has the value 0, and to a fourth distinct nucleotide N4 if said bits has the value 1,
      wherein N1, N2, N3 and N4 are distinct nucleotides
(d) constructing a plurality of x components, each individual component of the plurality of x components comprising at least one bioblock, and the x components together comprising n bioblocks
(e) assembling together in a fixed order, in one or more steps, the plurality of x components.

As used herein, the term “bit” (binary digit) refers to the smallest base unit of digital information. In practice, a bit relies on a base-2 numeral system and can have the value of either 0 or 1. Methods to store bits involve the use of electronic devices and are well known in the art.

Within the scope of the present invention, the term “byte”, interchangeable with the terms “bit string” or “bit chain”, refers to a contiguous sequence of bits, herein also referred to as a “digital subsequence”. Within the scope of the present invention, the number of bits per byte corresponds to the value of m.

In one embodiment, the value of m is comprised between 2 and 16. As used herein, the term “between 2 and 16” means 2, 3, 4, 5, 6, 7, 8, 9, 10, 11, 12, 13, 14, 15 and 16. In one embodiment, the value of m is selected from the group comprising or consisting of 2, 4, 6, 8, 10, 12, 14 and 16. In one embodiment, the value of m is selected from the group comprising or consisting of 2, 4, 8 and 16.

In one embodiment, the value of m is 8. In practice, a byte consisting of 8 bits is referred herein as an octet; and a bioblock resulting from the conversion of an octet is referred herein as a biooctet.

In one embodiment, the value of m is 16. In one embodiment, the value of m is 4. In one embodiment, the value of m is 2.

In one embodiment, the digital sequence may be comprised in, or consist of, any digital file stored on a computer. In one embodiment, the file is a file type selected from the group comprising .3dm (Rhino 3D Model), .3ds (3D Studio Scene), .3g2 (3GPP2 multimedia file), .3gp (3GPP multimedia File), .accdb (Access 2007 Database file), .ai (Adobe Illustrator file), .aif (AIF/Audio Interchange audio file), .apk (Android package file), .asp and .aspx (Active Server Page file), .avi (Audio Video Interleave file), .bak (Backup file), .bat (Batch file), .bin (Binary file), .bmp (Bitmap image file), .cab (Windows Cabinet file), .cda (CD audio track file), .cer (Internet security certificate), .cfg (Configuration file), .cfm (ColdFusion Markup file), .cgi (Common Gateway Interface Script), .cgi or .pl (Perl script file), .com (MS-DOS command file), .cpl (Windows Control panel file), .css (Cascading Style Sheet file), .csv (Comma separated value file), .cur (Windows cursor file), .dat (Data file), .db or .dbf (Database file), .dll (DLL file), .dmp (Dump file), .doc and .docx (Microsoft Word file), .drv (Device driver file), .exe (Executable file), .flv (Adobe Flash Video file), .gif (GIF/Graphical Interchange Format image), .h264 (H.264 video file), .htm and .html (HTML/Hypertext Markup Language file), .icns (macOS X icon resource file), .ico (Icon file), .ico (Icon file), .iff (Interchange File Format), .ini (Initialization file), .jar (Java Archive file), .jpeg or .jpg (JPEG image), .js (JavaScript file), .jsp (Java Server Page file), .key (Keynote presentation), .lnk (Windows shortcut file), .log (Log file), .m4v (Apple MP4 video file), .max (3ds Max Scene file), .mdb (Microsoft Access database file), .mid or .midi (MIDI audio file), .mkv (Matroska Multimedia Container), .mov (Apple QuickTime movie file), .mp3 (MP3 audio file), .mp4 (MPEG-4 Video File), .mpa (MPEG-2 audio file), .mpg or .mpeg (MPEG video file), .msg (Outlook Mail Message), .msi (Windows installer package), .obj (Wavefront 3D Object file), .odp (OpenOffice Impress presentation file), .ods (OpenOffice Calc spreadsheet file), .odt (OpenOffice Writer document file), .part (Partially downloaded file), .pdb (Program Database), .pdf (PDF file), .php (PHP Source Code file), .png (PNG/Portable Network Graphic image), .pps (PowerPoint slide show), .ppt (PowerPoint presentation), .pptx (PowerPoint Open XML presentation), .ps (PostScript file), .psd (PSD/Adobe Photoshop Document image), .py (Python file), .rm (Real Media file), .rss (RSS/Rich Site Summary file), .rtf (Rich Text Format file), .sav (Save file), .sql (SQL/Structured Query Language database file), .svg (Scalable Vector Graphics file), .swf (Small Web Format file, formerly ShockWave Flash file), .sys (Windows system file), .tar (Linux/Unix tarball file archive), .tex (TeX document file), .tif or .tiff (TIFF image), .tmp (Temporary file), .txt (Plain text file), .vob (DVD Video Object file), .wav (WAVE file), .wks and .wps (Microsoft Works Word Processor Document file), .wma (Windows Media audio file), .wmv (Windows Media Video file), .wpd (WordPerfect document), .wpl (Windows Media Player playlist), .wsf (Windows Script File), .xhtml (XHTML/Extensible Hypertext Markup Language file), .xlr (Microsoft Works spreadsheet file), .xls (Microsoft Excel file), .xlsx (Microsoft Excel Open XML spreadsheet file).

In one embodiment, the digital sequence may be selected in a group comprising program files, text files, table files, audio files, image files, video files and combinations thereof.

In one embodiment, the digital sequence may be comprised in, or consist of, program files. Non-limitative examples of program files include .accdb (Access 2007 Database File), .apk (Android package file), .bak (Backup file), .bat (Batch file), .bin (Binary file), .cab (Windows Cabinet file), .cfg (Configuration file), .cgi (Common Gateway Interface Script), .com (MS-DOS command file), .cpl (Windows Control panel file), .csv (Comma separated value file), .cur (Windows cursor file), .dat (Data file), .db or .dbf (Database file), .dll (DLL file), .dmp (Dump file), .drv (Device driver file), .exe (Executable file), .icns (macOS X icon resource file), .ico (Icon file), .ini (Initialization file), .jar (Java Archive file), .lnk (Windows shortcut file), .log (Log file), .mdb (Microsoft Access database file), .msi (Windows installer package), .pdb (Program Database), .py (Python file), .sav (Save file), .sql (SQL/Structured Query Language database file), .sys (Windows system file), .tar (Linux/Unix tarball file archive), .tmp (Temporary file) and .wsf (Windows Script File).

In one embodiment, the digital sequence may be comprised in, or consist of, text files. Non-limitative examples of text files include .doc and .docx (Microsoft Word file), .odt (OpenOffice Writer document file), .msg (Outlook Mail Message), .pdf (PDF file), .rtf (Rich Text Format file), .tex (TeX document file), .txt (Plain text file), .wks and .wps (Microsoft Works Word Processor Document file), and .wpd (WordPerfect document).

In one embodiment, the digital sequence may be comprised in, or consist of, table files, e.g., spreadsheets. Non-limitative examples of table files include .ods (OpenOffice Calc spreadsheet file), .xlr (Microsoft Works spreadsheet file), .xls (Microsoft Excel file) and .xlsx (Microsoft Excel Open XML spreadsheet file).

In one embodiment, the digital sequence may be comprised in, or consist of, audio files, e.g., music files. Non-limitative examples of audio files include .aif (AIF/Audio Interchange audio file), .cda (CD audio track file), .iff (Interchange File Format), .mid or .midi (MIDI audio file), .mp3 (MP3 audio file), .mpa (MPEG-2 audio file), .wav (WAVE file), .wma (Windows Media audio file), and .wpl (Windows Media Player playlist).

In one embodiment, the digital sequence may be comprised in, or consist of, image files. Non-limitative examples of image files include .ai (Adobe Illustrator file), .bmp (Bitmap image file), .gif (GIF/Graphical Interchange Format image), .ico (Icon file), .jpeg or .jpg (JPEG image), .max (3ds Max Scene file), .obj (Wavefront 3D Object file), .png (PNG/Portable Network Graphic image), .ps (PostScript file), .eps (Encapsulated PostScript file), .psd (PSD/Adobe Photoshop Document image), .svg (Scalable Vector Graphics file), .tif or .tiff (TIFF image), .3ds (3D Studio Scene), and .3dm (Rhino 3D Model).

In one embodiment, the digital sequence may be comprised in, or consist of, video files. Non-limitative examples of video files include .avi (Audio Video Interleave File), .flv (Adobe Flash Video File), .h264 (H.264 video File), .m4v (Apple MP4 video File), .mkv (Matroska Multimedia Container), .mov (Apple QuickTime movie File), .mp4 (MPEG-4 Video File), .mpg or .mpeg (MPEG video File), .rm (Real Media File), .swf (Shockwave flash File), .vob (DVD Video Object File), .wmv (Windows Media Video File), .3g2 (3GPP2 Multimedia File), and .3gp (3GPP multimedia File).

In one embodiment, the total number of bytes, i.e., digital subsequences comprising m bits, in the digital sequence is termed n, wherein the value of n is at least one. As used herein, the term “at least one” encompasses 1, 2, 3, 4, 5, 6, 7, 8, 9, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 128, 256, 500, 512, 1000, 1024, 2048, 4096, 8192, 10⁴, 10⁵, 10⁶, 10⁷, 10⁸, 10⁹, 10¹⁰, 10¹¹, 10¹², 10¹³, 10¹⁴, 10¹⁵, 10¹⁶, 10¹⁷, 10¹⁸, 10¹⁹, 10²⁰, 10²¹ bytes, or more. Thus, in practice, the number of bits comprised in the digital sequence equals m (i.e., the number of bits per byte) multiplied by n (i.e., the number of bytes, or digital subsequences, comprised in the digital sequence).

Each bit has a defined position within the digital subsequence (or byte) comprising m bits, the first position being position 0, the last position being equal to m−1. Thus, the position of each bit in the digital sequence can be even or odd; wherein even positions comprise 0, 2, 4, 6, 8, 10, 12 and 14; and wherein odd positions comprise 1, 3, 5, 7, 9, 11, 13 and 15. In one embodiment, the digital subsequence is an octet; and even positions comprise 0, 2, 4 and 6, and odd positions comprise 1, 3, 5 and 7.

In one embodiment, the present invention comprises a step of converting a byte stored on an electronic device, into a byte stored on a nucleic acid molecule, wherein a byte stored on a nucleic acid molecule is herein referred to as a bioblock, and wherein a bioblock consists of m nucleotides. In one embodiment, the byte is an octet, i.e., m=8, and a bioblock is herein referred to as a biooctet.

In one embodiment, the bioblock comprises 2, 3 or 4 distinct nucleotides, wherein the distinct nucleotides are herein referred to as N1, N2, N3 and N4. In one embodiment, a biooctet comprises exactly 4 distinct nucleotides.

In one embodiment, both the value and position of each bit comprised in the byte is encoded in the corresponding bioblock, wherein:
bits having the value 0 and localized at even positions correspond to a first nucleotide N1,
bits having the value 1 and localized at even positions correspond to a second nucleotide N2,
bits having the value 0 and localized at odd positions correspond to a third nucleotide N3,
bits having the value 1 and localized at odd positions correspond to a fourth nucleotide N4, and
   wherein N1, N2, N3 and N4 are distinct nucleotides.

The method according to the invention comprises constructing at least one component, preferably more than one component, wherein each component comprises or consists of at least one bioblock (e.g., at least one biooctet), and wherein the total number of components is x. In one embodiment, the number of bioblocks (e.g., biooctet), per component is y, wherein the value of y is at least 1. In one embodiment, the value of x is n divided by

$$y ( x = \frac{n}{y} ) .$$

As used herein, the term “more than one” means 2, 3, 4, 5, 6, 7, 8, 9, 10, 20, 30, 40, 50, 100, 1000 or more. As used herein, the term “at least one” means 1, 2, 3, 4, 5, 6, 7, 8, 9, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 128, 256, 500, 512, 1000, 10⁴, 10⁵, 10⁶ or more.

In one embodiment, each component comprises the same number of bioblocks. In one embodiment, x=n, i.e., y=1.

In another embodiment, x and n are distinct, i.e., y≠1, meaning that each component comprises from 2 to n bioblocks (e.g., from 2 to n biooctet).

In certain embodiments, the value of x is not n divided by y.

In one embodiment, y does not have a fixed value, i.e., at least 2, 3, 4, 5 or more components comprise a distinct number of bioblocks. In certain embodiments, each component comprises a distinct number of bioblocks.

In certain embodiments, each component comprises the same number of bioblocks (y), except for one component that comprises from 1 to y−1 bioblocks, wherein the value of y is at least 2.

In one embodiment, the x components are assembled together in a fixed order, wherein the fixed order used for assembling the x components is identical to the order of the n digital subsequences within the digital sequence.

In one embodiment, the assembly of the x components is performed in one or more steps. In one embodiment, the assembly of the x components is performed in one step. In one embodiment, the assembly of the x components is performed in more than one step. In one embodiment, the assembly of the x components is performed sequentially, separately, simultaneously, or combinations thereof.

In one embodiment, the nucleotides are selected from the group consisting of natural nucleotides and non-natural nucleotides.

Natural nucleotides include adenine, guanine, cytosine, uracil and thymine.

Non-limitative examples of non-natural nucleotides include 2-Amino-ATP, 8-Aza-ATP, 2′-Fluoro-dATP, 2′-Fluoro-dCTP, 2′-Fluoro-dGTP, 2′-Fluoro-dUTP, 5-Iodo-CTP, 5-Iodo-UTP, N6-Methyl-ATP, 5-Methyl-CTP, 2′-O-Methyl-ATP, 2′-O-Methyl-CTP, 2′-O-Methyl-GTP, 2′-O-Methyl-UTP, Pseudo-UTP, ITP, 2′-O-Methyl-ITP, Puromycin-TP, Xanthosine-TP, 5-Methyl-UTP, 4-Thio-UTP, 2′-Amino-dCTP, 2′-Amino-dUTP, 2′-Azido-dCTP, 2′-Azido-dUTP, 06-Methyl-GTP, 2-Thio-UTP, Ara-CTP, Ara-UTP, 5,6-Dihydro-UTP, 2-Thio-CTP, 6-Aza-CTP, 6-Aza-UTP, N1-Methyl-GTP, 2′-O-Methyl-2-Amino-ATP, 2′-O-Methylpseudo-UTP, N1-Methyl-ATP, 2′-O-Methyl-5-methyl-UTP, 7-Deaza-GTP, 2′-Azido-dATP, 2′-Amino-dATP, Ara-ATP, 8-Azido-ATP, 5-Bromo-CTP, 5-Bromo-UTP, 2′-Fluoro-dTTP, 3′-O-Methyl-ATP, 3′-O-Methyl-CTP, 3′-O-Methyl-GTP, 3′-O-Methyl-UTP, 7-Deaza-ATP, 5-AA-UTP, 2′-Azido-dGTP, 2′-Amino-dGTP, 5-AA-CTP, 8-Oxo-GTP, Pseudoiso-CTP, N4-Methyl-CTP, N1-Methylpseudo-UTP, 5,6-Dihydro-5-Methyl-UTP, N6-Methyl-Amino-ATP, 5-Carboxy-CTP, 5-Formyl-CTP, 5-Hydroxymethyl-UTP, 5-Hydroxymethyl-CTP, Thieno-GTP, 5-Hydroxy-CTP, 5-Formyl-UTP, Thieno-UTP, 2-Amino-dATP, 5-Bromo-dCTP, 5-Bromo-dUTP, 7-Deaza-dATP, 7-Deaza-dGTP, dITP, 5-Propynyl-dCTP, 5-Propynyl-dUTP, 2′-dUTP, 5-Fluoro-dUTP, 5-Iodo-dCTP, 5-Iodo-dUTP, N6-Methyl-dATP, 5-Methyl-dCTP, 06-Methyl-dGTP, N2-Methyl-dGTP, 8-Oxo-dATP, 8-Oxo-dGTP, 2-Thio-dTTP, 2′-dPTP, 5-Hydroxy-dCTP, 4-Thio-dTTP, 2-Thio-dCTP, 6-Aza-dUTP, 6-Thio-dGTP, 8-Chloro-dATP, 5-AA-dCTP, 5-AA-dUTP, N4-Methyl-dCTP, 2′-deoxyzebularine-TP, 5-Hydroxymethyl-dUTP, 5-Hydroxymethyl-dCTP, 5-Propargylamino-dCTP, 5-Propargylamino-dUTP, 5-Carboxy-dCTP, 5-Formyl-dCTP, 5-Indolyl-AA-dUTP, 5-Carboxy-dUTP, 5-Formyl-dUTP, 3′-dATP, 3′-dGTP, 3′-dCTP, 5-Methyl-3′-dUTP, 3′-dUTP, ddATP, ddGTP, ddUTP, ddTTP, ddCTP, 3′-Azido-ddATP, 3′-Azido-ddGTP, 3′-Azido-ddTTP, 3′-Amino-ddATP, 3′-Amino-ddCTP, 3′-Amino-ddGTP, 3′-Amino-ddTTP, 3′-Azido-ddCTP, 3′-Azido-ddUTP, 5-Bromo-ddUTP, ddITP, (1-Thio)-dATP, (1-Thio)-dCTP, (1-Thio)-dGTP, (1-Thio)-dTTP, (1-Thio)-ATP, (1-Thio)-CTP, (1-Thio)-GTP, (1-Thio)-UTP, (1-Thio)-ddATP, (1-Thio)-ddCTP, (1-Thio)-ddGTP, (1-Thio)-ddTTP, (1-Thio)-3′-Azido-ddTTP, (1-Thio)-ddUTP, (1-Borano)-dATP, (1-Borano)-dCTP, (1-Borano)-dGTP, (1-Borano)-dTTP, Ganciclovir-TP, Cidofovir-DP, 3-methyl-6-amino-5-(1′-b-D-2′-deoxyribofuranosyl)-pyrimidin-2-one, 6-amino-9[(1′-b-D-2′-deoxyribofuranosyl)-4-hydroxy-5-(hydroxymethyl)-oxolan-2-yl]-1H-purin-2-one, 6-amino-3-(1′-b-D-2′-deoxyribofuranosyl)-5-nitro-1H-pyridin-2-one and 2-amino-8-(1′-b-D-2′-deoxyribofuranosyl)-imidazo-[1,2a]-1,3,5-triazin-[8H]-4-one.

In one embodiment, N1, N2, N3 and N4 are selected from the group comprising or consisting of adenine, guanine, cytosine, uracil, thymine and non-natural nucleotides. In one embodiment, N1, N2, N3 and N4 are selected from the group comprising or consisting of adenine, guanine, cytosine, uracil and thymine.

In one embodiment, N1, N2, N3 and N4 are selected from the group comprising or consisting of adenine, guanine, cytosine and thymine. In one embodiment, N1 is adenine, N2 is guanine, N3 is cytosine and N4 is thymine. In another embodiment, N1 is adenine, N2 is guanine, N3 is thymine and N4 is cytosine. In another embodiment, N1 is adenine, N2 is cytosine, N3 is thymine and N4 is guanine. In another embodiment, N1 is adenine, N2 is cytosine, N3 is guanine and N4 is thymine. In another embodiment, N1 is adenine, N2 is thymine, N3 is cytosine and N4 is guanine. In another embodiment, N1 is adenine, N2 is thymine, N3 is guanine and N4 is cytosine. In another embodiment, N1 is guanine, N2 is adenine, N3 is cytosine and N4 is thymine. In another embodiment, N1 is guanine, N2 is adenine, N3 is thymine and N4 is cytosine. In another embodiment, N1 is guanine, N2 is cytosine, N3 is adenine and N4 is thymine. In another embodiment, N1 is guanine, N2 is cytosine, N3 is thymine and N4 is adenine. In another embodiment, N1 is guanine, N2 is thymine, N3 is adenine and N4 is cytosine. In another embodiment, N1 is guanine, N2 is thymine, N3 is cytosine and N4 is adenine. In another embodiment, N1 is cytosine, N2 is adenine, N3 is guanine and N4 is thymine. In another embodiment, N1 is cytosine, N2 is adenine, N3 is thymine and N4 is guanine. In another embodiment, N1 is cytosine, N2 is guanine, N3 is adenine and N4 is thymine. In another embodiment, N1 is cytosine, N2 is guanine, N3 is thymine and N4 is adenine. In another embodiment, N1 is cytosine, N2 is thymine, N3 is adenine and N4 is guanine. In another embodiment, N1 is cytosine, N2 is thymine, N3 is guanine and N4 is adenine. In another embodiment, N1 is thymine, N2 is adenine, N3 is guanine and N4 is cytosine. In another embodiment, N1 is thymine, N2 is adenine, N3 is cytosine and N4 is guanine. In another embodiment, N1 is thymine, N2 is guanine, N3 is adenine and N4 is cytosine. In another embodiment, N1 is thymine, N2 is guanine, N3 is cytosine and N4 is adenine. In another embodiment, N1 is thymine, N2 is cytosine, N3 is adenine and N4 is guanine. In another embodiment, N1 is thymine, N2 is cytosine, N3 is guanine and N4 is adenine.

In one embodiment, N1, N2, N3 and N4 are selected from the group comprising or consisting of adenine, guanine, cytosine and uracil. In one embodiment, N1 is adenine, N2 is guanine, N3 is cytosine and N4 is uracil. In another embodiment, N1 is adenine, N2 is guanine, N3 is uracil and N4 is cytosine. In another embodiment, N1 is adenine, N2 is cytosine, N3 is uracil and N4 is guanine. In another embodiment, N1 is adenine, N2 is cytosine, N3 is guanine and N4 is uracil. In another embodiment, N1 is adenine, N2 is uracil, N3 is cytosine and N4 is guanine. In another embodiment, N1 is adenine, N2 is uracil, N3 is guanine and N4 is cytosine. In another embodiment, N1 is guanine, N2 is adenine, N3 is cytosine and N4 is uracil. In another embodiment, N1 is guanine, N2 is adenine, N3 is uracil and N4 is cytosine. In another embodiment, N1 is guanine, N2 is cytosine, N3 is adenine and N4 is uracil. In another embodiment, N1 is guanine, N2 is cytosine, N3 is uracil and N4 is adenine. In another embodiment, N1 is guanine, N2 is uracil, N3 is adenine and N4 is cytosine. In another embodiment, N1 is guanine, N2 is uracil, N3 is cytosine and N4 is adenine. In another embodiment, N1 is cytosine, N2 is adenine, N3 is guanine and N4 is uracil. In another embodiment, N1 is cytosine, N2 is adenine, N3 is uracil and N4 is guanine. In another embodiment, N1 is cytosine, N2 is guanine, N3 is adenine and N4 is uracil. In another embodiment, N1 is cytosine, N2 is guanine, N3 is uracil and N4 is adenine. In another embodiment, N1 is cytosine, N2 is uracil, N3 is adenine and N4 is guanine. In another embodiment, N1 is cytosine, N2 is uracil, N3 is guanine and N4 is adenine. In another embodiment, N1 is uracil, N2 is adenine, N3 is guanine and N4 is cytosine. In another embodiment, N1 is uracil, N2 is adenine, N3 is cytosine and N4 is guanine. In another embodiment, N1 is uracil, N2 is guanine, N3 is adenine and N4 is cytosine. In another embodiment, N1 is uracil, N2 is guanine, N3 is cytosine and N4 is adenine. In another embodiment, N1 is uracil, N2 is cytosine, N3 is adenine and N4 is guanine. In another embodiment, N1 is uracil, N2 is cytosine, N3 is guanine and N4 is adenine.

In one embodiment, N1, N2, N3 and N4 are selected from the group comprising or consisting of non-natural nucleotides.

In one embodiment, the x components are nucleic acid molecules selected from the group comprising or consisting of double-stranded DNA molecules, single-stranded DNA molecules, double-stranded RNA molecules, single-stranded RNA molecules, and nucleic acid molecules comprising at least one non-natural nucleotide.

In one embodiment, the x components are x DNA molecules, preferably x double-stranded DNA molecules.

In one embodiment, the x components are double stranded DNA molecules. In one embodiment, the x components are single stranded DNA molecules.

In another embodiment, the x components are double stranded RNA molecules or single stranded RNA molecules. In another embodiment, the x components are nucleic acid molecules comprising at least one non-natural nucleotide.

In one embodiment, the construction of a plurality of x components, each comprising at least one bioblock, comprises the steps of:
selectively capturing x data storage nucleic acid molecules from at least one library of data storage nucleic acid molecules, wherein each data storage nucleic acid molecule comprises at least one bioblock surrounded by regions comprising cleavage sites,
cleaving each of the x data storage nucleic acid molecules, thereby releasing the at least one bioblock.

Within the scope of the present invention, the “data storage nucleic acid molecule” is a molecule, typically a plasmid, comprising at least one bioblock (e.g., at least one biooctet), or component according to the invention, wherein each bioblock (e.g., biooctet) or component is flanked by regions comprising cleavage sites. In one embodiment, the data storage nucleic acid molecule comprises or consists of nucleotides selected from the group comprising or consisting of natural and non-natural nucleotides.

Within the scope of the present invention, the term “library of data storage nucleic acid molecules” refers to a definite plurality of data storage nucleic acid molecules as defined herein, wherein each data storage nucleic acid molecule of the library comprises distinct bioblocks (e.g., biooctets) or components.

As used herein, the term “cleavage site” refers to a nucleotide sequence targeted by an enzyme selected from the group comprising or consisting of restriction enzymes (also referred to as restriction endonucleases), endonucleases, exonucleases, deoxyribonuclease, ribonuclease, nickases, transposases and integrases. In a preferred embodiment, the enzyme is a site-directed enzyme, i.e., an enzyme that recognizes a specific nucleic acid sequence.

In one embodiment, the cleavage sites are targeted by restriction enzymes. In one embodiment, the cleavage sites are restriction sites. As used herein, the term “restriction site” refers to a nucleotide sequence targeted by a specific restriction enzyme. Non-limitative examples of restriction enzymes include EcoRI, BamHI, HindIII, KpnI, NotI, PstI, SmaI and XhoI. Restriction enzymes and corresponding restriction sites are well known in the art.

In another embodiment, the cleavage sites are targeted by enzymes selected from the group comprising or consisting of endonucleases, exonucleases, deoxyribonucleases, ribonucleases, nickases, integrases and transposases.

In one embodiment, the region comprising cleavage sites comprises a first nucleotide sequence that is recognized by the enzyme, typically a restriction enzyme, and a second nucleotide sequence that is digested, or cleaved, by the enzyme. In one embodiment, the first nucleotide sequence and the second nucleotide sequence are distinct. In certain embodiments, the first nucleotide sequence and the second nucleotide sequence are separated by at least one nucleotide. In one embodiment, the digestion of the cleavage site separates the first nucleotide sequence from the second nucleotide sequence.

In one embodiment, the digestion of the cleavage site produces protruding ends or blunt ends, preferably protruding ends. Within the scope of the present invention, these protruding ends are hereby referred to as “fusion sites”. In one embodiment, the protruding end is 3′ protruding or 5′ protruding. In one embodiment, the nucleotide sequences of the 3′ protruding end and the 5′ protruding end are complementary.

In one embodiment, the construction of a plurality of x components, each comprising at least one bioblock (e.g., biooctet), comprises the steps of:
selectively capturing n data storage nucleic acid molecules from at least two libraries of data storage nucleic acid molecules, wherein each data storage nucleic acid molecule of each library comprises one bioblock (e.g., biooctet) surrounded by regions comprising cleavage sites, and wherein each library comprises all possible bioblocks of m nucleotides (e.g., all possible biooctets of 8 nucleotides),
cleaving each of the n data storage nucleic acid molecules, thereby releasing the n bioblocks (e.g., biooctets).

In one embodiment, the regions comprising cleavage sites comprises from 2 to 25 nucleotides.

As used herein, the expression “from 2 to 25 nucleotides” comprises 2, 3, 4, 5, 6, 7, 8, 9, 10, 11, 12, 13, 14, 15, 16, 17, 18, 19, 20, 21, 22, 23, 24 and 25 nucleotides.

In one embodiment, the regions comprising cleavage sites comprises from 2 to 20 nucleotides. In one embodiment, the regions comprising cleavage sites comprises from 2 to 15 nucleotides. In one embodiment, the regions comprising cleavage sites comprises from 2 to 10 nucleotides.

In one embodiment, the cleavage sites are localized both upstream and downstream of the bioblock (e.g., biooctet) or component.

As used herein, the term “upstream” refers to a position:
Adjacent in 5′ of the most 5′ end of the sequence of the bioblock (e.g., biooctet) or component, if the data storage nucleic acid molecule is a single stranded nucleic acid molecule, wherein adjacent means either contiguous or separated by a spacer (e.g., 1, 2, 3, 4, 5, 6, 7, 8, 9, 10 or more nucleotides); or
Adjacent in 5′ of the most 5′ end of the sequence of the bioblock (e.g., biooctet) or component on the positive strand, and in 3′ of the most 3′ end of the sequence of the bioblock (e.g., biooctet) or component on the negative strand, if the data storage nucleic acid molecule is a double stranded nucleic acid molecule, wherein adjacent means either contiguous or separated by a spacer (e.g., 1, 2, 3, 4, 5, 6, 7, 8, 9, 10 or more nucleotides.

As used herein, the term “downstream” refers to a position:
Adjacent in 3′ of the most 3′ end of the sequence of the bioblock (e.g., biooctet) or component, if the data storage nucleic acid molecule is a single stranded nucleic acid molecule, wherein adjacent means either contiguous or separated by a spacer (e.g., 1, 2, 3, 4, 5, 6, 7, 8, 9, 10 or more nucleotides; or
Adjacent in 3′ of the most 3′ end of the sequence of the bioblock (e.g., biooctet) or component on the positive strand, and in 5′ of the most 5′ end of the sequence of the bioblock (e.g., biooctet) or component on the negative strand, if the data storage nucleic acid molecule is a double stranded nucleic acid molecule, wherein adjacent means either contiguous or separated by a spacer (e.g., 1, 2, 3, 4, 5, 6, 7, 8, 9, 10 or more nucleotides.

In one embodiment, the data storage nucleic acid molecule comprises a number of upstream regions comprising cleavage sites that is identical to the number of downstream regions comprising cleavage sites. In one embodiment, the data storage nucleic acid molecule comprises at least 1 upstream region comprising a cleavage site and at least 1 downstream region comprising a cleavage site. In one embodiment, the data storage nucleic acid molecule comprises 1 upstream region comprising a cleavage site and 1 downstream region comprising a cleavage site. In one embodiment, the data storage nucleic acid molecule comprises 2 upstream regions comprising cleavage sites and 2 downstream regions comprising cleavage sites.

In one embodiment, the data storage nucleic acid molecule may comprise at least two distinct cleavage sites, wherein distinct cleavage sites have distinct nucleic acid sequence, preferably wherein distinct cleavage sites are digested by distinct enzymes.

In another embodiment, the upstream cleavage site and the downstream cleavage are similar and cleaved by distinct enzymes. In another embodiment, the upstream cleavage site and the downstream cleavage are similar and cleaved by the same enzyme.

In a preferred embodiment, the upstream cleavage site and the downstream cleavage site are distinct and cleaved by the same enzyme.

In one embodiment, the data storage nucleic acid molecule further comprises 2 additional cleavage sites, wherein the first one is localized upstream of the bioblocks (e.g., biooctets) or components and the second one is localized downstream of the bioblocks (e.g., biooctets) or components.

In one embodiment, the 2 additional cleavage sites are distinct and cleaved by the same enzyme. In another embodiment, the 2 additional cleavage sites are distinct and cleaved by distinct enzymes. In another embodiment, the 2 additional cleavage sites are similar and cleaved by the same enzyme. In another embodiment, the 2 additional cleavage sites are similar and cleaved by distinct enzymes.

In one embodiment, the 2 additional cleavage sites are distinct from the other cleavage sites comprised on the data storage nucleic acid molecule and are cleaved by enzymes distinct from those cleaving the cleavage sites comprised on the data storage nucleic acid molecule. In another embodiments, the 2 additional cleavage sites are similar from the other cleavage sites comprised on the data storage nucleic acid molecule and are cleaved by enzymes similar to those cleaving the cleavage sites comprised on the data storage nucleic acid molecule.

In one embodiment, the bioblocks (e.g., biooctet) or components are considered released when at least one upstream cleavage site and at least one downstream cleavage site are cleaved (i.e., digested or cut).

In one embodiment, a released bioblock (e.g., biooctet) comprises (i) one bioblock (e.g., biooctet), (ii) part of the closest upstream cleavage site, i.e., the upstream fusion site, and (iii) part of the closest downstream cleavage site, i.e., the downstream fusion site. In one embodiment, the part of the closest upstream cleavage site, i.e., the upstream fusion site, is a protruding end (e.g., 3′ protruding end). In one embodiment, the part of the closest downstream cleavage site, i.e., the downstream fusion site, is a protruding end (e.g., 5′ protruding end).

In one embodiment, a released component comprises (i) at least one bioblock (e.g., biooctet), (ii) part of the closest upstream cleavage site, i.e., the upstream fusion site, and (iii) part of the closest downstream cleavage site, i.e., the downstream fusion site. In a preferred embodiment, a released component comprises (i) y bioblocks (e.g., biooctets), (ii) part of the closest upstream cleavage site, i.e., the upstream fusion site, and (iii) part of the closest downstream cleavage site, i.e., the downstream fusion site.

In one embodiment, assembling together a plurality of x components involves releasing bioblocks (e.g., biooctets) or components. In one embodiment, releasing bioblocks (e.g., biooctets) or components involves using either one enzyme or two distinct enzymes.

In one embodiment, each of the region surrounding each bioblock (e.g., biooctet) comprises a site for a restriction enzyme, and step (d) of the method of the invention comprises a step of digesting each of the x data storage nucleic acid molecules with one or two restriction enzymes.

In another embodiment, each of the region surrounding each bioblock (e.g., biooctet) comprises a site for a restriction enzyme, and step (d) of the method of the invention comprises a step of digesting each of the x data storage nucleic acid molecules with two restriction enzymes.

In one embodiment, digestion of the upstream restriction site produces a 3′ protruding end or a 5′ protruding end, digestion of the downstream restriction site produces a 3′ protruding end or a 5′ protruding end. In one embodiment, the nucleotide sequences of the 3′ protruding end and the 5′ protruding end are complementary.

In one embodiment, the restriction site comprises a first nucleotide sequence that is recognized by the restriction enzyme, and a second nucleotide sequence that is digested, or cleaved, by the enzyme. In one embodiment, the first nucleotide sequence and the second nucleotide sequence are distinct. In certain embodiments, the first nucleotide sequence and the second nucleotide sequence are separated by at least one nucleotide. In one embodiment, the digestion of the restriction site separates the first nucleotide sequence from the second nucleotide sequence.

In one embodiment, the restriction enzymes are selected from the group comprising or consisting of type I, type II, type III, type IV or type V restriction enzymes, or combinations thereof. In one embodiment, the restriction enzyme is a type II restriction enzyme. In one embodiment, the type II restriction enzymes are selected from the group comprising or consisting of type II S, type II G, type II B, type II T and/or type II C restriction enzymes, or combination thereof, preferably type II S and/or type II G, more preferably type II S. Non-limitative examples of type II S restriction enzymes include BsaI, BbsI, BsmBI, FokI, Alw26I, BbvI, BsrI, Earl, HphI, MboII, SfaNI and Tth111I. In one embodiment, the restriction enzymes are BsaI and/or BbsI and/or BsmBI.

In one embodiment, the restriction enzymes are modified. In one embodiment, the restriction enzymes comprise at least one mutation in their amino acid sequence compared to the unmodified (or wild type) amino acid sequence. In one embodiment, the restriction enzymes are post-translationally modified.

In certain embodiments, the enzyme recognition sites consist of a nucleotide sequence selected from GGTCTC and CGTCTC.

In one embodiment, the cleavage sites comprise a nucleotide sequence selected from the group comprising or consisting of GTAG, TGAC, TCAG, AATA, TCAA, CTTC, AGTA, ACTG, CACA, CCAG, CAAA, GACC, ACTC, CCAC, GAAC, GCAC, CGGC, CGTA, GTAA, CAAC, GCTA, CCGA, ACGA, AGAA, TAAA, AGCG, ACCT, AACA, GGCA, ACGC, AATC, CGAG, TCCA, CCTA, CTAA, GGGA, AAGG, AAAC, CTAC, and GAGA. In one embodiment, these sequences are protruding ends.

In one embodiment, the fusion sites comprise a nucleotide sequence selected from the group comprising or consisting of GTAG, TGAC, TCAG, AATA, TCAA, CTTC, AGTA, ACTG, CACA, CCAG, CAAA, GACC, ACTC, CCAC, GAAC, GCAC, CGGC, CGTA, GTAA, CAAC, GCTA, CCGA, ACGA, AGAA, TAAA, AGCG, ACCT, AACA, GGCA, ACGC, AATC, CGAG, TCCA, CCTA, CTAA, GGGA, AAGG, AAAC, CTAC, and GAGA.

In one embodiment, the cleavage sites comprise a nucleotide sequence selected from the group comprising or consisting of GTAG, TGAC, TCAG. In one embodiment, the cleavage sites comprise a nucleotide sequence selected from the group comprising or consisting of AATA, TCAA, CTTC, AGTA, ACTG, CACA, CCAG, CAAA, GACC, ACTC, CCAC, GAAC, GCAC, CGGC, CGTA, GTAA, CAAC, GCTA, CCGA, ACGA, AGAA, TAAA, AGCG, ACCT, AACA, GGCA, ACGC, AATC, CGAG, TCCA, CCTA, CTAA and GGGA. In one embodiment, the cleavage sites comprise a nucleotide sequence selected from the group comprising or consisting of AATA, AAGG, AAAC, TAAA, ACGA, ACTG, AGCG, GCTA, GGCA, ACCT, CGTA, AACA, CTAC, GAGA, CCAG, AGAA and GCAC.

In one embodiment, the fusion sites comprise a nucleotide sequence selected from the group comprising or consisting of GTAG, TGAC, TCAG. In one embodiment, the fusion sites comprise a nucleotide sequence selected from the group comprising or consisting of AATA, TCAA, CTTC, AGTA, ACTG, CACA, CCAG, CAAA, GACC, ACTC, CCAC, GAAC, GCAC, CGGC, CGTA, GTAA, CAAC, GCTA, CCGA, ACGA, AGAA, TAAA, AGCG, ACCT, AACA, GGCA, ACGC, AATC, CGAG, TCCA, CCTA, CTAA and GGGA. In one embodiment, the fusion sites comprise a nucleotide sequence selected from the group comprising or consisting of AATA, AAGG, AAAC, TAAA, ACGA, ACTG, AGCG, GCTA, GGCA, ACCT, CGTA, AACA, CTAC, GAGA, CCAG, AGAA and GCAC.

In one embodiment, step (e) comprises one or several assembling steps using overlap-extension polymerase chain reaction (PCR), polymerase cycling assembly, sticky end ligation, biobricks assembly, golden gate assembly, Gibson assembly, recombinase assembly, ligase cycling reaction, template directed ligation, in vivo assembly or any other DNA assembly protocol.

In one embodiment, step (e) comprises one or several assembling steps using overlap PCR. In one embodiment, step (e) comprises one or several assembling steps using polymerase cycling assembly. In one embodiment, step (e) comprises one or several assembling steps using sticky end ligation. In one embodiment, step (e) comprises one or several assembling steps using biobricks assembly. In one embodiment, step (e) comprises one or several assembling steps using golden gate assembly. In one embodiment, step (e) comprises one or several assembling steps using Gibson assembly. In one embodiment, step (e) comprises one or several assembling steps using recombinase assembly. In one embodiment, step (e) comprises one or several assembling steps using ligase cycling reaction. In one embodiment, step (e) comprises one or several assembling steps using template directed ligation. In one embodiment, step (e) comprises one or several assembling steps using in vivo assembly.

In one embodiment, step (e) comprises using a ligase.

In a preferred embodiment, the cleavage of the regions comprising cleavage sites produces protruding ends, also referred to as fusion sites. In a preferred embodiment, the closest fusion site on one end (e.g., 3′ end) of the first bioblock (e.g., biooctet) or component, and the closest fusion site on the other end (e.g., 5′ end) of the second bioblock (e.g., biooctet) or component are complementary.

In one embodiment, the assembly of components comprising at least one bioblock (e.g., biooctets) necessitates or is facilitated by the complementarity between:
the closest fusion site on one end (e.g., 3′ end) of a first bioblock (e.g., biooctet), and
the closest fusion site on the other end (e.g., 5′ end) of a second bioblock (e.g., biooctet).

In one embodiment, the nucleotide sequence recognized by the enzyme is not comprised on the nucleotide sequence digested by the enzyme. In one embodiment, upon digestion of the cleavage site, the nucleotide sequence recognized by the enzyme is lost, i.e., it is separated from the cleaved sequence. In one embodiment, the cleavage sites between 2 bioblocks (e.g., between 2 biooctets) or 2 components do not comprise the nucleotide sequence recognized by the enzyme.

In one embodiment, an assembled component comprising y bioblocks (e.g., biooctets) comprises or consists of:
y bioblocks (e.g., biooctets) in a fixed order,
y+1 fusion sites flanking the bioblocks.

In one embodiment, an assembled component comprising y bioblocks (e.g., biooctets) comprises or consists of:
y bioblocks (e.g., biooctets) in a fixed order,
y+1 fusion sites flanking the bioblocks, and
2 regions comprising cleavage sites, wherein the regions comprising the cleavage sites are localized at the furthest 5′ end and the furthest 3′ end of the component.

The present invention further relates to a data storage nucleic acid molecule comprising at least one bioblock, a bioblock consisting of a nucleic acid sequence consisting of m nucleotides assigned to positions 0 to m−1, wherein
a bioblock is formed of at least 2 and at most 4 (i.e., 2, 3 or 4) distinct nucleotides
nucleotides at even positions may be selected from a first and a second nucleotide, and nucleotides at odd positions may be selected from a third and a fourth nucleotide, said first, second, third and fourth nucleotides being distinct.

In one embodiment, the first, second, third and fourth nucleotides are referred to as N1, N2, N3 and N4, respectively.

In one embodiment, N1, N2, N3 and N4 are selected from the group comprising or consisting of adenine, guanine, cytosine, uracil, thymine and non-natural nucleotides, wherein N1, N2, N3 and N4 are distinct nucleotides. In one embodiment, N1, N2, N3 and N4 are selected from the group comprising or consisting of adenine, guanine, cytosine, uracil and thymine, wherein N1, N2, N3 and N4 are distinct nucleotides.

In one embodiment, N1, N2, N3 and N4 are selected from the group comprising or consisting of adenine, guanine, cytosine and thymine, wherein N1, N2, N3 and N4 are distinct nucleotides. In one embodiment, N1 is adenine, N2 is guanine, N3 is cytosine and N4 is thymine. In another embodiment, N1 is adenine, N2 is guanine, N3 is thymine and N4 is cytosine. In another embodiment, N1 is adenine, N2 is cytosine, N3 is thymine and N4 is guanine. In another embodiment, N1 is adenine, N2 is cytosine, N3 is guanine and N4 is thymine. In another embodiment, N1 is adenine, N2 is thymine, N3 is cytosine and N4 is guanine. In another embodiment, N1 is adenine, N2 is thymine, N3 is guanine and N4 is cytosine. In another embodiment, N1 is guanine, N2 is adenine, N3 is cytosine and N4 is thymine. In another embodiment, N1 is guanine, N2 is adenine, N3 is thymine and N4 is cytosine. In another embodiment, N1 is guanine, N2 is cytosine, N3 is adenine and N4 is thymine. In another embodiment, N1 is guanine, N2 is cytosine, N3 is thymine and N4 is adenine. In another embodiment, N1 is guanine, N2 is thymine, N3 is adenine and N4 is cytosine. In another embodiment, N1 is guanine, N2 is thymine, N3 is cytosine and N4 is adenine. In another embodiment, N1 is cytosine, N2 is adenine, N3 is guanine and N4 is thymine. In another embodiment, N1 is cytosine, N2 is adenine, N3 is thymine and N4 is guanine. In another embodiment, N1 is cytosine, N2 is guanine, N3 is adenine and N4 is thymine. In another embodiment, N1 is cytosine, N2 is guanine, N3 is thymine and N4 is adenine. In another embodiment, N1 is cytosine, N2 is thymine, N3 is adenine and N4 is guanine. In another embodiment, N1 is cytosine, N2 is thymine, N3 is guanine and N4 is adenine. In another embodiment, N1 is thymine, N2 is adenine, N3 is guanine and N4 is cytosine. In another embodiment, N1 is thymine, N2 is adenine, N3 is cytosine and N4 is guanine. In another embodiment, N1 is thymine, N2 is guanine, N3 is adenine and N4 is cytosine. In another embodiment, N1 is thymine, N2 is guanine, N3 is cytosine and N4 is adenine. In another embodiment, N1 is thymine, N2 is cytosine, N3 is adenine and N4 is guanine. In another embodiment, N1 is thymine, N2 is cytosine, N3 is guanine and N4 is adenine.

In one embodiment, N1, N2, N3 and N4 are selected from the group comprising or consisting of adenine, guanine, cytosine and uracil, wherein N1, N2, N3 and N4 are distinct nucleotides. In one embodiment, N1 is adenine, N2 is guanine, N3 is cytosine and N4 is uracil. In another embodiment, N1 is adenine, N2 is guanine, N3 is uracil and N4 is cytosine. In another embodiment, N1 is adenine, N2 is cytosine, N3 is uracil and N4 is guanine. In another embodiment, N1 is adenine, N2 is cytosine, N3 is guanine and N4 is uracil. In another embodiment, N1 is adenine, N2 is uracil, N3 is cytosine and N4 is guanine. In another embodiment, N1 is adenine, N2 is uracil, N3 is guanine and N4 is cytosine. In another embodiment, N1 is guanine, N2 is adenine, N3 is cytosine and N4 is uracil. In another embodiment, N1 is guanine, N2 is adenine, N3 is uracil and N4 is cytosine. In another embodiment, N1 is guanine, N2 is cytosine, N3 is adenine and N4 is uracil. In another embodiment, N1 is guanine, N2 is cytosine, N3 is uracil and N4 is adenine. In another embodiment, N1 is guanine, N2 is uracil, N3 is adenine and N4 is cytosine. In another embodiment, N1 is guanine, N2 is uracil, N3 is cytosine and N4 is adenine. In another embodiment, N1 is cytosine, N2 is adenine, N3 is guanine and N4 is uracil. In another embodiment, N1 is cytosine, N2 is adenine, N3 is uracil and N4 is guanine. In another embodiment, N1 is cytosine, N2 is guanine, N3 is adenine and N4 is uracil. In another embodiment, N1 is cytosine, N2 is guanine, N3 is uracil and N4 is adenine. In another embodiment, N1 is cytosine, N2 is uracil, N3 is adenine and N4 is guanine. In another embodiment, N1 is cytosine, N2 is uracil, N3 is guanine and N4 is adenine. In another embodiment, N1 is uracil, N2 is adenine, N3 is guanine and N4 is cytosine. In another embodiment, N1 is uracil, N2 is adenine, N3 is cytosine and N4 is guanine. In another embodiment, N1 is uracil, N2 is guanine, N3 is adenine and N4 is cytosine. In another embodiment, N1 is uracil, N2 is guanine, N3 is cytosine and N4 is adenine. In another embodiment, N1 is uracil, N2 is cytosine, N3 is adenine and N4 is guanine. In another embodiment, N1 is uracil, N2 is cytosine, N3 is guanine and N4 is adenine.

In some embodiments, N1, N2, N3 and N4 are non-natural nucleotides as described hereinabove, wherein N1, N2, N3 and N4 are distinct nucleotides.

In one embodiment, the data storage nucleic acid molecule is a double-stranded molecule, preferably a DNA molecule.

In one embodiment, the double stranded nucleic acid molecule is circular or linear, preferably circular. In one embodiment, the data storage nucleic acid molecule is a linear sequence that has been circularized. Method to circularize a DNA sequence are known in the art.

In one embodiment, the data storage nucleic acid molecule is a plasmid, a cosmid, a fosmid, a prokaryotic chromosome (e.g., bacterial artificial chromosome) or a eukaryotic chromosome (e.g., yeast artificial chromosome or human artificial chromosome).

In a preferred embodiment, the data storage nucleic acid molecule is a plasmid. In another embodiment, the data storage nucleic acid molecule is a cosmid. In another embodiment, the data storage nucleic acid molecule is a fosmid. In another embodiment, the data storage nucleic acid molecule is a prokaryotic chromosome. In another embodiment, the data storage nucleic acid molecule is a eukaryotic chromosome.

In one embodiment, in the data storage nucleic acid molecule, each of the bioblocks (e.g., biooctets) or component is surrounded by regions comprising at least one cleavage site. In one embodiment, in the data storage nucleic acid molecule, each of the bioblocks (e.g., biooctets) or component is surrounded by regions comprising one cleavage site. In another embodiment, in the data storage nucleic acid molecule, each of the bioblocks (e.g., biooctets) or component is surrounded by regions comprising two cleavage sites, wherein the cleavage sites within the same region are distinct.

In one embodiment, the digestion of the regions comprising cleavage site by the restriction enzymes produces protruding end or blunt ends, preferably protruding ends (i.e., fusion sites). In one embodiment, protruding ends are 3′ protruding or 5′ protruding.

In one embodiment, a data storage nucleic acid molecule comprises at least one component, and each of the component is surrounded by regions comprising one or more cleavage sites.

In one embodiment, the data storage nucleic acid molecule is replicative.

As used herein, the “replicative” property of the data storage nucleic acid molecule according to the invention refers to its ability to be duplicated one or more time(s) in vivo in a living organism, in particular by a polymerase, more particularly by a DNA polymerase.

In one embodiment, the assessment of the replicative property of a nucleic acid molecule may be performed according to any standard method from the state of the art, or a method derived therefrom. Illustratively, the replicative property may be assessed by the increase of the number of copies of said nucleic acid molecules in/by a living organism and/or the ability of the living organism to transfer the nucleic acid to its progeny.

In one embodiment, the living organism is a microorganism, in particular a bacterium, a microalga, an archaeon, a fungus, a phage, a virus or a yeast. In one embodiment, the living organism is a prokaryote. Non-limitative examples of prokaryotes according to the invention include bacteria, such as actinobacteria, chlamydiales, cyanobacteria, firmicutes, proteobacteria, spirochetes, thermotogales; and archaea, such as euarchaeota, crenarchaeota. In one embodiment, the living organism is a bacterium, preferably Escherichia coli, more preferably Escherichia coli strain DH5a.

In certain embodiments, the living organism is a eukaryote. Non-limitative examples of eukaryotes according to the invention include protozoa, algae, plants, fungi, animals and their respective cells thereof.

In order to be replicated, the data storage nucleic acid molecule according to the invention possesses at least one origin of replication, namely one or more sequence(s) of nucleotides recognized by a replication initiation machinery. Illustratively, archaeon and bacterial origins of replication include oriC. In practice, most bacteria may have a unique origin of replication; an archaeon may have one or more origin(s) of replication; a eukaryote may have multiple origins of replication, in particular in the form of centromeres. Within the scope of the instant invention, the term “multiple origins of replication” refers to at least 2, 3, 4, 5, 10, 15, 20, 25, 50, 75, 100, 150, 200 origins of replication per nucleic acid molecule.

In one embodiment, the data storage nucleic acid molecule comprises or consists of (i) at least one component as described hereinabove, and (ii) at least one origin of replication.

In one embodiment, the data storage nucleic acid molecule does not comprise a promoter region. In one embodiment, the data storage nucleic acid molecule does not comprise a biological coding sequence.

In one embodiment, the data storage nucleic acid molecule is non-coding.

In one embodiment, the size of the data storage nucleic acid molecule is comprised between 100 base pairs (bp) and 1·10⁶ bp. As used herein, the expression “between 100 base pairs (bp) and 10⁶ bp” comprises 100, 200, 300, 400, 500, 600, 700, 800, 900, 1000, 5000, 10⁴, 10⁵, and 10⁶ bp.

In some embodiments, the data storage nucleic acid molecule further comprises one or more regions carrying metadata, i.e., information that do not encode digital information. Typically, these regions are termed “metadata bioblocks” (e.g., metadata biooctet).

In some embodiments, the metadata region comprises or consists of at least one barcoding region. As used herein, the term “barcoding region” refers to a bioblock (e.g., biooctet) added at the beginning of a component, or group of components. Typically, the barcode encodes a number (e.g., 0, 1, 2, 3, 4 and the like) using the same encoding system as the bioblocks, and the numbering system allows to label the components, or group of components, in a definite order.

In some embodiments, the metadata region comprises or consists of a “end of file” signal. As used herein, the term “end of file signal” refers to a special bioblock (e.g., biooctet) with a predefined sequence that is not shared with any other bioblock, that is localized at the end of the sequence. Typically, the “end of file” signal indicates the end of the region encoding digital data of the file.

In some embodiments, the metadata region comprises or consists of at least one barcoding region and one “end of file signal”, as described hereinabove.

The present invention further relates to a library comprising a plurality of data storage nucleic acid molecules according to the invention, wherein each of the data storage nucleic acid molecule of the library contains one bioblock (e.g., biooctet), wherein each data storage nucleic acid molecule of the library comprises the same surrounding regions comprising cleavage sites and wherein the library contains all possible bioblocks of m nucleotides.

In one embodiment, each data storage molecule of the library comprises exactly one bioblock (e.g., biooctet). In one embodiment, the total number of data storage nucleic acid molecules in the library is equal to 2^{m}. In one embodiment, m=8; thus, the size of the library is 256 data storage nucleic acid molecules.

In one embodiment, each data storage molecule of the library comprises a distinct bioblock (e.g., biooctet). In practice, a library comprises 2^{m} distinct bioblocks (e.g., biooctets).

In one embodiment, two distinct libraries comprise distinct bioblocks (e.g., biooctets). In another embodiment, two distinct libraries may comprise at least one common (i.e., identical) bioblock (e.g., biooctet). In certain embodiments, two distinct libraries comprise more than 2^{m} distinct bioblocks (e.g., biooctets).

In another embodiment, each data storage molecule comprises components according to the invention, wherein each component comprises more than one bioblock (e.g., biooctet). In one embodiment, each data storage molecule of the library comprises at least 1 component. In one embodiment, each data storage molecule of the library comprises a distinct component. In one embodiment, two distinct libraries comprise distinct components. In another embodiment, two distinct libraries may comprise at least one common (i.e., identical) component.

In one embodiment, each data storage molecule of the library comprises from 1 to 32 components. As used herein, the expression from 1 to 32 encompasses 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31 and 32. In one embodiment, each data storage molecule of the library comprises from 2 to 32 components. In one embodiment, each data storage molecule of the library comprises from 4 to 32 components. In one embodiment, each data storage molecule of the library comprises from 8 to 32 components. In one embodiment, each data storage molecule of the library comprises from 16 to 32 components. In one embodiment, each data storage molecule of the library comprises from 1 to 16 components. In one embodiment, each data storage molecule of the library comprises from 1 to 8 components. In one embodiment, each data storage molecule of the library comprises from 1 to 4 components. In one embodiment, each data storage molecule of the library comprises from 1 to 2 components.

In another embodiment, each data storage molecule of the library comprises more than 32 components.

In some embodiments, libraries comprising data storage molecule comprising at least one component are assembled using the bioblocks (e.g., biooctets) released from at least one library comprising data storage molecule comprising exactly one bioblock (e.g., biooctet), using the method as disclosed in the present invention. In practice, a nucleic acid molecule comprising exactly one couple of cleavage sites identical to the cleavage sites flanking the bioblocks (e.g., biooctets), herein referred to as acceptor molecule, is digested using at least one enzyme, preferably one enzyme, and is assembled with at least one bioblock (e.g., biooctet) using the method as described hereinabove.

In some embodiments, libraries comprising data storage molecules comprising more than one component are assembled using the components released from at least one library comprising data storage molecule comprising exactly one component, using the method as disclosed in the present invention.

In one embodiment, the regions comprising cleavage sites comprised on each data storage molecule of the library are identical.

In one embodiment, data storage molecules of distinct libraries comprise distinct regions comprising cleavage sites.

In one embodiment, data storage nucleic acid molecules of distinct libraries comprise identical regions comprising cleavage sites, wherein the bioblocks (e.g., biooctets) or components comprised in the data storage molecule of the first library are not used to assemble components comprised in the data storage molecule of the second library, and wherein the bioblocks (e.g., biooctets) or components comprised in the data storage molecule of the second library are not used to assemble components comprised in the data storage molecule of the first library.

In one embodiment, components may be assembled using bioblocks (e.g., biooctets) or components from more than one library.

In one embodiment, the data storage nucleic acid molecules comprised in the library are identified and labelled according to:
the nucleic acid sequence of the bioblocks (e.g., biooctets) and/or components they comprise, and/or
the nucleic acid sequence, or region, comprising cleavage sites surrounding the bioblocks (e.g., biooctets) and/or components, and/or
the encoding system used to convert digital subsequences comprising m bits (i.e., value and position of the bits) into bioblocks, according to the method of the invention. In one embodiment, the encoding system is displayed in the form “(N1, N2, N3, N4)”, “(N1, N2, N3)” or “(N1, N2)”.

In one embodiment, the labelling information is digital and/or physical. In one embodiment, the labelling information is stored in at least one database.

In one embodiment, data storage nucleic acid molecules comprised in the library are labelled using a code or an identifier that does not provide any information regarding the content of the data storage nucleic acid molecules. In one embodiment the information regarding the sequence of the data storage nucleic acid molecules and the encoding system are retrieved by searching for the corresponding code or identifier within the at least one database.

In a preferred embodiment, the data storage nucleic acid molecules comprised in the library are stored separately.

In one embodiment, the data storage nucleic acid molecules of a library are stored at a temperature suitable for preventing nucleic acid degradation. In one embodiment, the data storage nucleic acid molecules comprised in the library are stored at a temperature comprised from 4° C. to −200° C. As used herein, the expression “from 4° C. to −200° C.” encompasses 4, 3, 2, 1, 0, −1, −2, −3, −4, −5, −6, −7, −8, −9, −10, −11, −12, −13, −14, −15, −16, −17, −18, −19, −20, −30, −40, −50, −60, −70, −80, −90, −100, −120, −140, −160, −180, −200° C. In one embodiment, the data storage nucleic acid molecules comprised in the library are stored at a temperature comprised between 4° C. and −80° C. In one embodiment, the data storage nucleic acid molecules comprised in the library are stored at a temperature comprised between 4° C. and −20° C. In one embodiment, the data storage nucleic acid molecules comprised in the library are stored at a temperature comprised between 4° C. and 0° C. In one embodiment, the data storage nucleic acid molecules comprised in the library are stored at a temperature comprised between 0° C. and −200° C. In one embodiment, the data storage nucleic acid molecules comprised in the library are stored at a temperature comprised between −20° C. and −200° C. In one embodiment, the data storage nucleic acid molecules comprised in the library are stored at a temperature comprised between −80° C. and −200° C. In one embodiment, the data storage nucleic acid molecules comprised in the library are stored at a temperature of −196° C.

In one embodiment, the data storage nucleic acid molecules comprised in the library are stored in a suitable solvent. Suitable solvents for nucleic acid storage are known in the art. Non limitative examples of solvents used for nucleic acid storage include aqueous solvents such as demineralized water or biological buffers (e.g., phosphate-buffered saline, Tris-HCl).

In one embodiment, the data storage nucleic acid molecules comprised in the library are lyophilized.

The present invention further relates to a nucleic acid-based data storage system comprising at least two libraries according to the invention.

In one embodiment, the data storage nucleic acid molecules of the at least two libraries comprise bioblocks (e.g., biooctets) and/or components. In one embodiment, the data storage nucleic acid molecules of the at least two libraries comprise bioblocks (e.g., biooctets).

In one embodiment, the nucleic acid-based data storage system is for storing data comprised in a digital sequence as described hereinabove. In one embodiment, the conversion of information carried by the digital sequence into the nucleic acid-based data storage system, i.e., encoding, is performed using the method of the present disclosure.

In one embodiment, the digital data consist of binary digital data. In practice, converting digital data into a nucleic acid molecule may be performed automatically by a suitable software in silico.

In one embodiment, the data comprised in a digital sequence is stored on at least one data storage nucleic acid molecule, wherein the at least one data storage nucleic acid molecule is assembled using the method according to the invention, from libraries according to the invention.

In one embodiment, nucleic acid-based data storage system can store the equivalent of an amount of information comprised from 2 to 10²¹ bytes. As used herein, the expression “from 2 to 10²¹ bytes” comprises 1, 2, 3, 4, 5, 6, 7, 8, 9, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 128, 256, 500, 512, 1000, 1024, 2048, 4096, 8192, 10⁴, 10⁵, 10⁶, 10⁷, 10⁸, 10⁹, 10¹⁰, 10¹¹, 10¹², 10¹³, 10¹⁴, 10¹⁵, 10¹⁶, 10¹⁷, 10¹⁸, 10¹⁹, 10²⁰, 10²¹ bytes.

Another object of the present invention is a computer software for implementing the use and method for storing digital data.

In one embodiment, the method of the invention is implemented with a microprocessor comprising a software configured to assign to digital data at least one nucleic acid molecule according to the invention. In some embodiments, the software is configured to prevent that the sequence of the composite nucleic acid molecule according to the invention would encode one or more RNA(s), preferably would not encode any mRNA(s). In some embodiments, the software is configured to prevent that the sequence of the composite nucleic acid molecule according to the invention would comprise one or more initiation codon(s) in all 6 reading frames. In some embodiments, the software is configured to prevent that the sequence of the composite nucleic acid molecule according to the invention would comprise one or more specific restriction site(s). In some embodiments, the software is configured to prevent that the sequence of the composite nucleic acid molecule according to the invention would comprise one or more repeat(s) of at least 5 identical nucleotides.

In one embodiment, information can be retrieved from the nucleic acid-based data storage system by sequencing the at least one nucleic acid molecule. Methods of sequencing nucleic acid molecules, in particular high throughput sequencing, are known in the art and comprise, inter alia, Illumina (sequencing by synthesis), single-molecule real-time (SMRT) sequencing, nanopore sequencing (e.g., sequencing solutions from Oxford Nanopore Technologies), sequencing by ligation or sequencing by chain termination (Sanger method).

In one embodiment, converting the data retrieved from the data storage system into digital data further requires to obtain:
the encoding system used to convert digital subsequences comprising m bits (i.e., value and position of the bits) into bioblocks, according to the method of the invention,
the sequence of the regions comprising cleavage sites,
the position and type of metadata bioblocks,
the value of m,
the value of n and x.

In one embodiment, converting the data retrieved from the data storage system into digital data results in the retrieval of a sequence of bytes comprising m bits. In one embodiment, converting the data retrieved from the data storage system into digital data results in the retrieval of a sequence of octets.

In one embodiment, the information required to convert the data retrieved from the data storage system into digital data is stored in at least one database. In another embodiment, the information required to convert the data retrieved from the data storage system into digital data is stored in metadata bioblocks.

In one embodiment, the conversion of data contained in the data storage system into digital data is automated, i.e., by a suitable software or program. In practice, a program in which are entered (i) the sequence of the at least one data storage nucleic acid molecule and (ii) the information required to convert the data retrieved from the data storage system into digital data (i.e., the encoding system, the sequence of the cleavage sites, the position and type of metadata bioblocks, the value of m and the value of both n and x), provides a sequence of bytes comprising m bits, optionally a sequence of octets. Typically, the nucleotides corresponding to the cleavage sites are skipped by the program.

In one embodiment, said sequence of bytes, optionally octets, is read as such. In one embodiment, said sequence of bytes, optionally octets, is first converted to a file format as described in the present disclosure. In one embodiment, the converted file is read by an adequate program.

Another object of the present invention is a computer software for implementing the use and method for retrieving digital data. In one embodiment, the method of the invention is implemented with a microprocessor comprising a software configured to convert at least one nucleic acid sequence into digital data, using the method as described hereinabove.

### BRIEF DESCRIPTION OF THE DRAWINGS

FIG. 1 is a schematic representing the pipeline of the complete method for encoding digital data.

FIG. 2 is a schematic representing the design of library A and library B, with each plasmid comprising one bioblock.

FIG. 3 is a schematic representing the design of BioblockX2 plasmids.

FIG. 4 is a schematic representing the design of BioblockX64 plasmids.

FIG. 5 is a schematic representing the design of BioblockX1024 plasmids.

### EXAMPLES

The present invention is further illustrated by the following example of 8-bit biodata encoding

### Materials and Methods

Practical biodata encoding of a text file containing the poem Liberté written by Paul Eluard in 1942 (Table 1).

TABLE 1
Original text, poem Liberté by Paul Eluard

Liberté
Sur mes cahiers d'écolier
Sur mon pupitre et les arbres
Sur le sable sur la neige
J'écris ton nom
Sur toutes les pages lues
Sur toutes les pages blanches
Pierre sang papier ou cendre
J'écris ton nom
Sur les images dorées
Sur les armes des guerriers
Sur la couronne des rois
J'écris ton nom
Sur la jungle et le désert
Sur les nids sur les genêts
Sur l'echo de mon enfance
J'écris ton nom
Sur les merveilles des nuits
Sur le pain blanc des journées
Sur les saisons fiancées
J'écris ton nom
Sur tous mes chiffons d'azur
Sur l'étang soleil moisi
Sur le lac lune vivante
J'écris ton nom
Sur les champs sur l'horizon
Sur les ailes des oiseaux
Et sur le moulin des ombres
J'écris ton nom
Sur chaque bouffée d'aurore
Sur la mer sur les bateaux
Sur la montagne démente
J'écris ton nom
Sur la mousse des nuages
Sur les sueurs de l'orage
Sur la pluie épaisse et fade
J'écris ton nom
Sur les formes scintillantes
Sur les cloches des couleurs
Sur la vérité physique
J'écris ton nom
Sur les sentiers éveillés
Sur les routes déployées
Sur les places qui débordent
J'écris ton nom
Sur la lampe qui s'allume
Sur la lampe qui s'éteint
Sur mes maisons réunies
J'écris ton nom
Sur le fruit coupé en deux
Du miroir et de ma chambre
Sur mon lit coquille vide
J'écris ton nom
Sur mon chien gourmand et tendre
Sur ses oreilles dressées
Sur sa patte maladroite
J'écris ton nom
Sur le tremplin de ma porte
Sur les objets familiers
Sur le flot du feu béni
J'écris ton nom
Sur toute chair accordée
Sur le front de mes amis
Sur chaque main qui se tend
J'écris ton nom
Sur la vitre des surprises
Sur les lèvres attentives
Bien au-dessus du silence
J'écris ton nom
Sur mes refuges détruits
Sur mes phares écroulés
Sur les murs de mon ennui
J'écris ton nom
Sur l'absence sans désir
Sur la solitude nue
Sur les marches de la mort
J'écris ton nom
Sur la santé revenue
Sur le risque disparu
Sur l'espoir sans souvenir
J'écris ton nom
Et par le pouvoir d'un mot
Je recommence ma vie
Je suis né pour te connâitre
Pour te nommer
Liberté.
Paul Eluard
***
Encodé par le Centre National de la Recherche Scientifique et
Sorbonne Université à Paris, France, 2021.
Avec la permission des Éditions de Minuit.

The text is encoded using the ISO8859-1 standard, also known as Latin-1, to generate file A comprising 2358 octets (Table 2). File A is compressed as a 7z archive with the LZMA2 algorithm to generate file B comprising 1137 octets (Table 3). File B corresponds to a digital sequence formed of a plurality of 9096 bits. This digital sequence is subdivided into n=1137 digital subsequences each comprising m=8 bits. Each of these 1137 digital subsequences of 8 bits are converted into a bioblock of m=8 nucleotides named a biooctet.

TABLE 2
File A, ISO8859-1 encoding of the original text, 2358 octets

0100110001101001011000100110010101110010011101001110100100001101000010100000110100
0010100000110100001010010100110111010101110010001000000110110101100101011100110010
0000011000110110000101101000011010010110010101110010011100110010000001100100001001
1111101001011000110110111101101100011010010110010101110010000011010000101001010011
0111010101110010001000000110110101101111011011100010000001110000011101010111000001
1010010111010001110010011001010010000001100101011101000010000001101100011001010111
0011001000000110000101110010011000100111001001100101011100110000110100001010010100
1101110101011100100010000001101100011001010010000001110011011000010110001001101100
0110010100100000011100110111010101110010001000000110110001100001001000000110111001
1001010110100101100111011001010000110100001010010010100010011111101001011000110111
0010011010010111001100100000011101000110111101101110001000000110111001101111011011
0100001101000010100000110100001010000011010000101001010011011101010111001000100000
0111010001101111011101010111010001100101011100110010000001101100011001010111001100
1000000111000001100001011001110110010101110011001000000110110001110101011001010111
0011000011010000101001010011011101010111001000100000011101000110111101110101011101
0001100101011100110010000001101100011001010111001100100000011100000110000101100111
0110010101110011001000000110001001101100011000010110111001100011011010000110010101
1100110000110100001010010100000110100101100101011100100111001001100101001000000111
0011011000010110111001100111001000000111000001100001011100000110100101100101011100
1000100000011011110111010100100000011000110110010101101110011001000111001001100101
0000110100001010010010100010011111101001011000110111001001101001011100110010000001
1101000110111101101110001000000110111001101111011011010000110100001010000011010000
1010000011010000101001010011011101010111001000100000011011000110010101110011001000
0001101001011011010110000101100111011001010111001100100000011001000110111101110010
1110100101100101011100110000110100001010010100110111010101110010001000000110110001
1001010111001100100000011000010111001001101101011001010111001100100000011001000110
0101011100110010000001100111011101010110010101110010011100100110100101100101011100
1001110011000011010000101001010011011101010111001000100000011011000110000100100000
0110001101101111011101010111001001101111011011100110111001100101001000000110010001
1001010111001100100000011100100110111101101001011100110000110100001010010010100010
0111111010010110001101110010011010010111001100100000011101000110111101101110001000
0001101110011011110110110100001101000010100000110100001010000011010000101001010011
0111010101110010001000000110110001100001001000000110101001110101011011100110011101
1011000110010100100000011001010111010000100000011011000110010100100000011001001110
1001011100110110010101110010011101000000110100001010010100110111010101110010001000
0001101100011001010111001100100000011011100110100101100100011100110010000001110011
0111010101110010001000000110110001100101011100110010000001100111011001010110111011
1010100111010001110011000011010000101001010011011101010111001000100000011011000010
0111111010010110001101101000011011110010000001100100011001010010000001101101011011
1101101110001000000110010101101110011001100110000101101110011000110110010100001101
0000101001001010001001111110100101100011011100100110100101110011001000000111010001
1011110110111000100000011011100110111101101101000011010000101000001101000010100000
1101000010100101001101110101011100100010000001101100011001010111001100100000011011
0101100101011100100111011001100101011010010110110001101100011001010111001100100000
0110010001100101011100110010000001101110011101010110100101110100011100110000110100
0010100101001101110101011100100010000001101100011001010010000001110000011000010110
1001011011100010000001100010011011000110000101101110011000110010000001100100011001
0101110011001000000110101001101111011101010111001001101110111010010110010101110011
0000110100001010010100110111010101110010001000000110110001100101011100110010000001
1100110110000101101001011100110110111101101110011100110010000001100110011010010110
0001011011100110001111101001011001010111001100001101000010100100101000100111111010
0101100011011100100110100101110011001000000111010001101111011011100010000001101110
0110111101101101000011010000101000001101000010100000110100001010010100110111010101
1100100010000001110100011011110111010101110011001000000110110101100101011100110010
0000011000110110100001101001011001100110011001101111011011100111001100100000011001
0000100111011000010111101001110101011100100000110100001010010100110111010101110010
0010000001101100001001111110100101110100011000010110111001100111001000000111001101
1011110110110001100101011010010110110000100000011011010110111101101001011100110110
1001000011010000101001010011011101010111001000100000011011000110010100100000011011
0001100001011000110010000001101100011101010110111001100101001000000111011001101001
0111011001100001011011100111010001100101000011010000101001001010001001111110100101
1000110111001001101001011100110010000001110100011011110110111000100000011011100110
1111011011010000110100001010000011010000101000001101000010100101001101110101011100
1000100000011011000110010101110011001000000110001101101000011000010110110101110000
0111001100100000011100110111010101110010001000000110110000100111011010000110111101
1100100110100101111010011011110110111000001101000010100101001101110101011100100010
0000011011000110010101110011001000000110000101101001011011000110010101110011001000
0001100100011001010111001100100000011011110110100101110011011001010110000101110101
0111100000001101000010100100010101110100001000000111001101110101011100100010000001
1011000110010100100000011011010110111101110101011011000110100101101110001000000110
0100011001010111001100100000011011110110110101100010011100100110010101110011000011
0100001010010010100010011111101001011000110111001001101001011100110010000001110100
0110111101101110001000000110111001101111011011010000110100001010000011010000101000
0011010000101001010011011101010111001000100000011000110110100001100001011100010111
0101011001010010000001100010011011110111010101100110011001101110100101100101001000
0001100100001001110110000101110101011100100110111101110010011001010000110100001010
0101001101110101011100100010000001101100011000010010000001101101011001010111001000
1000000111001101110101011100100010000001101100011001010111001100100000011000100110
0001011101000110010101100001011101010111100000001101000010100101001101110101011100
1000100000011011000110000100100000011011010110111101101110011101000110000101100111
0110111001100101001000000110010011101001011011010110010101101110011101000110010100
0011010000101001001010001001111110100101100011011100100110100101110011001000000111
0100011011110110111000100000011011100110111101101101000011010000101000001101000010
1000001101000010100101001101110101011100100010000001101100011000010010000001101101
0110111101110101011100110111001101100101001000000110010001100101011100110010000001
1011100111010101100001011001110110010101110011000011010000101001010011011101010111
0010001000000110110001100101011100110010000001110011011101010110010101110101011100
1001110011001000000110010001100101001000000110110000100111011011110111001001100001
0110011101100101000011010000101001010011011101010111001000100000011011000110000100
1000000111000001101100011101010110100101100101001000001110100101110000011000010110
1001011100110111001101100101001000000110010101110100001000000110011001100001011001
0001100101000011010000101001001010001001111110100101100011011100100110100101110011
0010000001110100011011110110111000100000011011100110111101101101000011010000101000
0011010000101000001101000010100101001101110101011100100010000001101100011001010111
0011001000000110011001101111011100100110110101100101011100110010000001110011011000
1101101001011011100111010001101001011011000110110001100001011011100111010001100101
0111001100001101000010100101001101110101011100100010000001101100011001010111001100
1000000110001101101100011011110110001101101000011001010111001100100000011001000110
0101011100110010000001100011011011110111010101101100011001010111010101110010011100
1100001101000010100101001101110101011100100010000001101100011000010010000001110110
1110100101110010011010010111010011101001001000000111000001101000011110010111001101
1010010111000101110101011001010000110100001010010010100010011111101001011000110111
0010011010010111001100100000011101000110111101101110001000000110111001101111011011
0100001101000010100000110100001010000011010000101001010011011101010111001000100000
0110110001100101011100110010000001110011011001010110111001110100011010010110010101
1100100111001100100000111010010111011001100101011010010110110001101100111010010111
0011000011010000101001010011011101010111001000100000011011000110010101110011001000
0001110010011011110111010101110100011001010111001100100000011001001110100101110000
0110110001101111011110011110100101100101011100110000110100001010010100110111010101
1100100010000001101100011001010111001100100000011100000110110001100001011000110110
0101011100110010000001110001011101010110100100100000011001001110100101100010011011
1101110010011001000110010101101110011101000000110100001010010010100010011111101001
0110001101110010011010010111001100100000011101000110111101101110001000000110111001
1011110110110100001101000010100000110100001010000011010000101001010011011101010111
0010001000000110110001100001001000000110110001100001011011010111000001100101001000
0001110001011101010110100100100000011100110010011101100001011011000110110001110101
0110110101100101000011010000101001010011011101010111001000100000011011000110000100
1000000110110001100001011011010111000001100101001000000111000101110101011010010010
0000011100110010011111101001011101000110010101101001011011100111010000001101000010
1001010011011101010111001000100000011011010110010101110011001000000110110101100001
0110100101110011011011110110111001110011001000000111001011101001011101010110111001
1010010110010101110011000011010000101001001010001001111110100101100011011100100110
1001011100110010000001110100011011110110111000100000011011100110111101101101000011
0100001010000011010000101000001101000010100101001101110101011100100010000001101100
0110010100100000011001100111001001110101011010010111010000100000011000110110111101
1101010111000011101001001000000110010101101110001000000110010001100101011101010111
1000000011010000101001000100011101010010000001101101011010010111001001101111011010
0101110010001000000110010101110100001000000110010001100101001000000110110101100001
0010000001100011011010000110000101101101011000100111001001100101000011010000101001
0100110111010101110010001000000110110101101111011011100010000001101100011010010111
0100001000000110001101101111011100010111010101101001011011000110110001100101001000
0001110110011010010110010001100101000011010000101001001010001001111110100101100011
0111001001101001011100110010000001110100011011110110111000100000011011100110111101
1011010000110100001010000011010000101000001101000010100101001101110101011100100010
0000011011010110111101101110001000000110001101101000011010010110010101101110001000
0001100111011011110111010101110010011011010110000101101110011001000010000001100101
0111010000100000011101000110010101101110011001000111001001100101000011010000101001
0100110111010101110010001000000111001101100101011100110010000001101111011100100110
0101011010010110110001101100011001010111001100100000011001000111001001100101011100
1101110011111010010110010101110011000011010000101001010011011101010111001000100000
0111001101100001001000000111000001100001011101000111010001100101001000000110110101
1000010110110001100001011001000111001001101111011010010111010001100101000011010000
1010010010100010011111101001011000110111001001101001011100110010000001110100011011
1101101110001000000110111001101111011011010000110100001010000011010000101000001101
0000101001010011011101010111001000100000011011000110010100100000011101000111001001
1001010110110101110000011011000110100101101110001000000110010001100101001000000110
1101011000010010000001110000011011110111001001110100011001010000110100001010010100
1101110101011100100010000001101100011001010111001100100000011011110110001001101010
0110010101110100011100110010000001100110011000010110110101101001011011000110100101
1001010111001001110011000011010000101001010011011101010111001000100000011011000110
0101001000000110011001101100011011110111010000100000011001000111010100100000011001
1001100101011101010010000001100010111010010110111001101001000011010000101001001010
0010011111101001011000110111001001101001011100110010000001110100011011110110111000
1000000110111001101111011011010000110100001010000011010000101000001101000010100101
0011011101010111001000100000011101000110111101110101011101000110010100100000011000
1101101000011000010110100101110010001000000110000101100011011000110110111101110010
0110010011101001011001010000110100001010010100110111010101110010001000000110110001
1001010010000001100110011100100110111101101110011101000010000001100100011001010010
0000011011010110010101110011001000000110000101101101011010010111001100001101000010
1001010011011101010111001000100000011000110110100001100001011100010111010101100101
0010000001101101011000010110100101101110001000000111000101110101011010010010000001
1100110110010100100000011101000110010101101110011001000000110100001010010010100010
0111111010010110001101110010011010010111001100100000011101000110111101101110001000
0001101110011011110110110100001101000010100000110100001010000011010000101001010011
0111010101110010001000000110110001100001001000000111011001101001011101000111001001
1001010010000001100100011001010111001100100000011100110111010101110010011100000111
0010011010010111001101100101011100110000110100001010010100110111010101110010001000
0001101100011001010111001100100000011011001110100001110110011100100110010101110011
0010000001100001011101000111010001100101011011100111010001101001011101100110010101
1100110000110100001010010000100110100101100101011011100010000001100001011101010010
1101011001000110010101110011011100110111010101110011001000000110010001110101001000
0001110011011010010110110001100101011011100110001101100101000011010000101001001010
0010011111101001011000110111001001101001011100110010000001110100011011110110111000
1000000110111001101111011011010000110100001010000011010000101000001101000010100101
0011011101010111001000100000011011010110010101110011001000000111001001100101011001
1001110101011001110110010101110011001000000110010011101001011101000111001001110101
0110100101110100011100110000110100001010010100110111010101110010001000000110110101
1001010111001100100000011100000110100001100001011100100110010101110011001000001110
1001011000110111001001101111011101010110110011101001011100110000110100001010010100
1101110101011100100010000001101100011001010111001100100000011011010111010101110010
0111001100100000011001000110010100100000011011010110111101101110001000000110010101
1011100110111001110101011010010000110100001010010010100010011111101001011000110111
0010011010010111001100100000011101000110111101101110001000000110111001101111011011
0100001101000010100000110100001010000011010000101001010011011101010111001000100000
0110110000100111011000010110001001110011011001010110111001100011011001010010000001
1100110110000101101110011100110010000001100100111010010111001101101001011100100000
1101000010100101001101110101011100100010000001101100011000010010000001110011011011
1101101100011010010111010001110101011001000110010100100000011011100111010101100101
0000110100001010010100110111010101110010001000000110110001100101011100110010000001
1011010110000101110010011000110110100001100101011100110010000001100100011001010010
0000011011000110000100100000011011010110111101110010011101000000110100001010010010
1000100111111010010110001101110010011010010111001100100000011101000110111101101110
0010000001101110011011110110110100001101000010100000110100001010000011010000101001
0100110111010101110010001000000110110001100001001000000111001101100001011011100111
0100111010010010000001110010011001010111011001100101011011100111010101100101000011
0100001010010100110111010101110010001000000110110001100101001000000111001001101001
0111001101110001011101010110010100100000011001000110100101110011011100000110000101
1100100111010100001101000010100101001101110101011100100010000001101100001001110110
0101011100110111000001101111011010010111001000100000011100110110000101101110011100
1100100000011100110110111101110101011101100110010101101110011010010111001000001101
0000101001001010001001111110100101100011011100100110100101110011001000000111010001
1011110110111000100000011011100110111101101101000011010000101000001101000010100000
1101000010100100010101110100001000000111000001100001011100100010000001101100011001
0100100000011100000110111101110101011101100110111101101001011100100010000001100100
0010011101110101011011100010000001101101011011110111010000001101000010100100101001
1001010010000001110010011001010110001101101111011011010110110101100101011011100110
0011011001010010000001101101011000010010000001110110011010010110010100001101000010
1001001010011001010010000001110011011101010110100101110011001000000110111011101001
0010000001110000011011110111010101110010001000000111010001100101001000000110001101
1011110110111001101110011000011110111001110100011100100110010100001101000010100101
0000011011110111010101110010001000000111010001100101001000000110111001101111011011
0101101101011001010111001000001101000010100000110100001010000011010000101001001100
0110100101100010011001010111001001110100111010010010111000001101000010100000110100
0010100000110100001010010100000110000101110101011011000010000001000101011011000111
0101011000010111001001100100000011010000101000001101000010100000110100001010001010
1000101010001010100010101000101010000011010000101001000101011011100110001101101111
0110010011101001001000000111000001100001011100100010000001101100011001010010000001
0000110110010101101110011101000111001001100101001000000100111001100001011101000110
1001011011110110111001100001011011000010000001100100011001010010000001101100011000
0100100000010100100110010101100011011010000110010101110010011000110110100001100101
0010000001010011011000110110100101100101011011100111010001101001011001100110100101
1100010111010101100101001000000110010101110100001000000101001101101111011100100110
0010011011110110111001101110011001010010000001010101011011100110100101110110011001
0101110010011100110110100101110100111010010010000011100000001000000101000001100001
0111001001101001011100110010110000100000010001100111001001100001011011100110001101
1001010010110000100000001100100011000000110010001100010010111000001101000010100100
0001011101100110010101100011001000000110110001100001001000000111000001100101011100
1001101101011010010111001101110011011010010110111101101110001000000110010001100101
0111001100100000110010010110010001101001011101000110100101101111011011100111001100
1000000110010001100101001000000100110101101001011011100111010101101001011101000010
1110

TABLE 3
File B, 7z archive of file A with LZMA2 compression, 1137 octets

0011011101111010101111001010111100100111000111000000000000000100110101001101111111
1110011011110011101111000000110000000000000000000000000000000000000000000000000110
0010000000000000000000000000000000000000000000000000000000001101100001001110100101
0010011111111000000000100100110101000000111110011101011101000000000010011000011010
0100100001000110011100011110101000100001001111100100100110101111111011111110000110
1111100110000011010001000100100111001101111100100001100000010001000000000000101110
1111110101111010001110011000100010100000001111011110111101100000110101011100101001
0110011001110100000001110101110011001111110001000011111100001000001011001101100110
0000010000101010010000011010000111101100000001000000011110101011100110111000011001
1001101110110010100011101010110011010110100001110110100001101000000100000011110010
1011010010111001100000110101110001001101000111100110011000100001000111100110101010
1111101001011111010101101000101111101110100011010110011110000011011010011000101101
0111011100010001000000110001010011100011110010110101011010001011000111111110011100
1110011111100110110010001010100111110100100101010000001100011100111000001011110111
0101100111011001111101100001000111010111100010111001100110111010111000110111010000
0011110101100001100011110111000011010110011001011011100000110010100110101010010111
0011011110100010111001010111000111110011011010011111101011100100001010111001000111
1101110010011110111101110100011101011001010011010111111010010001010101010101110000
0110010110101001011011001101011011111111011000000001001010000100000111111110110010
1010111001001110101110111011101010011110010011000110101101110100100111000001100011
1001101110001110011111001111000101111101100111001101010101000101000100001101011100
1101000001100011011011001110011100101110101011110100001001010001111100010000010111
0110110001011011100010101000101100101101011111111000100100011111111101010010011101
0101101110001010011010010011101101001001110100001010000110111110000000110100101101
1001011011110000110100010111100000010111110110110111000110010100001011010011111110
1100001111000110010001010011000110010000100100010001011110100011000111011100011101
0100101000111111100110000011111010110000111101101001101001000100001001111110010101
0010101001011000011111010111100111101100101111111001100001100000100101000000001100
0010001001110101010001111101001011011101101101110111110110110101000111100101001110
1001110010110110111110110100101001000001110110000010011111011001101000010011011101
0010110001111011101001110110010101000010101100010100000001000101000101101110111011
0101011101011000011101010111100011101111010010000011010100111110011100110100110001
0101110111010100000101010000010011011000111101001011011011001011001000100000110110
0000011010100001001100010100011101011010100110000101010100101111001101001000101000
1100111010001111000010000000101111101000110110101101101101000000011110001100010001
0000111100000000101010110000000110000000000001010010000001000101010001100001001110
1001001110000000001100011101111000001001001000101101010110000001101110010001011111
1001101010100110110101100101100011111101101000000111011101010101010101110001000100
1101100100000111010111100111010000000110101000111101101000101001001011001101110010
0110000111110101011110011011111011100101100101110110010110001011011011010000001000
1111010011100001101100110001011000010011110011101010011011011000110100000011110010
1110110010000011100111010111011000000010001011110010101100111000011111000000011011
0101111110011011010000111000001111010111010011100000001000101111110110100110000000
0000011110110101101000101100111110111111010011000110000100110111011111011110100100
1111110100100000001100100000000110011000011110001000101010001100001001101011110100
1000000101001100011101101000010011111001101101011011001001101110101011011110101101
1101100100010011010111110100011001011100100101011010101110001110100111111101011010
1001001110110100111100100111000010000100110011100010001001000010111110111111101010
0000111011000111101010110001101110000101110010100011001100011011110100000111011101
1011011010010010111111111101111000111001001110011010100110010010000001100010100110
1111010111011100101100001110011110101100110111101011001000011101101111001111101101
0101100000100110110000001010101101110111100010000001101100001100001001110111001000
1000000010111111101101111100001111101011100100000011100000100111001100011100010110
1011010010010101010001000100011101110011011000011001111100110011100001000101001101
1100010100001110000110010001010110011010000110101011000110011111001111101001100010
0100110001001110101001010101100001111000101111111111011000110101101101101100001001
0011111100111111100010100000011101010000010000101011111111101011101001010110001110
0100011100011001101111001000111111000100011010010000110001101101001000001111010000
1110100110010101011001001101101110110011111000001110011011000101000101110111010100
1001110101011101010000000111000110001010111100000011111010111111010000111100010111
0000100011100111011010110100100110111100101101100110110000010010010010010011111010
0100010010000010110011011001010001100111000101111010100111110000011100011000110111
1100001001110011010101110111111101010100010111110000010001011001111100110000000001
0101001111000000011100110100000000011100010001000110100111101010100101001011111101
1111001111011111010110010010111010111011000101001111110001100010010010010001100101
0011110011011001011011111110111001111110000111000101001001111001101010000101010001
1100010100010111000001101100110001011100010111110010110011001001011010001101001101
0100101011101000011110101111101100001110111100010010110101010101000101100111101101
1101101100101101000111101010010101010111101111000111011100011000011001110000001100
0010110011101110011101111101111100111101111001101011010010010011000111011000010010
1000100110110000101110110101100011101111100011010001110110111110111110001001111111
0100011101001100101000100011000100101010011101100100011100100111011101110110101110
1100101001010001111110001110001110010001011100111010000011111100011100010101000111
1001000110111011010100010010100111000111001101100111010001111011000110000010000011
0011111110000111010011110110010000000100000011101100111111101001011110110110010001
1001001000111110100000001010110001110000101100000001000111101101000111000101001000
1001001000010111100110110110011000011010110100111000001110010000111110000001001011
1000111111101011011110100110110110011111001001111110110010100001100111100101011100
0001000001010111011010001000100010011000100010010001000010110011000011000111001000
1111100001000000010000101011101011011111001000100011010111010101010111000000110010
0101111111000000111111111001110110000100010010000000110100110100100001100000111101
1100000111010110001110000110111111101110110001100001010000110010010010000000100000
0101010011110110100101111010000011000100001101010110000110001111100100000011100011
1011100011101010011011011101110110011100001000101000001111000001011110000101000111
0011011110011101000000110001100101010100010001010111101100111100111011001111000100
0110000101100000110001110100001001001111000000011000110110101010001110000100010101
1101000011100100000100110101101000000001100000001101001000101001110110110011001001
0100000111110101000100110110011010010010111011111000111000001100010000010100000101
1000101010010111011110011110110101110101101001001001000101110111101100110011001001
1010000101011001110110000001000111000110101101001011010100101000010011100110011010
0010101011100000011011001011100011100000000011101110000110000001010110111111010110
1101111101011000011000110000111001111001010001010110110100100000011001000111011110
0000110010011000001100110010110111101101001011000001001111111101010110110100111011
0001111100100000100110010100000100111111011111000111010011100001101101111011010011
0101000011010110001010011111100010001101001001100110101111011011011100001110010101
1010101011111110001001000010001101011101010001110000010100010011010111001110001001
1100001000011001000101010101101111101001111010011010101000011101010010101110000000
0010001110000111000001011101001000011010101101111110000110001010110111010100101101
0111001111111000011011111000100001011001101010001111101101100100111010000100010111
0000110110000000100001011011000111111100010111011010010111110000000011100100000100
1010011001011100110001100111111000110100110001001111100100111011011010011111011000
1001000100111001011010000000000000000100000100000001100000000000000001000010011000
0011111011110000000000000111000010110000000100000000000000010010000100100001000000
0100000000000011001000100100110110000000000000100000001010000000010000101001001100
0000001000011111000000000000000000000101000000010001100100001010000000000000000000
0000000000000000000000000000000000000000000000000000000000000000010001000110010000
0000010011000000000001101001000000000110001000000000011001010000000001110010000000
0001110100000000001110100100000000001011100000000001110100000000000111100000000000
0111010000000000000000000000000000011001000000100000000000000000000101000000101000
0000010000000000000000111101111110011010110011011000110011111111011000000000010001
0101000001100000000100000000001000001000000010110100100000010000000000000000

For this conversion, the nucleotides are selected among four natural nucleotides: adenine (A), thymine (T), cytosine (C) and guanine (G). The conversion of each digital subsequence into a biooctet consists in converting bits 0 at even positions to nucleotide N1=A, bits 1 at even position to nucleotide N2=T, bits 0 at odd position to nucleotide N3=C and bits 1 at odd position to nucleotide N4=G.

The size of the longest assembly, called a track, was limited to 1024 biooctets. File B comprises more than 1024 biooctets and will therefore be assembled on more than one track. To be able to rearrange the tracks in the right order, a binary barcode was added, composed of four biooctets, at the beginning of each track. A total of 256 to the power of 4 (4 294 967 296) barcodes are available. The first track (track 0) contains barcode 0 composed of the 4 identical biooctets 0 of sequence “ACACACAC” (SEQ ID NO: 1107) followed by the first 1020 biooctets of file B. The second track contains barcode 1, composed of 3 octets 0 of sequence “ACACACAC” followed by one biooctet 1 of sequence “ACACACAG”, followed by the last 117 biooctets of file B. A last special biooctet named EOF_B of sequence “CAGTCTGT” is added at the end of track 1 to mark the end of the file (EOF). Therefore Track 0 contains 1024 biooctets and Track 1 contains 122 biooctets.

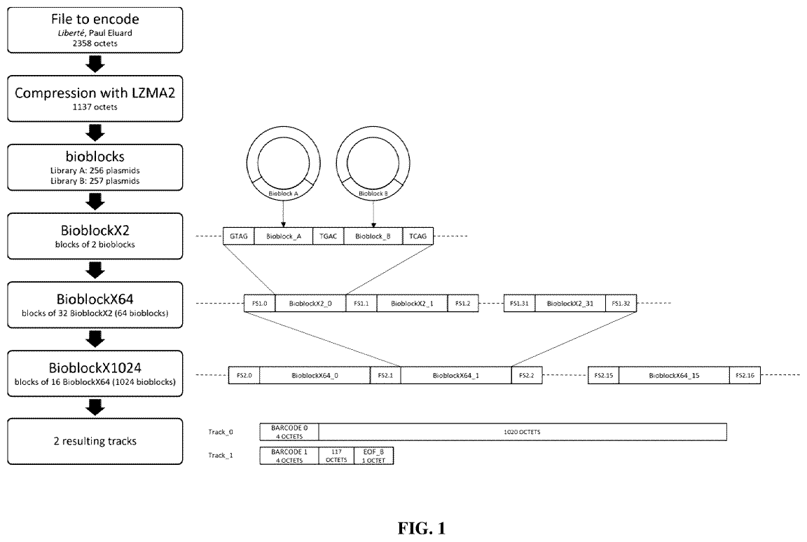

To generate the DNA molecules corresponding to the two tracks it is possible for example to perform three golden gate assembly steps to assemble the 1146 biooctets (FIG. 1). At step 1, the biooctets are assembled from two libraries containing all biooctets in blocks of 2 biooctets named BioblockX2. At step 2, blocks containing 32 BioblockX2 and named BioblockX64 are assembled. At step 3, blocks containing 16 BioblockX64 and named BioblockX1024 are assembled.

### Results

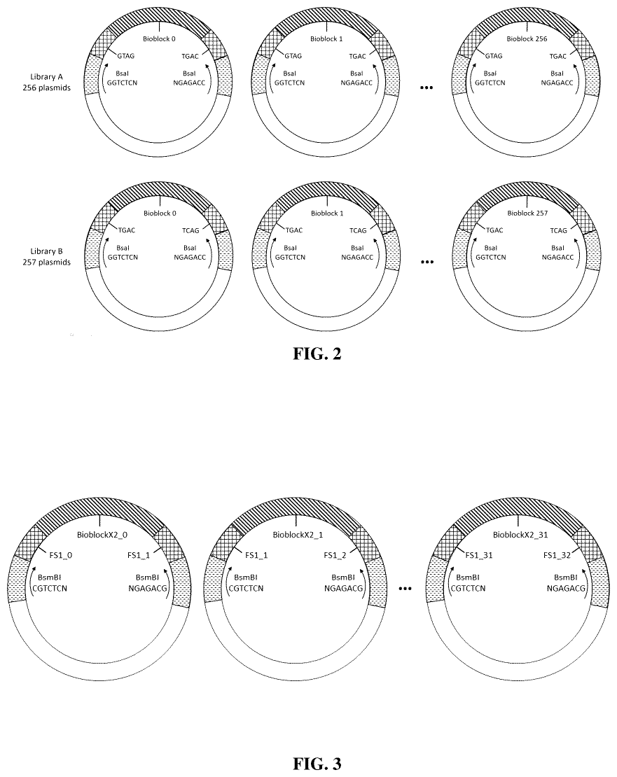

Two libraries named ‘library A’ and ‘library B’ and containing all the 256 possible biooctets are constructed. The EOF biooctet EOF_B is added to library B, which is therefore composed of 257 biooctets. In the two libraries, each biooctet is surrounded by regions comprising a BsaI cleavage site of 11 nucleotides and is contained in a double-stranded replicative plasmid. The variable region of the BsaI cleavage site, named fusion site, is defined for each library. In library A each biooctet is surrounded by the GTAG fusion site upstream of the biooctet and the TGAC fusion site downstream of the biooctet. In library B, each biooctet is surrounded by the TGAC fusion site upstream of the biooctet and the TCAG fusion site downstream of the biooctet. The composition of libraries A and B are provided in Table 4 and their design is presented in FIG. 2.

TABLE 4
Sequences of library A and library B bioblocks and their
surrounding fusion sites. The fusion sites are bolded.

| | | SEQ ID | | SEQ ID |
|---|---|---|---|---|
| Octets | Library A | NO: | Library B | NO: |
| 00000000 | GTAGACACACACTGAC | 594 | TGACACACACACTCAG | 850 |
| 00000001 | GTAGACACACAGTGAC | 595 | TGACACACACAGTCAG | 851 |
| 00000010 | GTAGACACACTCTGAC | 596 | TGACACACACTCTCAG | 852 |
| 00000011 | GTAGACACACTGTGAC | 597 | TGACACACACTGTCAG | 853 |
| 00000100 | GTAGACACAGACTGAC | 598 | TGACACACAGACTCAG | 854 |
| 00000101 | GTAGACACAGAGTGAC | 599 | TGACACACAGAGTCAG | 855 |
| 00000110 | GTAGACACAGTCTGAC | 600 | TGACACACAGTCTCAG | 856 |
| 00000111 | GTAGACACAGTGTGAC | 601 | TGACACACAGTGTCAG | 857 |
| 00001000 | GTAGACACTCACTGAC | 602 | TGACACACTCACTCAG | 858 |
| 00001001 | GTAGACACTCAGTGAC | 603 | TGACACACTCAGTCAG | 859 |
| 00001010 | GTAGACACTCTCTGAC | 604 | TGACACACTCTCTCAG | 860 |
| 00001011 | GTAGACACTCTGTGAC | 605 | TGACACACTCTGTCAG | 861 |
| 00001100 | GTAGACACTGACTGAC | 606 | TGACACACTGACTCAG | 862 |
| 00001101 | GTAGACACTGAGTGAC | 607 | TGACACACTGAGTCAG | 863 |
| 00001110 | GTAGACACTGTCTGAC | 608 | TGACACACTGTCTCAG | 864 |
| 00001111 | GTAGACACTGTGTGAC | 609 | TGACACACTGTGTCAG | 865 |
| 00010000 | GTAGACAGACACTGAC | 610 | TGACACAGACACTCAG | 866 |
| 00010001 | GTAGACAGACAGTGAC | 611 | TGACACAGACAGTCAG | 867 |
| 00010010 | GTAGACAGACTCTGAC | 612 | TGACACAGACTCTCAG | 868 |
| 00010011 | GTAGACAGACTGTGAC | 613 | TGACACAGACTGTCAG | 869 |
| 00010100 | GTAGACAGAGACTGAC | 614 | TGACACAGAGACTCAG | 870 |
| 00010101 | GTAGACAGAGAGTGAC | 615 | TGACACAGAGAGTCAG | 871 |
| 00010110 | GTAGACAGAGTCTGAC | 616 | TGACACAGAGTCTCAG | 872 |
| 00010111 | GTAGACAGAGTGTGAC | 617 | TGACACAGAGTGTCAG | 873 |
| 00011000 | GTAGACAGTCACTGAC | 618 | TGACACAGTCACTCAG | 874 |
| 00011001 | GTAGACAGTCAGTGAC | 619 | TGACACAGTCAGTCAG | 875 |
| 00011010 | GTAGACAGTCTCTGAC | 620 | TGACACAGTCTCTCAG | 876 |
| 00011011 | GTAGACAGTCTGTGAC | 621 | TGACACAGTCTGTCAG | 877 |
| 00011100 | GTAGACAGTGACTGAC | 622 | TGACACAGTGACTCAG | 878 |
| 00011101 | GTAGACAGTGAGTGAC | 623 | TGACACAGTGAGTCAG | 879 |
| 00011110 | GTAGACAGTGTCTGAC | 624 | TGACACAGTGTCTCAG | 880 |
| 00011111 | GTAGACAGTGTGTGAC | 625 | TGACACAGTGTGTCAG | 881 |
| 00100000 | GTAGACTCACACTGAC | 626 | TGACACTCACACTCAG | 882 |
| 00100001 | GTAGACTCACAGTGAC | 627 | TGACACTCACAGTCAG | 883 |
| 00100010 | GTAGACTCACTCTGAC | 628 | TGACACTCACTCTCAG | 884 |
| 00100011 | GTAGACTCACTGTGAC | 629 | TGACACTCACTGTCAG | 885 |
| 00100100 | GTAGACTCAGACTGAC | 630 | TGACACTCAGACTCAG | 886 |
| 00100101 | GTAGACTCAGAGTGAC | 631 | TGACACTCAGAGTCAG | 887 |
| 00100110 | GTAGACTCAGTCTGAC | 632 | TGACACTCAGTCTCAG | 888 |
| 00100111 | GTAGACTCAGTGTGAC | 633 | TGACACTCAGTGTCAG | 889 |
| 00101000 | GTAGACTCTCACTGAC | 634 | TGACACTCTCACTCAG | 890 |
| 00101001 | GTAGACTCTCAGTGAC | 635 | TGACACTCTCAGTCAG | 891 |
| 00101010 | GTAGACTCTCTCTGAC | 636 | TGACACTCTCTCTCAG | 892 |
| 00101011 | GTAGACTCTCTGTGAC | 637 | TGACACTCTCTGTCAG | 893 |
| 00101100 | GTAGACTCTGACTGAC | 638 | TGACACTCTGACTCAG | 894 |
| 00101101 | GTAGACTCTGAGTGAC | 639 | TGACACTCTGAGTCAG | 895 |
| 00101110 | GTAGACTCTGTCTGAC | 640 | TGACACTCTGTCTCAG | 896 |
| 00101111 | GTAGACTCTGTGTGAC | 641 | TGACACTCTGTGTCAG | 897 |
| 00110000 | GTAGACTGACACTGAC | 642 | TGACACTGACACTCAG | 898 |
| 00110001 | GTAGACTGACAGTGAC | 643 | TGACACTGACAGTCAG | 899 |
| 00110010 | GTAGACTGACTCTGAC | 644 | TGACACTGACTCTCAG | 900 |
| 00110011 | GTAGACTGACTGTGAC | 645 | TGACACTGACTGTCAG | 901 |
| 00110100 | GTAGACTGAGACTGAC | 646 | TGACACTGAGACTCAG | 902 |
| 00110101 | GTAGACTGAGAGTGAC | 647 | TGACACTGAGAGTCAG | 903 |
| 00110110 | GTAGACTGAGTCTGAC | 648 | TGACACTGAGTCTCAG | 904 |
| 00110111 | GTAGACTGAGTGTGAC | 649 | TGACACTGAGTGTCAG | 905 |
| 00111000 | GTAGACTGTCACTGAC | 650 | TGACACTGTCACTCAG | 906 |
| 00111001 | GTAGACTGTCAGTGAC | 651 | TGACACTGTCAGTCAG | 907 |
| 00111010 | GTAGACTGTCTCTGAC | 652 | TGACACTGTCTCTCAG | 908 |
| 00111011 | GTAGACTGTCTGTGAC | 653 | TGACACTGTCTGTCAG | 909 |
| 00111100 | GTAGACTGTGACTGAC | 654 | TGACACTGTGACTCAG | 910 |
| 00111101 | GTAGACTGTGAGTGAC | 655 | TGACACTGTGAGTCAG | 911 |
| 00111110 | GTAGACTGTGTCTGAC | 656 | TGACACTGTGTCTCAG | 912 |
| 00111111 | GTAGACTGTGTGTGAC | 657 | TGACACTGTGTGTCAG | 913 |
| 01000000 | GTAGAGACACACTGAC | 658 | TGACAGACACACTCAG | 914 |
| 01000001 | GTAGAGACACAGTGAC | 659 | TGACAGACACAGTCAG | 915 |
| 01000010 | GTAGAGACACTCTGAC | 660 | TGACAGACACTCTCAG | 916 |
| 01000011 | GTAGAGACACTGTGAC | 661 | TGACAGACACTGTCAG | 917 |
| 01000100 | GTAGAGACAGACTGAC | 662 | TGACAGACAGACTCAG | 918 |
| 01000101 | GTAGAGACAGAGTGAC | 663 | TGACAGACAGAGTCAG | 919 |
| 01000110 | GTAGAGACAGTCTGAC | 664 | TGACAGACAGTCTCAG | 920 |
| 01000111 | GTAGAGACAGTGTGAC | 665 | TGACAGACAGTGTCAG | 921 |
| 01001000 | GTAGAGACTCACTGAC | 666 | TGACAGACTCACTCAG | 922 |
| 01001001 | GTAGAGACTCAGTGAC | 667 | TGACAGACTCAGTCAG | 923 |
| 01001010 | GTAGAGACTCTCTGAC | 668 | TGACAGACTCTCTCAG | 924 |
| 01001011 | GTAGAGACTCTGTGAC | 669 | TGACAGACTCTGTCAG | 925 |
| 01001100 | GTAGAGACTGACTGAC | 670 | TGACAGACTGACTCAG | 926 |
| 01001101 | GTAGAGACTGAGTGAC | 671 | TGACAGACTGAGTCAG | 927 |
| 01001110 | GTAGAGACTGTCTGAC | 672 | TGACAGACTGTCTCAG | 928 |
| 01001111 | GTAGAGACTGTGTGAC | 673 | TGACAGACTGTGTCAG | 929 |
| 01010000 | GTAGAGAGACACTGAC | 674 | TGACAGAGACACTCAG | 930 |
| 01010001 | GTAGAGAGACAGTGAC | 675 | TGACAGAGACAGTCAG | 931 |
| 01010010 | GTAGAGAGACTCTGAC | 676 | TGACAGAGACTCTCAG | 932 |
| 01010011 | GTAGAGAGACTGTGAC | 677 | TGACAGAGACTGTCAG | 933 |
| 01010100 | GTAGAGAGAGACTGAC | 678 | TGACAGAGAGACTCAG | 934 |
| 01010101 | GTAGAGAGAGAGTGAC | 679 | TGACAGAGAGAGTCAG | 935 |
| 01010110 | GTAGAGAGAGTCTGAC | 680 | TGACAGAGAGTCTCAG | 936 |
| 01010111 | GTAGAGAGAGTGTGAC | 681 | TGACAGAGAGTGTCAG | 937 |
| 01011000 | GTAGAGAGTCACTGAC | 682 | TGACAGAGTCACTCAG | 938 |
| 01011001 | GTAGAGAGTCAGTGAC | 683 | TGACAGAGTCAGTCAG | 939 |
| 01011010 | GTAGAGAGTCTCTGAC | 684 | TGACAGAGTCTCTCAG | 940 |
| 01011011 | GTAGAGAGTCTGTGAC | 685 | TGACAGAGTCTGTCAG | 941 |
| 01011100 | GTAGAGAGTGACTGAC | 686 | TGACAGAGTGACTCAG | 942 |
| 01011101 | GTAGAGAGTGAGTGAC | 687 | TGACAGAGTGAGTCAG | 943 |
| 01011110 | GTAGAGAGTGTCTGAC | 688 | TGACAGAGTGTCTCAG | 944 |
| 01011111 | GTAGAGAGTGTGTGAC | 689 | TGACAGAGTGTGTCAG | 945 |
| 01100000 | GTAGAGTCACACTGAC | 690 | TGACAGTCACACTCAG | 946 |
| 01100001 | GTAGAGTCACAGTGAC | 691 | TGACAGTCACAGTCAG | 947 |
| 01100010 | GTAGAGTCACTCTGAC | 692 | TGACAGTCACTCTCAG | 948 |
| 01100011 | GTAGAGTCACTGTGAC | 693 | TGACAGTCACTGTCAG | 949 |
| 01100100 | GTAGAGTCAGACTGAC | 694 | TGACAGTCAGACTCAG | 950 |
| 01100101 | GTAGAGTCAGAGTGAC | 695 | TGACAGTCAGAGTCAG | 951 |
| 01100110 | GTAGAGTCAGTCTGAC | 696 | TGACAGTCAGTCTCAG | 952 |
| 01100111 | GTAGAGTCAGTGTGAC | 697 | TGACAGTCAGTGTCAG | 953 |
| 01101000 | GTAGAGTCTCACTGAC | 698 | TGACAGTCTCACTCAG | 954 |
| 01101001 | GTAGAGTCTCAGTGAC | 699 | TGACAGTCTCAGTCAG | 955 |
| 01101010 | GTAGAGTCTCTCTGAC | 700 | TGACAGTCTCTCTCAG | 956 |
| 01101011 | GTAGAGTCTCTGTGAC | 701 | TGACAGTCTCTGTCAG | 957 |
| 01101100 | GTAGAGTCTGACTGAC | 702 | TGACAGTCTGACTCAG | 958 |
| 01101101 | GTAGAGTCTGAGTGAC | 703 | TGACAGTCTGAGTCAG | 959 |
| 01101110 | GTAGAGTCTGTCTGAC | 704 | TGACAGTCTGTCTCAG | 960 |
| 01101111 | GTAGAGTCTGTGTGAC | 705 | TGACAGTCTGTGTCAG | 961 |
| 01110000 | GTAGAGTGACACTGAC | 706 | TGACAGTGACACTCAG | 962 |
| 01110001 | GTAGAGTGACAGTGAC | 707 | TGACAGTGACAGTCAG | 963 |
| 01110010 | GTAGAGTGACTCTGAC | 708 | TGACAGTGACTCTCAG | 964 |
| 01110011 | GTAGAGTGACTGTGAC | 709 | TGACAGTGACTGTCAG | 965 |
| 01110100 | GTAGAGTGAGACTGAC | 710 | TGACAGTGAGACTCAG | 966 |
| 01110101 | GTAGAGTGAGAGTGAC | 711 | TGACAGTGAGAGTCAG | 967 |
| 01110110 | GTAGAGTGAGTCTGAC | 712 | TGACAGTGAGTCTCAG | 968 |
| 01110111 | GTAGAGTGAGTGTGAC | 713 | TGACAGTGAGTGTCAG | 969 |
| 01111000 | GTAGAGTGTCACTGAC | 714 | TGACAGTGTCACTCAG | 970 |
| 01111001 | GTAGAGTGTCAGTGAC | 715 | TGACAGTGTCAGTCAG | 971 |
| 01111010 | GTAGAGTGTCTCTGAC | 716 | TGACAGTGTCTCTCAG | 972 |
| 01111011 | GTAGAGTGTCTGTGAC | 717 | TGACAGTGTCTGTCAG | 973 |
| 01111100 | GTAGAGTGTGACTGAC | 718 | TGACAGTGTGACTCAG | 974 |
| 01111101 | GTAGAGTGTGAGTGAC | 719 | TGACAGTGTGAGTCAG | 975 |
| 01111110 | GTAGAGTGTGTCTGAC | 720 | TGACAGTGTGTCTCAG | 976 |
| 01111111 | GTAGAGTGTGTGTGAC | 721 | TGACAGTGTGTGTCAG | 977 |
| 10000000 | GTAGTCACACACTGAC | 722 | TGACTCACACACTCAG | 978 |
| 10000001 | GTAGTCACACAGTGAC | 723 | TGACTCACACAGTCAG | 979 |
| 10000010 | GTAGTCACACTCTGAC | 724 | TGACTCACACTCTCAG | 980 |
| 10000011 | GTAGTCACACTGTGAC | 725 | TGACTCACACTGTCAG | 981 |
| 10000100 | GTAGTCACAGACTGAC | 726 | TGACTCACAGACTCAG | 982 |
| 10000101 | GTAGTCACAGAGTGAC | 727 | TGACTCACAGAGTCAG | 983 |
| 10000110 | GTAGTCACAGTCTGAC | 728 | TGACTCACAGTCTCAG | 984 |
| 10000111 | GTAGTCACAGTGTGAC | 729 | TGACTCACAGTGTCAG | 985 |
| 10001000 | GTAGTCACTCACTGAC | 730 | TGACTCACTCACTCAG | 986 |
| 10001001 | GTAGTCACTCAGTGAC | 731 | TGACTCACTCAGTCAG | 987 |
| 10001010 | GTAGTCACTCTCTGAC | 732 | TGACTCACTCTCTCAG | 988 |
| 10001011 | GTAGTCACTCTGTGAC | 733 | TGACTCACTCTGTCAG | 989 |
| 10001100 | GTAGTCACTGACTGAC | 734 | TGACTCACTGACTCAG | 990 |
| 10001101 | GTAGTCACTGAGTGAC | 735 | TGACTCACTGAGTCAG | 991 |
| 10001110 | GTAGTCACTGTCTGAC | 736 | TGACTCACTGTCTCAG | 992 |
| 10001111 | GTAGTCACTGTGTGAC | 737 | TGACTCACTGTGTCAG | 993 |
| 10010000 | GTAGTCAGACACTGAC | 738 | TGACTCAGACACTCAG | 994 |
| 10010001 | GTAGTCAGACAGTGAC | 739 | TGACTCAGACAGTCAG | 995 |
| 10010010 | GTAGTCAGACTCTGAC | 740 | TGACTCAGACTCTCAG | 996 |
| 10010011 | GTAGTCAGACTGTGAC | 741 | TGACTCAGACTGTCAG | 997 |
| 10010100 | GTAGTCAGAGACTGAC | 742 | TGACTCAGAGACTCAG | 998 |
| 10010101 | GTAGTCAGAGAGTGAC | 743 | TGACTCAGAGAGTCAG | 999 |
| 10010110 | GTAGTCAGAGTCTGAC | 744 | TGACTCAGAGTCTCAG | 1000 |
| 10010111 | GTAGTCAGAGTGTGAC | 745 | TGACTCAGAGTGTCAG | 1001 |
| 10011000 | GTAGTCAGTCACTGAC | 746 | TGACTCAGTCACTCAG | 1002 |
| 10011001 | GTAGTCAGTCAGTGAC | 747 | TGACTCAGTCAGTCAG | 1003 |
| 10011010 | GTAGTCAGTCTCTGAC | 748 | TGACTCAGTCTCTCAG | 1004 |
| 10011011 | GTAGTCAGTCTGTGAC | 749 | TGACTCAGTCTGTCAG | 1005 |
| 10011100 | GTAGTCAGTGACTGAC | 750 | TGACTCAGTGACTCAG | 1006 |
| 10011101 | GTAGTCAGTGAGTGAC | 751 | TGACTCAGTGAGTCAG | 1007 |
| 10011110 | GTAGTCAGTGTCTGAC | 752 | TGACTCAGTGTCTCAG | 1008 |
| 10011111 | GTAGTCAGTGTGTGAC | 753 | TGACTCAGTGTGTCAG | 1009 |
| 10100000 | GTAGTCTCACACTGAC | 754 | TGACTCTCACACTCAG | 1010 |
| 10100001 | GTAGTCTCACAGTGAC | 755 | TGACTCTCACAGTCAG | 1011 |
| 10100010 | GTAGTCTCACTCTGAC | 756 | TGACTCTCACTCTCAG | 1012 |
| 10100011 | GTAGTCTCACTGTGAC | 757 | TGACTCTCACTGTCAG | 1013 |
| 10100100 | GTAGTCTCAGACTGAC | 758 | TGACTCTCAGACTCAG | 1014 |
| 10100101 | GTAGTCTCAGAGTGAC | 759 | TGACTCTCAGAGTCAG | 1015 |
| 10100110 | GTAGTCTCAGTCTGAC | 760 | TGACTCTCAGTCTCAG | 1016 |
| 10100111 | GTAGTCTCAGTGTGAC | 761 | TGACTCTCAGTGTCAG | 1017 |
| 10101000 | GTAGTCTCTCACTGAC | 762 | TGACTCTCTCACTCAG | 1018 |
| 10101001 | GTAGTCTCTCAGTGAC | 763 | TGACTCTCTCAGTCAG | 1019 |
| 10101010 | GTAGTCTCTCTCTGAC | 764 | TGACTCTCTCTCTCAG | 1020 |
| 10101011 | GTAGTCTCTCTGTGAC | 765 | TGACTCTCTCTGTCAG | 1021 |
| 10101100 | GTAGTCTCTGACTGAC | 766 | TGACTCTCTGACTCAG | 1022 |
| 10101101 | GTAGTCTCTGAGTGAC | 767 | TGACTCTCTGAGTCAG | 1023 |
| 10101110 | GTAGTCTCTGTCTGAC | 768 | TGACTCTCTGTCTCAG | 1024 |
| 10101111 | GTAGTCTCTGTGTGAC | 769 | TGACTCTCTGTGTCAG | 1025 |
| 10110000 | GTAGTCTGACACTGAC | 770 | TGACTCTGACACTCAG | 1026 |
| 10110001 | GTAGTCTGACAGTGAC | 771 | TGACTCTGACAGTCAG | 1027 |
| 10110010 | GTAGTCTGACTCTGAC | 772 | TGACTCTGACTCTCAG | 1028 |
| 10110011 | GTAGTCTGACTGTGAC | 773 | TGACTCTGACTGTCAG | 1029 |
| 10110100 | GTAGTCTGAGACTGAC | 774 | TGACTCTGAGACTCAG | 1030 |
| 10110101 | GTAGTCTGAGAGTGAC | 775 | TGACTCTGAGAGTCAG | 1031 |
| 10110110 | GTAGTCTGAGTCTGAC | 776 | TGACTCTGAGTCTCAG | 1032 |
| 10110111 | GTAGTCTGAGTGTGAC | 777 | TGACTCTGAGTGTCAG | 1033 |
| 10111000 | GTAGTCTGTCACTGAC | 778 | TGACTCTGTCACTCAG | 1034 |
| 10111001 | GTAGTCTGTCAGTGAC | 779 | TGACTCTGTCAGTCAG | 1035 |
| 10111010 | GTAGTCTGTCTCTGAC | 780 | TGACTCTGTCTCTCAG | 1036 |
| 10111011 | GTAGTCTGTCTGTGAC | 781 | TGACTCTGTCTGTCAG | 1037 |
| 10111100 | GTAGTCTGTGACTGAC | 782 | TGACTCTGTGACTCAG | 1038 |
| 10111101 | GTAGTCTGTGAGTGAC | 783 | TGACTCTGTGAGTCAG | 1039 |
| 10111110 | GTAGTCTGTGTCTGAC | 784 | TGACTCTGTGTCTCAG | 1040 |
| 10111111 | GTAGTCTGTGTGTGAC | 785 | TGACTCTGTGTGTCAG | 1041 |
| 11000000 | GTAGTGACACACTGAC | 786 | TGACTGACACACTCAG | 1042 |
| 11000001 | GTAGTGACACAGTGAC | 787 | TGACTGACACAGTCAG | 1043 |
| 11000010 | GTAGTGACACTCTGAC | 788 | TGACTGACACTCTCAG | 1044 |
| 11000011 | GTAGTGACACTGTGAC | 789 | TGACTGACACTGTCAG | 1045 |
| 11000100 | GTAGTGACAGACTGAC | 790 | TGACTGACAGACTCAG | 1046 |
| 11000101 | GTAGTGACAGAGTGAC | 791 | TGACTGACAGAGTCAG | 1047 |
| 11000110 | GTAGTGACAGTCTGAC | 792 | TGACTGACAGTCTCAG | 1048 |
| 11000111 | GTAGTGACAGTGTGAC | 793 | TGACTGACAGTGTCAG | 1049 |
| 11001000 | GTAGTGACTCACTGAC | 794 | TGACTGACTCACTCAG | 1050 |
| 11001001 | GTAGTGACTCAGTGAC | 795 | TGACTGACTCAGTCAG | 1051 |
| 11001010 | GTAGTGACTCTCTGAC | 796 | TGACTGACTCTCTCAG | 1052 |
| 11001011 | GTAGTGACTCTGTGAC | 797 | TGACTGACTCTGTCAG | 1053 |
| 11001100 | GTAGTGACTGACTGAC | 798 | TGACTGACTGACTCAG | 1054 |
| 11001101 | GTAGTGACTGAGTGAC | 799 | TGACTGACTGAGTCAG | 1055 |
| 11001110 | GTAGTGACTGTCTGAC | 800 | TGACTGACTGTCTCAG | 1056 |
| 11001111 | GTAGTGACTGTGTGAC | 801 | TGACTGACTGTGTCAG | 1057 |
| 11010000 | GTAGTGAGACACTGAC | 802 | TGACTGAGACACTCAG | 1058 |
| 11010001 | GTAGTGAGACAGTGAC | 803 | TGACTGAGACAGTCAG | 1059 |
| 11010010 | GTAGTGAGACTCTGAC | 804 | TGACTGAGACTCTCAG | 1060 |
| 11010011 | GTAGTGAGACTGTGAC | 805 | TGACTGAGACTGTCAG | 1061 |
| 11010100 | GTAGTGAGAGACTGAC | 806 | TGACTGAGAGACTCAG | 1062 |
| 11010101 | GTAGTGAGAGAGTGAC | 807 | TGACTGAGAGAGTCAG | 1063 |
| 11010110 | GTAGTGAGAGTCTGAC | 808 | TGACTGAGAGTCTCAG | 1064 |
| 11010111 | GTAGTGAGAGTGTGAC | 809 | TGACTGAGAGTGTCAG | 1065 |
| 11011000 | GTAGTGAGTCACTGAC | 810 | TGACTGAGTCACTCAG | 1066 |
| 11011001 | GTAGTGAGTCAGTGAC | 811 | TGACTGAGTCAGTCAG | 1067 |
| 11011010 | GTAGTGAGTCTCTGAC | 812 | TGACTGAGTCTCTCAG | 1068 |
| 11011011 | GTAGTGAGTCTGTGAC | 813 | TGACTGAGTCTGTCAG | 1069 |
| 11011100 | GTAGTGAGTGACTGAC | 814 | TGACTGAGTGACTCAG | 1070 |
| 11011101 | GTAGTGAGTGAGTGAC | 815 | TGACTGAGTGAGTCAG | 1071 |
| 11011110 | GTAGTGAGTGTCTGAC | 816 | TGACTGAGTGTCTCAG | 1072 |
| 11011111 | GTAGTGAGTGTGTGAC | 817 | TGACTGAGTGTGTCAG | 1073 |
| 11100000 | GTAGTGTCACACTGAC | 818 | TGACTGTCACACTCAG | 1074 |
| 11100001 | GTAGTGTCACAGTGAC | 819 | TGACTGTCACAGTCAG | 1075 |
| 11100010 | GTAGTGTCACTCTGAC | 820 | TGACTGTCACTCTCAG | 1076 |
| 11100011 | GTAGTGTCACTGTGAC | 821 | TGACTGTCACTGTCAG | 1077 |
| 11100100 | GTAGTGTCAGACTGAC | 822 | TGACTGTCAGACTCAG | 1078 |
| 11100101 | GTAGTGTCAGAGTGAC | 823 | TGACTGTCAGAGTCAG | 1079 |
| 11100110 | GTAGTGTCAGTCTGAC | 824 | TGACTGTCAGTCTCAG | 1080 |
| 11100111 | GTAGTGTCAGTGTGAC | 825 | TGACTGTCAGTGTCAG | 1081 |
| 11101000 | GTAGTGTCTCACTGAC | 826 | TGACTGTCTCACTCAG | 1082 |
| 11101001 | GTAGTGTCTCAGTGAC | 827 | TGACTGTCTCAGTCAG | 1083 |
| 11101010 | GTAGTGTCTCTCTGAC | 828 | TGACTGTCTCTCTCAG | 1084 |
| 11101011 | GTAGTGTCTCTGTGAC | 829 | TGACTGTCTCTGTCAG | 1085 |
| 11101100 | GTAGTGTCTGACTGAC | 830 | TGACTGTCTGACTCAG | 1086 |
| 11101101 | GTAGTGTCTGAGTGAC | 831 | TGACTGTCTGAGTCAG | 1087 |
| 11101110 | GTAGTGTCTGTCTGAC | 832 | TGACTGTCTGTCTCAG | 1088 |
| 11101111 | GTAGTGTCTGTGTGAC | 833 | TGACTGTCTGTGTCAG | 1089 |
| 11110000 | GTAGTGTGACACTGAC | 834 | TGACTGTGACACTCAG | 1090 |
| 11110001 | GTAGTGTGACAGTGAC | 835 | TGACTGTGACAGTCAG | 1091 |
| 11110010 | GTAGTGTGACTCTGAC | 836 | TGACTGTGACTCTCAG | 1092 |
| 11110011 | GTAGTGTGACTGTGAC | 837 | TGACTGTGACTGTCAG | 1093 |
| 11110100 | GTAGTGTGAGACTGAC | 838 | TGACTGTGAGACTCAG | 1094 |
| 11110101 | GTAGTGTGAGAGTGAC | 839 | TGACTGTGAGAGTCAG | 1095 |
| 11110110 | GTAGTGTGAGTCTGAC | 840 | TGACTGTGAGTCTCAG | 1096 |
| 11110111 | GTAGTGTGAGTGTGAC | 841 | TGACTGTGAGTGTCAG | 1097 |
| 11111000 | GTAGTGTGTCACTGAC | 842 | TGACTGTGTCACTCAG | 1098 |
| 11111001 | GTAGTGTGTCAGTGAC | 843 | TGACTGTGTCAGTCAG | 1099 |
| 11111010 | GTAGTGTGTCTCTGAC | 844 | TGACTGTGTCTCTCAG | 1100 |
| 11111011 | GTAGTGTGTCTGTGAC | 845 | TGACTGTGTCTGTCAG | 1101 |
| 11111100 | GTAGTGTGTGACTGAC | 846 | TGACTGTGTGACTCAG | 1102 |
| 11111101 | GTAGTGTGTGAGTGAC | 847 | TGACTGTGTGAGTCAG | 1103 |
| 11111110 | GTAGTGTGTGTCTGAC | 848 | TGACTGTGTGTCTCAG | 1104 |
| 11111111 | GTAGTGTGTGTGTGAC | 849 | TGACTGTGTGTGTCAG | 1105 |
| EOF_B | | | TGACCAGTCTGTTCAG | 1106 |

The presence of the BsaI cleavage site in the library plasmids allows to capture the 1146 required biooctets surrounded by fusion sites, alternating between library A and library B. The plasmids containing the required biooctets from each library are digested by the restriction enzyme BsaI, thus releasing the 1146 biooctets surrounded by their fusion sites. After capturing the x=1146 biooctets surrounded by their cleavage sites they are assembled together in a fixed order in three steps.

At step 1, blocks containing 2 biooctets (BioblockX2) are assembled from the 1146 biooctets surrounded by their fusion sites in double-stranded replicative plasmids. Each plasmid contains two internal BsaI cleavage sites in opposite orientation allowing to release, after BsaI cleavage, the fusion sites GTAG and TCAG upstream and downstream of the BioblockX2 respectively. The fusion sites surrounding each biooctet in libraries A and B allow to assemble biooctets from library A in first position and biooctets of library B in second position. The BioblockX2 are assembled in a set of 32 double-stranded replicative plasmids containing regions surrounding BioblockX2 and comprising a cleavage site for the type IIs restriction enzyme BsmBI (FIG. 3). The variable region of the BsmBI cleavage site is defined for each of the 32 plasmids and define ordered positions for assembly of groups of 32 BioblockX2 at step 2 of the assembly process, thanks to a set of 33 fusion sites (Table 5).

TABLE 5
Fusion sites surrounding BioblockX2 in the 32
recipient plasmids

FS1_0 = AATA
FS1_1 = TCAA
FS1_2 = CTTC
FS1_3 = AGTA
FS1_4 = ACTG
FS1_5 = CACA
FS1_6 = CCAG
FS1 7 = CAAA
FS1_8 = GACC
FS1 9 = ACTC
FS1_10 = CCAC
FS1 11 = GAAC
FS1_12 = GCAC
FS1_13 = CGGC
FS1_14 = CGTA
FS1_15 = GTAA
FS1_16 = CAAC
FS1_17 = GCTA
FS1_18 = CCGA
FS1_19 = ACGA
FS1_20 = AGAA
FS1_21 = TAAA
FS1_22 = AGCG
FS1_23 = ACCT
FS1_24 = AACA
FS1_25 = GGCA
FS1_26 = ACGC
FS1_27 = AATC
FS1_28 = CGAG
FS1_29 = TCCA
FS1_30 = CCTA
FS1_31 = CTAA
FS1_32 = GGGA

A total of 573 plasmids are assembled at step 1. The 36-nucleotide sequences of the 573 BioblockX2 and their surrounding fusion sites correspond to SEQ ID NO: 1 to SEQ ID NO: 573. The first and last group of 4 nucleotides correspond to the fusion sites flanking each BioblockX2. The groups of 4 nucleotides at positions 5-8, 17-20 and 29-32 correspond to the fusion sites from the bioblocks derived from libraries A and B.

As an example, BioblockX2_0 has the following sequence: AATAGTAGACACACACTGACACACACACTCAGTCAA (SEQ ID NO: 1). The fusion sites from the bioblocks derived from libraries A and B are bolded, the fusion sites flanking each BioblockX2 (FS1_X) are italicized.

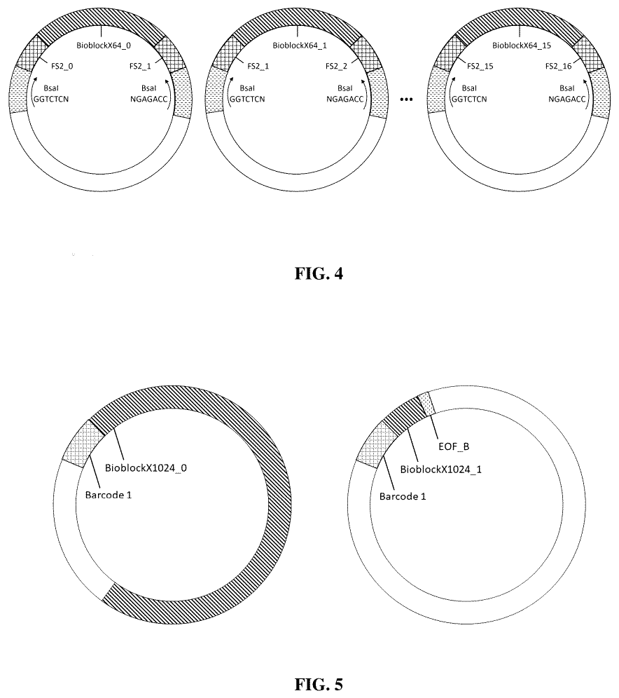

At step 2, the x=573 BioblockX2 and their surrounding fusion sites are captured by digestion with the BsmBI restriction enzyme and assembled into BioblockX64 comprising 32 BioblockX2 in double-stranded replicative plasmids. Each plasmid contains two internal BsmBI cleavage sites in opposite orientation allowing to release, after BsmBI cleavage, fusion site FS1_0 and fusion site FS1_32 upstream and downstream of the BioblockX64 respectively. The BioblockX2 are assembled in the correct order thanks to the 33 fusion sites in a set of 16 double-stranded replicative plasmids containing regions surrounding BioblockX64 and comprising a cleavage site for the type IIs restriction enzyme BsaI (FIG. 4). The variable region of the BsaI cleavage site is different for each of the 16 plasmids and define ordered positions for assembly of groups of 16 bioblockX64 at step 3 of the assembly process, thanks to a set of 17 fusion sites (Table 6).

TABLE 6
Fusion sites surrounding BioblockX64 in the 16
recipient plasmids

FS2_0 = AATA
FS2_1 = AAGG
FS2_2 = AAAC
FS2_3 = TAAA
FS2_4 = ACGA
FS2_5 = ACTG
FS2_6 = AGCG
FS2_7 = GCTA
FS2_8 = GGCA
FS2_9 = ACCT
FS2_10 = CGTA
FS2_11 = AACA
FS2_12 = CTAC
FS2_13 = GAGA
FS2_14 = CCAG
FS2_15 = AGAA
FS2_16 = GCAC

A total of 18 plasmids are assembled at step 2, 17 of them contain 32 BioblockX2, while the last one contains 29 BioblockX2. The sequences of the 18 BioblockX64 and their surrounding fusion sites correspond to SEQ ID NO: 574 to SEQ ID NO: 591.

At step 3, the x=18 BioblockX64 and their surrounding fusion sites are captured by digestion with the BsaI restriction enzyme and assembled into BioblockX1024 comprising 16 BioblockX64 in double-stranded replicative plasmids. Each plasmid contains two internal BsaI cleavage sites in opposite orientation allowing to release, after BsaI cleavage, fusion site FS2_0 and fusion site FS2_16 (Table 6) upstream and downstream of the BioblockX1024 respectively. The bioblockX64 are assembled in the correct order thanks to the 17 fusion sites (Table 6).

At step 3, two plasmids corresponding to track 0 and track 1 are assembled (FIG. 5). The sequences of the two BioblockX1024 correspond to SEQ ID NO: 592 and SEQ ID NO: 593. Track 0 comprises 1024 biooctets (four barcoding biooctets and the first 1020 biooctets of file B). Track 1 comprises 122 biooctets (four barcoding biooctets, the last 117 biooctets of file B and the special EOF_B biooctet).

1-15. (canceled)

16. A nucleic acid-based data storage method for storing information comprising:
a) recovering data in the form of a digital sequence formed of a plurality of bits, each bit having the value 0 or 1,
b) subdividing the digital sequence into n digital subsequences, each comprising m bits, m being comprised between 2 and 16,
c) converting each of the n digital subsequences into a bioblock, a bioblock consisting of a sequence of m nucleotides,
wherein the digital subsequence consists in m bits assigned to positions 0 to m−1, and
wherein the conversion of a digital subsequence into a bioblock consists in:
converting bits at even positions to a first nucleotide N1 if said bits has the value 0, and to a second distinct nucleotide N2 if said bits has the value 1 and
converting bits at odd positions to a third nucleotide N3 if said bits has the value 0, and to a fourth distinct nucleotide N4 if said bits has the value 1,
wherein N1, N2, N3 and N4 are distinct nucleotides
d) constructing a plurality of x components, each individual component of the plurality of x components comprising at least one bioblock, and the x components together comprising n bioblocks
e) assembling together in a fixed order, in one or more steps, the plurality of x components.

17. The nucleic acid-based data storage method according to claim 16, wherein the nucleotides are selected from the group of natural nucleotides consisting of adenine, guanine, cytosine, uracil and thymine or from non-natural nucleotides.

18. The nucleic acid-based data storage method according to claim 16, wherein the x components are x DNA molecules, preferably x double-stranded DNA molecules.

19. The nucleic acid-based data storage method according to claim 16, wherein at step (d) the construction of a plurality of x components, each comprising at least one bioblock, comprises the steps of:
selectively capturing x data storage nucleic acid molecules from at least one library of data storage nucleic acid molecules, wherein each data storage nucleic acid molecule comprises at least one bioblock surrounded by regions comprising cleavage sites,
cleaving each of the x data storage nucleic acid molecules, thereby releasing the at least one bioblock.

20. The nucleic acid-based data storage method according to claim 19 wherein at step (d) the construction of a plurality of x components, each comprising at least one bioblock, comprises the steps of
selectively capturing n data storage nucleic acid molecules from at least two libraries of data storage nucleic acid molecules, wherein each data storage nucleic acid molecule of each library comprises one bioblock surrounded by regions comprising cleavage sites, and wherein each library comprises all possible bioblocks of m nucleotides,
cleaving each of the n data storage nucleic acid molecules, thereby releasing the n bioblocks.

21. The nucleic acid-based data storage method according to claim 19, wherein the regions comprising cleavage sites comprises from 2 to 25 nucleotides.

22. The nucleic acid-based data storage method according to claim 19, wherein each of the region surrounding each bioblock comprises a site for a restriction enzyme, and step (d) comprises a step of digesting each of the x data storage nucleic acid molecules with one or two restriction enzymes.

23. The nucleic acid-based data storage method according to claim 16, wherein step (e) comprises one or several assembling steps using overlap-extension polymerase chain reaction (PCR), polymerase cycling assembly, sticky end ligation, biobricks assembly, golden gate assembly, Gibson assembly, recombinase assembly, ligase cycling reaction, template directed ligation, in vivo assembly or any other DNA assembly protocol.

24. A data storage nucleic acid molecule comprising at least one bioblock, a bioblock consisting of a nucleic acid sequence consisting of m nucleotides assigned to positions 0 to m−1, wherein
a bioblock is formed of at least 2 and at most 4 distinct nucleotides
nucleotides at even positions may be selected from a first and a second nucleotide, and nucleotides at odd positions may be selected from a third and a fourth nucleotide, said first, second, third and fourth nucleotides being distinct.

25. The data storage nucleic acid molecule according to claim 24, being a double-stranded molecule, preferably a DNA molecule.

26. The data storage nucleic acid molecule according to claim 24, being a plasmid, a cosmid, a fosmid, a prokaryotic chromosome or a eukaryotic chromosome.

27. The data storage nucleic acid molecule according to claim 24, wherein each of the bioblock is surrounded by regions comprising cleavage sites, preferably by two sites for one restriction enzyme.

28. The data storage nucleic acid molecule according to claim 24 being replicative.

29. A library comprising a plurality of data storage nucleic acid molecules according to claim 24, wherein each of the data storage nucleic acid molecule of the library contains one bioblock, wherein each data storage nucleic acid molecule of the library comprises the same surrounding regions comprising cleavage sites and wherein the library contains all possible bioblocks of m nucleotides.

30. A nucleic acid-based data storage system comprising at least two libraries according to claim 29.


SEQUENCE LISTING (1107 sequences)

SEQ ID NO: 1 - DNA, 36 bp, Artificial Sequence
  >BioblockX2_0
aatagtagacacacactgacacacacactcagtcaa

SEQ ID NO: 2 - DNA, 36 bp, Artificial Sequence
  >BioblockX2_1
tcaagtagacacacactgacacacacactcagcttc

SEQ ID NO: 3 - DNA, 36 bp, Artificial Sequence
  >BioblockX2_2
cttcgtagactgagtgtgacagtgtctctcagagta

SEQ ID NO: 4 - DNA, 36 bp, Artificial Sequence
  >BioblockX2_3
agtagtagtctgtgactgactctctgtgtcagactg

SEQ ID NO: 5 - DNA, 36 bp, Artificial Sequence
  >BioblockX2_4
actggtagactcagtgtgacacagtgactcagcaca

SEQ ID NO: 6 - DNA, 36 bp, Artificial Sequence
  >BioblockX2_5
cacagtagacacacactgacacacagactcagccag

SEQ ID NO: 7 - DNA, 36 bp, Artificial Sequence
  >BioblockX2_6
ccaggtagtgagagactgactgagtgtgtcagcaaa

SEQ ID NO: 8 - DNA, 36 bp, Artificial Sequence
  >BioblockX2_7
caaagtagtgtgtcagtgactctgtgactcaggacc

SEQ ID NO: 9 - DNA, 36 bp, Artificial Sequence
  >BioblockX2_8
gaccgtagtgtctgtgtgacacacactgtcagactc

SEQ ID NO: 10 - DNA, 36 bp, Artificial Sequence
  >BioblockX2_9
actcgtagacacacactgacacacacactcagccac

SEQ ID NO: 11 - DNA, 36 bp, Artificial Sequence
  >BioblockX2_10
ccacgtagacacacactgacacacacactcaggaac

SEQ ID NO: 12 - DNA, 36 bp, Artificial Sequence
  >BioblockX2_11
gaacgtagacacacactgacacacacactcaggcac

SEQ ID NO: 13 - DNA, 36 bp, Artificial Sequence
  >BioblockX2_12
gcacgtagagtcactctgacacacacactcagcggc

SEQ ID NO: 14 - DNA, 36 bp, Artificial Sequence
  >BioblockX2_13
cggcgtagacacacactgacacacacactcagcgta

SEQ ID NO: 15 - DNA, 36 bp, Artificial Sequence
  >BioblockX2_14
cgtagtagacacacactgacacacacactcaggtaa

SEQ ID NO: 16 - DNA, 36 bp, Artificial Sequence
  >BioblockX2_15
gtaagtagacacacactgacacacacactcagcaac

SEQ ID NO: 17 - DNA, 36 bp, Artificial Sequence
  >BioblockX2_16
caacgtagtgagtcactgacagactgtctcaggcta

SEQ ID NO: 18 - DNA, 36 bp, Artificial Sequence
  >BioblockX2_17
gctagtagtcagagactgactcagtgtgtcagccga

SEQ ID NO: 19 - DNA, 36 bp, Artificial Sequence
  >BioblockX2_18
ccgagtagtgtcacactgacacactcagtcagacga

SEQ ID NO: 20 - DNA, 36 bp, Artificial Sequence
  >BioblockX2_19
acgagtagactgagagtgacacacactgtcagagaa

SEQ ID NO: 21 - DNA, 36 bp, Artificial Sequence
  >BioblockX2_20
agaagtagtgtcagtgtgacagagtgagtcagtaaa

SEQ ID NO: 22 - DNA, 36 bp, Artificial Sequence
  >BioblockX2_21
taaagtagacacacactgacactcagtctcagagcg

SEQ ID NO: 23 - DNA, 36 bp, Artificial Sequence
  >BioblockX2_22
agcggtagacagtctctgacagactcactcagacct

SEQ ID NO: 24 - DNA, 36 bp, Artificial Sequence
  >BioblockX2_23
acctgtagagacagtctgacagtgacagtcagaaca

SEQ ID NO: 25 - DNA, 36 bp, Artificial Sequence
  >BioblockX2_24
aacagtagtgtctctctgacactcacagtcagggca

SEQ ID NO: 26 - DNA, 36 bp, Artificial Sequence
  >BioblockX2_25
ggcagtagactgtgtctgacagactcagtcagacgc

SEQ ID NO: 27 - DNA, 36 bp, Artificial Sequence
  >BioblockX2_26
acgcgtagtctctgtgtgactgtctgtgtcagaatc

SEQ ID NO: 28 - DNA, 36 bp, Artificial Sequence
  >BioblockX2_27
aatcgtagtgtcacagtgactctgtgtctcagcgag

SEQ ID NO: 29 - DNA, 36 bp, Artificial Sequence
  >BioblockX2_28
cgaggtagagtcacactgactgagacagtcagtcca

SEQ ID NO: 30 - DNA, 36 bp, Artificial Sequence
  >BioblockX2_29
tccagtagacagactctgacagtgactgtcagccta

SEQ ID NO: 31 - DNA, 36 bp, Artificial Sequence
  >BioblockX2_30
cctagtagagtgtgactgactcacagtctcagctaa

SEQ ID NO: 32 - DNA, 36 bp, Artificial Sequence
  >BioblockX2_31
ctaagtagacacagactgacagacacactcagggga

SEQ ID NO: 33 - DNA, 36 bp, Artificial Sequence
  >BioblockX2_32
aatagtagacacactctgactgtctgtgtcagtcaa

SEQ ID NO: 34 - DNA, 36 bp, Artificial Sequence
  >BioblockX2_33
tcaagtagtgagagtgtgactctcactgtcagcttc

SEQ ID NO: 35 - DNA, 36 bp, Artificial Sequence
  >BioblockX2_34
cttcgtagtcagtcactgactcactctctcagagta

SEQ ID NO: 36 - DNA, 36 bp, Artificial Sequence
  >BioblockX2_35
agtagtagacacactgtgactgagtgtctcagactg

SEQ ID NO: 37 - DNA, 36 bp, Artificial Sequence
  >BioblockX2_36
actggtagtgtgagtctgacacactgagtcagcaca

SEQ ID NO: 38 - DNA, 36 bp, Artificial Sequence
  >BioblockX2_37
cacagtagagagtgactgactctcagagtcagccag

SEQ ID NO: 39 - DNA, 36 bp, Artificial Sequence
  >BioblockX2_38
ccaggtagtcagtcagtgactgagacactcagcaaa

SEQ ID NO: 40 - DNA, 36 bp, Artificial Sequence
  >BioblockX2_39
caaagtagacagtgagtgacagtgactgtcaggacc

SEQ ID NO: 41 - DNA, 36 bp, Artificial Sequence
  >BioblockX2_40
gaccgtagactgtgtgtgacacagacactcagactc

SEQ ID NO: 42 - DNA, 36 bp, Artificial Sequence
  >BioblockX2_41
actcgtagtgtgtgactgacactcacactcagccac

SEQ ID NO: 43 - DNA, 36 bp, Artificial Sequence
  >BioblockX2_42
ccacgtagtctgactgtgacagtcagtctcaggaac

SEQ ID NO: 44 - DNA, 36 bp, Artificial Sequence
  >BioblockX2_43
gaacgtagacacagactgacactctctctcaggcac

SEQ ID NO: 45 - DNA, 36 bp, Artificial Sequence
  >BioblockX2_44
gcacgtagagacacagtgactctcacagtcagcggc

SEQ ID NO: 46 - DNA, 36 bp, Artificial Sequence
  >BioblockX2_45
cggcgtagtgtctgactgacacacagactcagcgta

SEQ ID NO: 47 - DNA, 36 bp, Artificial Sequence
  >BioblockX2_46
cgtagtagacacagtgtgactctctctgtcaggtaa

SEQ ID NO: 48 - DNA, 36 bp, Artificial Sequence
  >BioblockX2_47
gtaagtagtcagtctgtgactcacagtctcagcaac

SEQ ID NO: 49 - DNA, 36 bp, Artificial Sequence
  >BioblockX2_48
caacgtagagtcagtctgactgtctgactcaggcta

SEQ ID NO: 50 - DNA, 36 bp, Artificial Sequence
  >BioblockX2_49
gctagtagtctcactgtgactctctctgtcagccga

SEQ ID NO: 51 - DNA, 36 bp, Artificial Sequence
  >BioblockX2_50
ccgagtagactgagagtgactctcacagtcagacga

SEQ ID NO: 52 - DNA, 36 bp, Artificial Sequence
  >BioblockX2_51
acgagtagtgagtctctgacacagtctctcagagaa

SEQ ID NO: 53 - DNA, 36 bp, Artificial Sequence
  >BioblockX2_52
agaagtagacacagactgacacactgtgtcagtaaa

SEQ ID NO: 54 - DNA, 36 bp, Artificial Sequence
  >BioblockX2_53
taaagtagactctctgtgacagactctgtcagagcg

SEQ ID NO: 55 - DNA, 36 bp, Artificial Sequence
  >BioblockX2_54
agcggtagtcagtcactgacactgagagtcagacct

SEQ ID NO: 56 - DNA, 36 bp, Artificial Sequence
  >BioblockX2_55
acctgtagtgacagactgactgagacagtcagaaca

SEQ ID NO: 57 - DNA, 36 bp, Artificial Sequence
  >BioblockX2_56
aacagtagtgtcagtctgacagtcactctcagggca

SEQ ID NO: 58 - DNA, 36 bp, Artificial Sequence
  >BioblockX2_57
ggcagtagacagacagtgactgtcagtctcagacgc

SEQ ID NO: 59 - DNA, 36 bp, Artificial Sequence
  >BioblockX2_58
acgcgtagtctctctgtgactgtctcagtcagaatc

SEQ ID NO: 60 - DNA, 36 bp, Artificial Sequence
  >BioblockX2_59
aatcgtagagtgtgagtgacagagtctctcagcgag

SEQ ID NO: 61 - DNA, 36 bp, Artificial Sequence
  >BioblockX2_60
cgaggtagactctgtgtgactctgtctctcagtcca

SEQ ID NO: 62 - DNA, 36 bp, Artificial Sequence
  >BioblockX2_61
tccagtagactgagagtgactcagtgtctcagccta

SEQ ID NO: 63 - DNA, 36 bp, Artificial Sequence
  >BioblockX2_62
cctagtagacactgagtgactctcagtctcagctaa

SEQ ID NO: 64 - DNA, 36 bp, Artificial Sequence
  >BioblockX2_63
ctaagtagactctgagtgacagtgagtgtcagggga

SEQ ID NO: 65 - DNA, 36 bp, Artificial Sequence
  >BioblockX2_64
aatagtagacagacagtgacacacactgtcagtcaa

SEQ ID NO: 66 - DNA, 36 bp, Artificial Sequence
  >BioblockX2_65
tcaagtagacagagactgactgtcactgtcagcttc

SEQ ID NO: 67 - DNA, 36 bp, Artificial Sequence
  >BioblockX2_66
cttcgtagtgactctgtgacagagagtctcagagta

SEQ ID NO: 68 - DNA, 36 bp, Artificial Sequence
  >BioblockX2_67
agtagtagtcactctgtgacacagtgtgtcagactg

SEQ ID NO: 69 - DNA, 36 bp, Artificial Sequence
  >BioblockX2_68
actggtagtgtcagtgtgacactgtcagtcagcaca

SEQ ID NO: 70 - DNA, 36 bp, Artificial Sequence
  >BioblockX2_69
cacagtagtgtgtcagtgactctgactctcagccag

SEQ ID NO: 71 - DNA, 36 bp, Artificial Sequence
  >BioblockX2_70
ccaggtagactctctctgacagtgtgagtcagcaaa

SEQ ID NO: 72 - DNA, 36 bp, Artificial Sequence
  >BioblockX2_71
caaagtagactcagagtgacagacacactcaggacc

SEQ ID NO: 73 - DNA, 36 bp, Artificial Sequence
  >BioblockX2_72
gaccgtagtgacagtgtgacactgtcactcagactc

SEQ ID NO: 74 - DNA, 36 bp, Artificial Sequence
  >BioblockX2_73
actcgtagactctgtgtgacagtgagagtcagccac

SEQ ID NO: 75 - DNA, 36 bp, Artificial Sequence
  >BioblockX2_74
ccacgtagtcagtgagtgactcagtgtgtcaggaac

SEQ ID NO: 76 - DNA, 36 bp, Artificial Sequence
  >BioblockX2_75
gaacgtagagtcacagtgacacagtgagtcaggcac

SEQ ID NO: 77 - DNA, 36 bp, Artificial Sequence
  >BioblockX2_76
gcacgtagagtgtcactgactctgtcagtcagcggc

SEQ ID NO: 78 - DNA, 36 bp, Artificial Sequence
  >BioblockX2_77
cggcgtagtcagtctgtgactctctgtctcagcgta

SEQ ID NO: 79 - DNA, 36 bp, Artificial Sequence
  >BioblockX2_78
cgtagtagactgagtgtgacagacacactcaggtaa

SEQ ID NO: 80 - DNA, 36 bp, Artificial Sequence
  >BioblockX2_79
gtaagtagtgtgagagtgactcacagtctcagcaac

SEQ ID NO: 81 - DNA, 36 bp, Artificial Sequence
  >BioblockX2_80
caacgtagactgtgagtgactgacactgtcaggcta

SEQ ID NO: 82 - DNA, 36 bp, Artificial Sequence
  >BioblockX2_81
gctagtagagagtcagtgactcagagtctcagccga

SEQ ID NO: 83 - DNA, 36 bp, Artificial Sequence
  >BioblockX2_82
ccgagtagtgtcacactgactgactctctcagacga

SEQ ID NO: 84 - DNA, 36 bp, Artificial Sequence
  >BioblockX2_83
acgagtagagtctctctgactcagagtgtcagagaa

SEQ ID NO: 85 - DNA, 36 bp, Artificial Sequence
  >BioblockX2_84
agaagtagactgagtgtgactctcactctcagtaaa

SEQ ID NO: 86 - DNA, 36 bp, Artificial Sequence
  >BioblockX2_85
taaagtagtgtcagagtgacagtgacagtcagagcg

SEQ ID NO: 87 - DNA, 36 bp, Artificial Sequence
  >BioblockX2_86
agcggtagtgtgactgtgacagtctcagtcagacct

SEQ ID NO: 88 - DNA, 36 bp, Artificial Sequence
  >BioblockX2_87
acctgtagtgtgtctctgactgtcagactcagaaca

SEQ ID NO: 89 - DNA, 36 bp, Artificial Sequence
  >BioblockX2_88
aacagtagactctctgtgactcagacagtcagggca

SEQ ID NO: 90 - DNA, 36 bp, Artificial Sequence
  >BioblockX2_89
ggcagtagtgtgagtgtgacactcagtgtcagacgc

SEQ ID NO: 91 - DNA, 36 bp, Artificial Sequence
  >BioblockX2_90
acgcgtagtctgtgagtgactgagacagtcagaatc

SEQ ID NO: 92 - DNA, 36 bp, Artificial Sequence
  >BioblockX2_91
aatcgtagtgagagtctgacagagactgtcagcgag

SEQ ID NO: 93 - DNA, 36 bp, Artificial Sequence
  >BioblockX2_92
cgaggtagagagtgtgtgactctcagactcagtcca

SEQ ID NO: 94 - DNA, 36 bp, Artificial Sequence
  >BioblockX2_93
tccagtagagagagagtgacagagagtgtcagccta

SEQ ID NO: 95 - DNA, 36 bp, Artificial Sequence
  >BioblockX2_94
cctagtagacacagtctgacagagtctctcagctaa

SEQ ID NO: 96 - DNA, 36 bp, Artificial Sequence
  >BioblockX2_95
ctaagtagtcagagtctgactgactgagtcagggga

SEQ ID NO: 97 - DNA, 36 bp, Artificial Sequence
  >BioblockX2_96
aatagtagagtctgtgtgactgtgagtctcagtcaa

SEQ ID NO: 98 - DNA, 36 bp, Artificial Sequence
  >BioblockX2_97
tcaagtagacacacagtgacactctcactcagcttc

SEQ ID NO: 99 - DNA, 36 bp, Artificial Sequence
  >BioblockX2_98
cttcgtagagacacagtgactgtgtgtctcagagta

SEQ ID NO: 100 - DNA, 36 bp, Artificial Sequence
  >BioblockX2_99
agtagtagtgactctctgactctgtcagtcagactg

SEQ ID NO: 101 - DNA, 36 bp, Artificial Sequence
  >BioblockX2_100
actggtagactgtctctgactgtctgtctcagcaca

SEQ ID NO: 102 - DNA, 36 bp, Artificial Sequence
  >BioblockX2_101
cacagtagtgtctctctgacagtgtcagtcagccag

SEQ ID NO: 103 - DNA, 36 bp, Artificial Sequence
  >BioblockX2_102
ccaggtagactgacagtgactctctgagtcagcaaa

SEQ ID NO: 104 - DNA, 36 bp, Artificial Sequence
  >BioblockX2_103
caaagtagtgagactctgacagtgacactcaggacc

SEQ ID NO: 105 - DNA, 36 bp, Artificial Sequence
  >BioblockX2_104
gaccgtagagtcactgtgactcagtctgtcagactc

SEQ ID NO: 106 - DNA, 36 bp, Artificial Sequence
  >BioblockX2_105
actcgtagtcactgtctgacagtgtgactcagccac

SEQ ID NO: 107 - DNA, 36 bp, Artificial Sequence
  >BioblockX2_106
ccacgtagtgtgacagtgacagtgtgagtcaggaac

SEQ ID NO: 108 - DNA, 36 bp, Artificial Sequence
  >BioblockX2_107
gaacgtagtcagtgactgactgagagagtcaggcac

SEQ ID NO: 109 - DNA, 36 bp, Artificial Sequence
  >BioblockX2_108
gcacgtagagacagagtgacacagacactcagcggc

SEQ ID NO: 110 - DNA, 36 bp, Artificial Sequence
  >BioblockX2_109
cggcgtagtgagagtgtgacactgagactcagcgta

SEQ ID NO: 111 - DNA, 36 bp, Artificial Sequence
  >BioblockX2_110
cgtagtagacagtcactgactgagtctgtcaggtaa

SEQ ID NO: 112 - DNA, 36 bp, Artificial Sequence
  >BioblockX2_111
gtaagtagactgtcagtgactgactctgtcagcaac

SEQ ID NO: 113 - DNA, 36 bp, Artificial Sequence
  >BioblockX2_112
caacgtagtctctctgtgactgagacactcaggcta

SEQ ID NO: 114 - DNA, 36 bp, Artificial Sequence
  >BioblockX2_113
gctagtagtcagagactgacagtgtgactcagccga

SEQ ID NO: 115 - DNA, 36 bp, Artificial Sequence
  >BioblockX2_114
ccgagtagagacacagtgacagtgagtctcagacga

SEQ ID NO: 116 - DNA, 36 bp, Artificial Sequence
  >BioblockX2_115
acgagtagtgacagagtgactctgtcactcagagaa

SEQ ID NO: 117 - DNA, 36 bp, Artificial Sequence
  >BioblockX2_116
agaagtagtctctcactgactctgactctcagtaaa

SEQ ID NO: 118 - DNA, 36 bp, Artificial Sequence
  >BioblockX2_117
taaagtagtgagagtgtgactgtgtcactcagagcg

SEQ ID NO: 119 - DNA, 36 bp, Artificial Sequence
  >BioblockX2_118
agcggtagtcagacagtgactgtgtgtgtcagacct

SEQ ID NO: 120 - DNA, 36 bp, Artificial Sequence
  >BioblockX2_119
acctgtagagagactctgacagtgagagtcagaaca

SEQ ID NO: 121 - DNA, 36 bp, Artificial Sequence
  >BioblockX2_120
aacagtagagtctgtctgacactctcagtcagggca

SEQ ID NO: 122 - DNA, 36 bp, Artificial Sequence
  >BioblockX2_121
ggcagtagtctcagactgactgtctgagtcagacgc

SEQ ID NO: 123 - DNA, 36 bp, Artificial Sequence
  >BioblockX2_122
acgcgtagactcagtgtgacagacactctcagaatc

SEQ ID NO: 124 - DNA, 36 bp, Artificial Sequence
  >BioblockX2_123
aatcgtagtcacagtctgactgtgtcactcagcgag

SEQ ID NO: 125 - DNA, 36 bp, Artificial Sequence
  >BioblockX2_124
cgaggtagacactgagtgacactctgagtcagtcca

SEQ ID NO: 126 - DNA, 36 bp, Artificial Sequence
  >BioblockX2_125
tccagtagtcagagtctgactgtgacactcagccta

SEQ ID NO: 127 - DNA, 36 bp, Artificial Sequence
  >BioblockX2_126
cctagtagtgagacagtgacagtgtcactcagctaa

SEQ ID NO: 128 - DNA, 36 bp, Artificial Sequence
  >BioblockX2_127
ctaagtagacagagtgtgactgagtctgtcagggga

SEQ ID NO: 129 - DNA, 36 bp, Artificial Sequence
  >BioblockX2_128
aatagtagagtgacagtgactcagagactcagtcaa

SEQ ID NO: 130 - DNA, 36 bp, Artificial Sequence
  >BioblockX2_129
tcaagtagactctgagtgacactgtgtgtcagcttc

SEQ ID NO: 131 - DNA, 36 bp, Artificial Sequence
  >BioblockX2_130
cttcgtagtctgacactgactgtgacagtcagagta

SEQ ID NO: 132 - DNA, 36 bp, Artificial Sequence
  >BioblockX2_131
agtagtagtcagacagtgacagactgactcagactg

SEQ ID NO: 133 - DNA, 36 bp, Artificial Sequence
  >BioblockX2_132
actggtagagtcagactgacactcagactcagcaca

SEQ ID NO: 134 - DNA, 36 bp, Artificial Sequence
  >BioblockX2_133
cacagtagagacagagtgactgtctcactcagccag

SEQ ID NO: 135 - DNA, 36 bp, Artificial Sequence
  >BioblockX2_134
ccaggtagtgacagtgtgacagtgacagtcagcaaa

SEQ ID NO: 136 - DNA, 36 bp, Artificial Sequence
  >BioblockX2_135
caaagtagtgagagactgactctcactgtcaggacc

SEQ ID NO: 137 - DNA, 36 bp, Artificial Sequence
  >BioblockX2_136
gaccgtagtgtgtcagtgactcacactgtcagactc

SEQ ID NO: 138 - DNA, 36 bp, Artificial Sequence
  >BioblockX2_137
actcgtagtgtctctgtgacacactgtgtcagccac

SEQ ID NO: 139 - DNA, 36 bp, Artificial Sequence
  >BioblockX2_138
ccacgtagagtctcagtgactctcagactcaggaac

SEQ ID NO: 140 - DNA, 36 bp, Artificial Sequence
  >BioblockX2_139
gaacgtagagacactctgacagtgtgtctcaggcac

SEQ ID NO: 141 - DNA, 36 bp, Artificial Sequence
  >BioblockX2_140
gcacgtagagagagactgactctctcagtcagcggc

SEQ ID NO: 142 - DNA, 36 bp, Artificial Sequence
  >BioblockX2_141
cggcgtagagtcacagtgactgtgagagtcagcgta

SEQ ID NO: 143 - DNA, 36 bp, Artificial Sequence
  >BioblockX2_142
cgtagtagtgtcagtgtgactctgactctcaggtaa

SEQ ID NO: 144 - DNA, 36 bp, Artificial Sequence
  >BioblockX2_143
gtaagtagtgtgtgtctgacagtcacagtcagcaac

SEQ ID NO: 145 - DNA, 36 bp, Artificial Sequence
  >BioblockX2_144
caacgtagtcacactctgacagagacactcaggcta

SEQ ID NO: 146 - DNA, 36 bp, Artificial Sequence
  >BioblockX2_145
gctagtagacactgactgacactcactctcagccga

SEQ ID NO: 147 - DNA, 36 bp, Artificial Sequence
  >BioblockX2_146
ccgagtagagtgagagtgacagacagtgtcagacga

SEQ ID NO: 148 - DNA, 36 bp, Artificial Sequence
  >BioblockX2_147
acgagtagtgagactctgactgagtgagtcagagaa

SEQ ID NO: 149 - DNA, 36 bp, Artificial Sequence
  >BioblockX2_148
agaagtagtctgagtgtgacagtgtgagtcagtaaa

SEQ ID NO: 150 - DNA, 36 bp, Artificial Sequence
  >BioblockX2_149
taaagtagtctgagagtgacacagtgtctcagagcg

SEQ ID NO: 151 - DNA, 36 bp, Artificial Sequence
  >BioblockX2_150
agcggtagagagactgtgactctcagtgtcagacct

SEQ ID NO: 152 - DNA, 36 bp, Artificial Sequence
  >BioblockX2_151
acctgtagactctgagtgactctgtgtctcagaaca

SEQ ID NO: 153 - DNA, 36 bp, Artificial Sequence
  >BioblockX2_152
aacagtagtgagactctgactcagacactcagggca

SEQ ID NO: 154 - DNA, 36 bp, Artificial Sequence
  >BioblockX2_153
ggcagtagagtgagtctgacacactcagtcagacgc

SEQ ID NO: 155 - DNA, 36 bp, Artificial Sequence
  >BioblockX2_154
acgcgtagtgtgagtctgacagtctcactcagaatc

SEQ ID NO: 156 - DNA, 36 bp, Artificial Sequence
  >BioblockX2_155
aatcgtagagactgagtgactgagactctcagcgag

SEQ ID NO: 157 - DNA, 36 bp, Artificial Sequence
  >BioblockX2_156
cgaggtagtgacagtgtgactctgtctctcagtcca

SEQ ID NO: 158 - DNA, 36 bp, Artificial Sequence
  >BioblockX2_157
tccagtagagtgagtctgacagagagactcagccta

SEQ ID NO: 159 - DNA, 36 bp, Artificial Sequence
  >BioblockX2_158
cctagtagactctctgtgacacagagactcagctaa

SEQ ID NO: 160 - DNA, 36 bp, Artificial Sequence
  >BioblockX2_159
ctaagtagacacagactgacagagacagtcagggga

SEQ ID NO: 161 - DNA, 36 bp, Artificial Sequence
  >BioblockX2_160
aatagtagagtctgtctgactgtctgagtcagtcaa

SEQ ID NO: 162 - DNA, 36 bp, Artificial Sequence
  >BioblockX2_161
tcaagtagagagtgagtgacagtcacagtcagcttc

SEQ ID NO: 163 - DNA, 36 bp, Artificial Sequence
  >BioblockX2_162
cttcgtagtgagagagtgactgtcactgtcagagta

SEQ ID NO: 164 - DNA, 36 bp, Artificial Sequence
  >BioblockX2_163
agtagtagtctgtgagtgacactcacactcagactg

SEQ ID NO: 165 - DNA, 36 bp, Artificial Sequence
  >BioblockX2_164
actggtagtgagagactgactgtgtcagtcagcaca

SEQ ID NO: 166 - DNA, 36 bp, Artificial Sequence
  >BioblockX2_165
cacagtagtgactgagtgacactgacagtcagccag

SEQ ID NO: 167 - DNA, 36 bp, Artificial Sequence
  >BioblockX2_166
ccaggtagagagtgagtgactgagagactcagcaaa

SEQ ID NO: 168 - DNA, 36 bp, Artificial Sequence
  >BioblockX2_167
caaagtagacagagagtgacacacagactcaggacc

SEQ ID NO: 169 - DNA, 36 bp, Artificial Sequence
  >BioblockX2_168
gaccgtagtgagtcactgactgtgagactcagactc

SEQ ID NO: 170 - DNA, 36 bp, Artificial Sequence
  >BioblockX2_169
actcgtagtctgagtctgactgactctgtcagccac

SEQ ID NO: 171 - DNA, 36 bp, Artificial Sequence
  >BioblockX2_170
ccacgtagactcactctgacacactgagtcaggaac

SEQ ID NO: 172 - DNA, 36 bp, Artificial Sequence
  >BioblockX2_171
gaacgtagtcacacagtgactctctcactcaggcac

SEQ ID NO: 173 - DNA, 36 bp, Artificial Sequence
  >BioblockX2_172
gcacgtagagactgactgacagagacagtcagcggc

SEQ ID NO: 174 - DNA, 36 bp, Artificial Sequence
  >BioblockX2_173
cggcgtagtgagagtctgactctcagtctcagcgta

SEQ ID NO: 175 - DNA, 36 bp, Artificial Sequence
  >BioblockX2_174
cgtagtagacagagagtgacagactctgtcaggtaa

SEQ ID NO: 176 - DNA, 36 bp, Artificial Sequence
  >BioblockX2_175
gtaagtagtgactgagtgacactcactctcagcaac

SEQ ID NO: 177 - DNA, 36 bp, Artificial Sequence
  >BioblockX2_176
caacgtagtcactgactgactgtctcactcaggcta

SEQ ID NO: 178 - DNA, 36 bp, Artificial Sequence
  >BioblockX2_177
gctagtagtgtgacactgactcacacactcagccga

SEQ ID NO: 179 - DNA, 36 bp, Artificial Sequence
  >BioblockX2_178
ccgagtagtctgtgtctgactcactgagtcagacga

SEQ ID NO: 180 - DNA, 36 bp, Artificial Sequence
  >BioblockX2_179
acgagtagtctctgagtgactctgagactcagagaa

SEQ ID NO: 181 - DNA, 36 bp, Artificial Sequence
  >BioblockX2_180
agaagtagacacagtgtgactcactgactcagtaaa

SEQ ID NO: 182 - DNA, 36 bp, Artificial Sequence
  >BioblockX2_181
taaagtagagacagactgacactgtgactcagagcg

SEQ ID NO: 183 - DNA, 36 bp, Artificial Sequence
  >BioblockX2_182
agcggtagacacactctgactctctgactcagacct

SEQ ID NO: 184 - DNA, 36 bp, Artificial Sequence
  >BioblockX2_183
acctgtagacacagtctgacacacacactcagaaca

SEQ ID NO: 185 - DNA, 36 bp, Artificial Sequence
  >BioblockX2_184
aacagtagacagagactgactcacacagtcagggca

SEQ ID NO: 186 - DNA, 36 bp, Artificial Sequence
  >BioblockX2_185
ggcagtagacagagagtgacacagtcactcagacgc

SEQ ID NO: 187 - DNA, 36 bp, Artificial Sequence
  >BioblockX2_186
acgcgtagagactgtctgactcagactgtcagaatc

SEQ ID NO: 188 - DNA, 36 bp, Artificial Sequence
  >BioblockX2_187
aatcgtagtcacacactgacactgacagtcagcgag

SEQ ID NO: 189 - DNA, 36 bp, Artificial Sequence
  >BioblockX2_188
cgaggtagtgagtgtctgacacactcagtcagtcca

SEQ ID NO: 190 - DNA, 36 bp, Artificial Sequence
  >BioblockX2_189
tccagtagactcactctgactgagagagtcagccta

SEQ ID NO: 191 - DNA, 36 bp, Artificial Sequence
  >BioblockX2_190
cctagtagtcacacagtgactctgtcagtcagctaa

SEQ ID NO: 192 - DNA, 36 bp, Artificial Sequence
  >BioblockX2_191
ctaagtagacagagtgtgactgtcagtctcagggga

SEQ ID NO: 193 - DNA, 36 bp, Artificial Sequence
  >BioblockX2_192
aatagtagtctctcagtgactctgagagtcagtcaa

SEQ ID NO: 194 - DNA, 36 bp, Artificial Sequence
  >BioblockX2_193
tcaagtagtcagagtctgacactgtgtgtcagcttc

SEQ ID NO: 195 - DNA, 36 bp, Artificial Sequence
  >BioblockX2_194
cttcgtagagtctcactgacacagtgagtcagagta

SEQ ID NO: 196 - DNA, 36 bp, Artificial Sequence
  >BioblockX2_195
agtagtagtgagagagtgacagagagagtcagactg

SEQ ID NO: 197 - DNA, 36 bp, Artificial Sequence
  >BioblockX2_196
actggtagtgacagactgacagactgagtcagcaca

SEQ ID NO: 198 - DNA, 36 bp, Artificial Sequence
  >BioblockX2_197
cacagtagtcagacactgacagtgagagtcagccag

SEQ ID NO: 199 - DNA, 36 bp, Artificial Sequence
  >BioblockX2_198
ccaggtagtgtcagtgtgacagacacactcagcaaa

SEQ ID NO: 200 - DNA, 36 bp, Artificial Sequence
  >BioblockX2_199
caaagtagagtctctctgacactgtgagtcaggacc

SEQ ID NO: 201 - DNA, 36 bp, Artificial Sequence
  >BioblockX2_200
gaccgtagtctcactctgactcagactctcagactc

SEQ ID NO: 202 - DNA, 36 bp, Artificial Sequence
  >BioblockX2_201
actcgtagtgactgagtgactgactcagtcagccac

SEQ ID NO: 203 - DNA, 36 bp, Artificial Sequence
  >BioblockX2_202
ccacgtagtcacagtgtgactgagagagtcaggaac

SEQ ID NO: 204 - DNA, 36 bp, Artificial Sequence
  >BioblockX2_203
gaacgtagtgtcagtctgactgtgtctgtcaggcac

SEQ ID NO: 205 - DNA, 36 bp, Artificial Sequence
  >BioblockX2_204
gcacgtagtcagagtctgacagagtgagtcagcggc

SEQ ID NO: 206 - DNA, 36 bp, Artificial Sequence
  >BioblockX2_205
cggcgtagtcagagtctgacactctgagtcagcgta

SEQ ID NO: 207 - DNA, 36 bp, Artificial Sequence
  >BioblockX2_206
cgtagtagtctgagactgacacactcactcaggtaa

SEQ ID NO: 208 - DNA, 36 bp, Artificial Sequence
  >BioblockX2_207
gtaagtagtgtgagactgactgtcacagtcagcaac

SEQ ID NO: 209 - DNA, 36 bp, Artificial Sequence
  >BioblockX2_208
caacgtagtctgactgtgacacagagtctcaggcta

SEQ ID NO: 210 - DNA, 36 bp, Artificial Sequence
  >BioblockX2_209
gctagtagacagactgtgactgactgtctcagccga

SEQ ID NO: 211 - DNA, 36 bp, Artificial Sequence
  >BioblockX2_210
ccgagtagtctcagtctgactgagtcactcagacga

SEQ ID NO: 212 - DNA, 36 bp, Artificial Sequence
  >BioblockX2_211
acgagtagtgagacactgacactgtgactcagagaa

SEQ ID NO: 213 - DNA, 36 bp, Artificial Sequence
  >BioblockX2_212
agaagtagtctgtctgtgacactcacactcagtaaa

SEQ ID NO: 214 - DNA, 36 bp, Artificial Sequence
  >BioblockX2_213
taaagtagtgtcagtgtgacagagtgagtcagagcg

SEQ ID NO: 215 - DNA, 36 bp, Artificial Sequence
  >BioblockX2_214
agcggtagtcacacactgactcactctgtcagacct

SEQ ID NO: 216 - DNA, 36 bp, Artificial Sequence
  >BioblockX2_215
acctgtagtgactctctgactgactgtctcagaaca

SEQ ID NO: 217 - DNA, 36 bp, Artificial Sequence
  >BioblockX2_216
aacagtagacagtgtgtgacacacacagtcagggca

SEQ ID NO: 218 - DNA, 36 bp, Artificial Sequence
  >BioblockX2_217
ggcagtagtctgagagtgactgtgtcagtcagacgc

SEQ ID NO: 219 - DNA, 36 bp, Artificial Sequence
  >BioblockX2_218
acgcgtagtctgagactgacactgtcactcagaatc

SEQ ID NO: 220 - DNA, 36 bp, Artificial Sequence
  >BioblockX2_219
aatcgtagactgtgagtgacagtgagactcagcgag

SEQ ID NO: 221 - DNA, 36 bp, Artificial Sequence
  >BioblockX2_220
cgaggtagtgtcacactgacactcactctcagtcca

SEQ ID NO: 222 - DNA, 36 bp, Artificial Sequence
  >BioblockX2_221
tccagtagtgtgtgagtgactctcagtctcagccta

SEQ ID NO: 223 - DNA, 36 bp, Artificial Sequence
  >BioblockX2_222
cctagtagacacacactgacacagtgtctcagctaa

SEQ ID NO: 224 - DNA, 36 bp, Artificial Sequence
  >BioblockX2_223
ctaagtagtgagagtctgactcactctgtcagggga

SEQ ID NO: 225 - DNA, 36 bp, Artificial Sequence
  >BioblockX2_224
aatagtagactgtgtctgactgtgtgagtcagtcaa

SEQ ID NO: 226 - DNA, 36 bp, Artificial Sequence
  >BioblockX2_225
tcaagtagactgacagtgactcacagactcagcttc

SEQ ID NO: 227 - DNA, 36 bp, Artificial Sequence
  >BioblockX2_226
cttcgtagtgagtgagtgactgtgagtgtcagagta

SEQ ID NO: 228 - DNA, 36 bp, Artificial Sequence
  >BioblockX2_227
agtagtagtctcagactgactgtgtgagtcagactg

SEQ ID NO: 229 - DNA, 36 bp, Artificial Sequence
  >BioblockX2_228
actggtagactcacactgacactgactctcagcaca

SEQ ID NO: 230 - DNA, 36 bp, Artificial Sequence
  >BioblockX2_229
cacagtagacacacagtgactcagtcactcagccag

SEQ ID NO: 231 - DNA, 36 bp, Artificial Sequence
  >BioblockX2_230
ccaggtagagtgtcactgactcactctctcagcaaa

SEQ ID NO: 232 - DNA, 36 bp, Artificial Sequence
  >BioblockX2_231
caaagtagtcactgactgacactcagtctcaggacc

SEQ ID NO: 233 - DNA, 36 bp, Artificial Sequence
  >BioblockX2_232
gaccgtagtctgtgagtgacactcacactcagactc

SEQ ID NO: 234 - DNA, 36 bp, Artificial Sequence
  >BioblockX2_233
actcgtagagagactgtgacacagtgagtcagccac

SEQ ID NO: 235 - DNA, 36 bp, Artificial Sequence
  >BioblockX2_234
ccacgtagtctcacagtgacactgtgtctcaggaac

SEQ ID NO: 236 - DNA, 36 bp, Artificial Sequence
  >BioblockX2_235
gaacgtagagtctgagtgacagtctgactcaggcac

SEQ ID NO: 237 - DNA, 36 bp, Artificial Sequence
  >BioblockX2_236
gcacgtagtcagtctgtgactctctctgtcagcggc

SEQ ID NO: 238 - DNA, 36 bp, Artificial Sequence
  >BioblockX2_237
cggcgtagagtgtctctgactgagtgagtcagcgta

SEQ ID NO: 239 - DNA, 36 bp, Artificial Sequence
  >BioblockX2_238
cgtagtagtcagacagtgacactgagagtcaggtaa

SEQ ID NO: 240 - DNA, 36 bp, Artificial Sequence
  >BioblockX2_239
gtaagtagtgtgagactgacagtcagagtcagcaac

SEQ ID NO: 241 - DNA, 36 bp, Artificial Sequence
  >BioblockX2_240
caacgtagtgactcagtgacagagtctctcaggcta

SEQ ID NO: 242 - DNA, 36 bp, Artificial Sequence
  >BioblockX2_241
gctagtagtctgtcactgactgtctcagtcagccga

SEQ ID NO: 243 - DNA, 36 bp, Artificial Sequence
  >BioblockX2_242
ccgagtagtgtgtgagtgacagtctctctcagacga

SEQ ID NO: 244 - DNA, 36 bp, Artificial Sequence
  >BioblockX2_243
acgagtagagactgtctgactgagactgtcagagaa

SEQ ID NO: 245 - DNA, 36 bp, Artificial Sequence
  >BioblockX2_244
agaagtagtgactcagtgactgacactctcagtaaa

SEQ ID NO: 246 - DNA, 36 bp, Artificial Sequence
  >BioblockX2_245
taaagtagacagactgtgacactgtcactcagagcg

SEQ ID NO: 247 - DNA, 36 bp, Artificial Sequence
  >BioblockX2_246
agcggtagtcactcagtgacacactctgtcagacct

SEQ ID NO: 248 - DNA, 36 bp, Artificial Sequence
  >BioblockX2_247
acctgtagtgtctgtgtgactgtctctctcagaaca

SEQ ID NO: 249 - DNA, 36 bp, Artificial Sequence
  >BioblockX2_248
aacagtagacactgtctgactgacagtgtcagggca

SEQ ID NO: 250 - DNA, 36 bp, Artificial Sequence
  >BioblockX2_249
ggcagtagtctctctgtgacacagtctgtcagacgc

SEQ ID NO: 251 - DNA, 36 bp, Artificial Sequence
  >BioblockX2_250
acgcgtagtcacagagtgactgactctctcagaatc

SEQ ID NO: 252 - DNA, 36 bp, Artificial Sequence
  >BioblockX2_251
aatcgtagactgactgtgacacagtctgtcagcgag

SEQ ID NO: 253 - DNA, 36 bp, Artificial Sequence
  >BioblockX2_252
cgaggtagtgagacactgacagtgagtgtcagtcca

SEQ ID NO: 254 - DNA, 36 bp, Artificial Sequence
  >BioblockX2_253
tccagtagagtctgagtgactctcagactcagccta

SEQ ID NO: 255 - DNA, 36 bp, Artificial Sequence
  >BioblockX2_254
cctagtagtctgtgtgtgactgtgagtgtcagctaa

SEQ ID NO: 256 - DNA, 36 bp, Artificial Sequence
  >BioblockX2_255
ctaagtagtcactgtctgacagactgtctcagggga

SEQ ID NO: 257 - DNA, 36 bp, Artificial Sequence
  >BioblockX2_256
aatagtagagtctctctgacagtcagactcagtcaa

SEQ ID NO: 258 - DNA, 36 bp, Artificial Sequence
  >BioblockX2_257
tcaagtagtcacacagtgactcactctctcagcttc

SEQ ID NO: 259 - DNA, 36 bp, Artificial Sequence
  >BioblockX2_258
cttcgtagagtctgtgtgacagagtgagtcagagta

SEQ ID NO: 260 - DNA, 36 bp, Artificial Sequence
  >BioblockX2_259
agtagtagtgactctgtgacacactgtctcagactg

SEQ ID NO: 261 - DNA, 36 bp, Artificial Sequence
  >BioblockX2_260
actggtagagtgtctctgactgactgagtcagcaca

SEQ ID NO: 262 - DNA, 36 bp, Artificial Sequence
  >BioblockX2_261
cacagtagtgtctctgtgacactcacagtcagccag

SEQ ID NO: 263 - DNA, 36 bp, Artificial Sequence
  >BioblockX2_262
ccaggtagtgagtctgtgactgactgtgtcagcaaa

SEQ ID NO: 264 - DNA, 36 bp, Artificial Sequence
  >BioblockX2_263
caaagtagtctgagagtgacagtcacactcaggacc

SEQ ID NO: 265 - DNA, 36 bp, Artificial Sequence
  >BioblockX2_264
gaccgtagtcagtctgtgacacacactctcagactc

SEQ ID NO: 266 - DNA, 36 bp, Artificial Sequence
  >BioblockX2_265
actcgtagtctctgagtgactgagtgtctcagccac

SEQ ID NO: 267 - DNA, 36 bp, Artificial Sequence
  >BioblockX2_266
ccacgtagactcacactgacagtctgactcaggaac

SEQ ID NO: 268 - DNA, 36 bp, Artificial Sequence
  >BioblockX2_267
gaacgtagactgacactgactcagtgagtcaggcac

SEQ ID NO: 269 - DNA, 36 bp, Artificial Sequence
  >BioblockX2_268
gcacgtagtgactcactgactcacacactcagcggc

SEQ ID NO: 270 - DNA, 36 bp, Artificial Sequence
  >BioblockX2_269
cggcgtagtctgtgtgtgactctgagtgtcagcgta

SEQ ID NO: 271 - DNA, 36 bp, Artificial Sequence
  >BioblockX2_270
cgtagtagtgacactgtgactgtctctgtcaggtaa

SEQ ID NO: 272 - DNA, 36 bp, Artificial Sequence
  >BioblockX2_271
gtaagtagtcagacactgacactgtcactcagcaac

SEQ ID NO: 273 - DNA, 36 bp, Artificial Sequence
  >BioblockX2_272
caacgtagactcagtgtgacactgacagtcaggcta

SEQ ID NO: 274 - DNA, 36 bp, Artificial Sequence
  >BioblockX2_273
gctagtagtgacagagtgactctctgagtcagccga

SEQ ID NO: 275 - DNA, 36 bp, Artificial Sequence
  >BioblockX2_274
ccgagtagactcagagtgacagagacagtcagacga

SEQ ID NO: 276 - DNA, 36 bp, Artificial Sequence
  >BioblockX2_275
acgagtagacagacagtgactgagtgactcagagaa

SEQ ID NO: 277 - DNA, 36 bp, Artificial Sequence
  >BioblockX2_276
agaagtagtgagtcactgacagtcagtgtcagtaaa

SEQ ID NO: 278 - DNA, 36 bp, Artificial Sequence
  >BioblockX2_277
taaagtagtgactgactgactgtcacagtcagagcg

SEQ ID NO: 279 - DNA, 36 bp, Artificial Sequence
  >BioblockX2_278
agcggtagacagagactgactgagtgactcagacct

SEQ ID NO: 280 - DNA, 36 bp, Artificial Sequence
  >BioblockX2_279
acctgtagagagacactgactgtcacagtcagaaca

SEQ ID NO: 281 - DNA, 36 bp, Artificial Sequence
  >BioblockX2_280
aacagtagtcagacagtgacagagtcagtcagggca

SEQ ID NO: 282 - DNA, 36 bp, Artificial Sequence
  >BioblockX2_281
ggcagtagtctcacagtgactctctctgtcagacgc

SEQ ID NO: 283 - DNA, 36 bp, Artificial Sequence
  >BioblockX2_282
acgcgtagacagtcagtgactgtgactgtcagaatc

SEQ ID NO: 284 - DNA, 36 bp, Artificial Sequence
  >BioblockX2_283
aatcgtagtgtctcagtgactcactcagtcagcgag

SEQ ID NO: 285 - DNA, 36 bp, Artificial Sequence
  >BioblockX2_284
cgaggtagactgacagtgacactgtctctcagtcca

SEQ ID NO: 286 - DNA, 36 bp, Artificial Sequence
  >BioblockX2_285
tccagtagtcagagagtgacagtcacagtcagccta

SEQ ID NO: 287 - DNA, 36 bp, Artificial Sequence
  >BioblockX2_286
cctagtagtgtcactctgactgtgtgtgtcagctaa

SEQ ID NO: 288 - DNA, 36 bp, Artificial Sequence
  >BioblockX2_287
ctaagtagtgagtcactgactgagagtctcagggga

SEQ ID NO: 289 - DNA, 36 bp, Artificial Sequence
  >BioblockX2_288
aatagtagtgagtctgtgacacactcagtcagtcaa

SEQ ID NO: 290 - DNA, 36 bp, Artificial Sequence
  >BioblockX2_289
tcaagtagactgtgtgtgacactgtgtgtcagcttc

SEQ ID NO: 291 - DNA, 36 bp, Artificial Sequence
  >BioblockX2_290
cttcgtagtcactctctgacacacagtgtcagagta

SEQ ID NO: 292 - DNA, 36 bp, Artificial Sequence
  >BioblockX2_291
agtagtagagagacactgacagacactctcagactg

SEQ ID NO: 293 - DNA, 36 bp, Artificial Sequence
  >BioblockX2_292
actggtagtctgtgtgtgactgtctctgtcagcaca

SEQ ID NO: 294 - DNA, 36 bp, Artificial Sequence
  >BioblockX2_293
cacagtagtctcagagtgacagtcactgtcagccag

SEQ ID NO: 295 - DNA, 36 bp, Artificial Sequence
  >BioblockX2_294
ccaggtagtcagacagtgactgacagtctcagcaaa

SEQ ID NO: 296 - DNA, 36 bp, Artificial Sequence
  >BioblockX2_295
caaagtagagtctgtgtgacactcactgtcaggacc

SEQ ID NO: 297 - DNA, 36 bp, Artificial Sequence
  >BioblockX2_296
gaccgtagtgtgacagtgacacagtctctcagactc

SEQ ID NO: 298 - DNA, 36 bp, Artificial Sequence
  >BioblockX2_297
actcgtagagacactgtgacacagtctgtcagccac

SEQ ID NO: 299 - DNA, 36 bp, Artificial Sequence
  >BioblockX2_298
ccacgtagagactcactgacactgtgagtcaggaac

SEQ ID NO: 300 - DNA, 36 bp, Artificial Sequence
  >BioblockX2_299
gaacgtagacactgtctgactcagtcagtcaggcac

SEQ ID NO: 301 - DNA, 36 bp, Artificial Sequence
  >BioblockX2_300
gcacgtagagagagtctgacagactgagtcagcggc

SEQ ID NO: 302 - DNA, 36 bp, Artificial Sequence
  >BioblockX2_301
cggcgtagtctgtctgtgacactgtgtctcagcgta

SEQ ID NO: 303 - DNA, 36 bp, Artificial Sequence
  >BioblockX2_302
cgtagtagacactgtctgacagtctgactcaggtaa

SEQ ID NO: 304 - DNA, 36 bp, Artificial Sequence
  >BioblockX2_303
gtaagtagagagacagtgacagtgagtgtcagcaac

SEQ ID NO: 305 - DNA, 36 bp, Artificial Sequence
  >BioblockX2_304
caacgtagagagactctgacagtgagagtcaggcta

SEQ ID NO: 306 - DNA, 36 bp, Artificial Sequence
  >BioblockX2_305
gctagtagagtgagagtgacacacacagtcagccga

SEQ ID NO: 307 - DNA, 36 bp, Artificial Sequence
  >BioblockX2_306
ccgagtagtgacagtctgacactctctgtcagacga

SEQ ID NO: 308 - DNA, 36 bp, Artificial Sequence
  >BioblockX2_307
acgagtagtgacacactgactgtgtctctcagagaa

SEQ ID NO: 309 - DNA, 36 bp, Artificial Sequence
  >BioblockX2_308
agaagtagtgtgtgagtgacacactgtgtcagtaaa

SEQ ID NO: 310 - DNA, 36 bp, Artificial Sequence
  >BioblockX2_309
taaagtagacagagtgtgacacactcactcagagcg

SEQ ID NO: 311 - DNA, 36 bp, Artificial Sequence
  >BioblockX2_310
agcggtagtgtcagtgtgacagtctctgtcagacct

SEQ ID NO: 312 - DNA, 36 bp, Artificial Sequence
  >BioblockX2_311
acctgtagagactcagtgactctgtgactcagaaca

SEQ ID NO: 313 - DNA, 36 bp, Artificial Sequence
  >BioblockX2_312
aacagtagtctgagtctgacagtctgactcagggca

SEQ ID NO: 314 - DNA, 36 bp, Artificial Sequence
  >BioblockX2_313
ggcagtagacagactctgacagactcagtcagacgc

SEQ ID NO: 315 - DNA, 36 bp, Artificial Sequence
  >BioblockX2_314
acgcgtagactgtgtctgactcagacagtcagaatc

SEQ ID NO: 316 - DNA, 36 bp, Artificial Sequence
  >BioblockX2_315
aatcgtagactcacactgactctgactgtcagcgag

SEQ ID NO: 317 - DNA, 36 bp, Artificial Sequence
  >BioblockX2_316
cgaggtagagtcagagtgacacagtcagtcagtcca

SEQ ID NO: 318 - DNA, 36 bp, Artificial Sequence
  >BioblockX2_317
tccagtagtgacagagtgactgtctctctcagccta

SEQ ID NO: 319 - DNA, 36 bp, Artificial Sequence
  >BioblockX2_318
cctagtagagtgtgactgacacagtgactcagctaa

SEQ ID NO: 320 - DNA, 36 bp, Artificial Sequence
  >BioblockX2_319
ctaagtagagtcactgtgacagtgtgactcagggga

SEQ ID NO: 321 - DNA, 36 bp, Artificial Sequence
  >BioblockX2_320
aatagtagactcagtgtgacactgagagtcagtcaa

SEQ ID NO: 322 - DNA, 36 bp, Artificial Sequence
  >BioblockX2_321
tcaagtagagtgagtgtgactgtgagagtcagcttc

SEQ ID NO: 323 - DNA, 36 bp, Artificial Sequence
  >BioblockX2_322
cttcgtagagacagagtgactgtgacactcagagta

SEQ ID NO: 324 - DNA, 36 bp, Artificial Sequence
  >BioblockX2_323
agtagtagagacagagtgactcagtgtgtcagactg

SEQ ID NO: 325 - DNA, 36 bp, Artificial Sequence
  >BioblockX2_324
actggtagactgacactgacacacagagtcagcaca

SEQ ID NO: 326 - DNA, 36 bp, Artificial Sequence
  >BioblockX2_325
cacagtagagactgtgtgacacacacagtcagccag

SEQ ID NO: 327 - DNA, 36 bp, Artificial Sequence
  >BioblockX2_326
ccaggtagtgactgagtgacacacacactcagcaaa

SEQ ID NO: 328 - DNA, 36 bp, Artificial Sequence
  >BioblockX2_327
caaagtagagtgacagtgacacagacagtcaggacc

SEQ ID NO: 329 - DNA, 36 bp, Artificial Sequence
  >BioblockX2_328
gaccgtagtctcagtgtgactctctctctcagactc

SEQ ID NO: 330 - DNA, 36 bp, Artificial Sequence
  >BioblockX2_329
actcgtagagagactctgactgtgtgagtcagccac

SEQ ID NO: 331 - DNA, 36 bp, Artificial Sequence
  >BioblockX2_330
ccacgtagtgtgactgtgactgagtgtgtcaggaac

SEQ ID NO: 332 - DNA, 36 bp, Artificial Sequence
  >BioblockX2_331
gaacgtagagagtcagtgacactctgtctcaggcac

SEQ ID NO: 333 - DNA, 36 bp, Artificial Sequence
  >BioblockX2_332
gcacgtagtctgtctgtgacacagagactcagcggc

SEQ ID NO: 334 - DNA, 36 bp, Artificial Sequence
  >BioblockX2_333
cggcgtagtgtgtgactgacagtcactctcagcgta

SEQ ID NO: 335 - DNA, 36 bp, Artificial Sequence
  >BioblockX2_334
cgtagtagagactcagtgacacagtcagtcaggtaa

SEQ ID NO: 336 - DNA, 36 bp, Artificial Sequence
  >BioblockX2_335
gtaagtagagactgtgtgacactgagtctcagcaac

SEQ ID NO: 337 - DNA, 36 bp, Artificial Sequence
  >BioblockX2_336
caacgtagagagtctgtgactgtgtctgtcaggcta

SEQ ID NO: 338 - DNA, 36 bp, Artificial Sequence
  >BioblockX2_337
gctagtagtcagtgtgtgactcacagtgtcagccga

SEQ ID NO: 339 - DNA, 36 bp, Artificial Sequence
  >BioblockX2_338
ccgagtagacagagactgactcagtgtctcagacga

SEQ ID NO: 340 - DNA, 36 bp, Artificial Sequence
  >BioblockX2_339
acgagtagagtctctctgacacagagagtcagagaa

SEQ ID NO: 341 - DNA, 36 bp, Artificial Sequence
  >BioblockX2_340
agaagtagacagtgactgacagagacagtcagtaaa

SEQ ID NO: 342 - DNA, 36 bp, Artificial Sequence
  >BioblockX2_341
taaagtagagtgacactgacagtctgactcagagcg

SEQ ID NO: 343 - DNA, 36 bp, Artificial Sequence
  >BioblockX2_342
agcggtagtgacagagtgactgacagagtcagacct

SEQ ID NO: 344 - DNA, 36 bp, Artificial Sequence
  >BioblockX2_343
acctgtagtgtgactctgactgactgactcagaaca

SEQ ID NO: 345 - DNA, 36 bp, Artificial Sequence
  >BioblockX2_344
aacagtagtcagagtctgactcactgagtcagggca

SEQ ID NO: 346 - DNA, 36 bp, Artificial Sequence
  >BioblockX2_345
ggcagtagactgagagtgacactctctgtcagacgc

SEQ ID NO: 347 - DNA, 36 bp, Artificial Sequence
  >BioblockX2_346
acgcgtagtctcacagtgactgtctctgtcagaatc

SEQ ID NO: 348 - DNA, 36 bp, Artificial Sequence
  >BioblockX2_347
aatcgtagtgtctgactgacactgtctgtcagcgag

SEQ ID NO: 349 - DNA, 36 bp, Artificial Sequence
  >BioblockX2_348
cgaggtagtgacagactgactctgagagtcagtcca

SEQ ID NO: 350 - DNA, 36 bp, Artificial Sequence
  >BioblockX2_349
tccagtagagagagactgacagagtcagtcagccta

SEQ ID NO: 351 - DNA, 36 bp, Artificial Sequence
  >BioblockX2_350
cctagtagtgtctgagtgactgagtctgtcagctaa

SEQ ID NO: 352 - DNA, 36 bp, Artificial Sequence
  >BioblockX2_351
ctaagtagactctgagtgacacagtgtctcagggga

SEQ ID NO: 353 - DNA, 36 bp, Artificial Sequence
  >BioblockX2_352
aatagtagtctcagagtgacagagagtgtcagtcaa

SEQ ID NO: 354 - DNA, 36 bp, Artificial Sequence
  >BioblockX2_353
tcaagtagtctgtgactgacagtgagtgtcagcttc

SEQ ID NO: 355 - DNA, 36 bp, Artificial Sequence
  >BioblockX2_354
cttcgtagacagtcactgacagtcagtgtcagagta

SEQ ID NO: 356 - DNA, 36 bp, Artificial Sequence
  >BioblockX2_355
agtagtagacacactgtgacacactctgtcagactg

SEQ ID NO: 357 - DNA, 36 bp, Artificial Sequence
  >BioblockX2_356
actggtagactgtctgtgactcagtgagtcagcaca

SEQ ID NO: 358 - DNA, 36 bp, Artificial Sequence
  >BioblockX2_357
cacagtagtgtgagtgtgactgactgtgtcagccag

SEQ ID NO: 359 - DNA, 36 bp, Artificial Sequence
  >BioblockX2_358
ccaggtagagtgtcagtgactctctgagtcagcaaa

SEQ ID NO: 360 - DNA, 36 bp, Artificial Sequence
  >BioblockX2_359
caaagtagactcagactgactgacagtgtcaggacc

SEQ ID NO: 361 - DNA, 36 bp, Artificial Sequence
  >BioblockX2_360
gaccgtagagtcacagtgacactctcactcagactc

SEQ ID NO: 362 - DNA, 36 bp, Artificial Sequence
  >BioblockX2_361
actcgtagtcagtctgtgacacactctgtcagccac

SEQ ID NO: 363 - DNA, 36 bp, Artificial Sequence
  >BioblockX2_362
ccacgtagtctgagagtgactcactgtctcaggaac

SEQ ID NO: 364 - DNA, 36 bp, Artificial Sequence
  >BioblockX2_363
gaacgtagtgtgtcactgactgagacagtcaggcac

SEQ ID NO: 365 - DNA, 36 bp, Artificial Sequence
  >BioblockX2_364
gcacgtagtgagtctgtgactgtctgtgtcagcggc

SEQ ID NO: 366 - DNA, 36 bp, Artificial Sequence
  >BioblockX2_365
cggcgtagtcactcagtgactgtgtgagtcagcgta

SEQ ID NO: 367 - DNA, 36 bp, Artificial Sequence
  >BioblockX2_366
cgtagtagacagtgagtgacactgactctcaggtaa

SEQ ID NO: 368 - DNA, 36 bp, Artificial Sequence
  >BioblockX2_367
gtaagtagtcactcactgactgacagactcagcaac

SEQ ID NO: 369 - DNA, 36 bp, Artificial Sequence
  >BioblockX2_368
caacgtagtctctcagtgactgagtcagtcaggcta

SEQ ID NO: 370 - DNA, 36 bp, Artificial Sequence
  >BioblockX2_369
gctagtagacagtgactgactcagtgagtcagccga

SEQ ID NO: 371 - DNA, 36 bp, Artificial Sequence
  >BioblockX2_370
ccgagtagtgagtgagtgactctctgtctcagacga

SEQ ID NO: 372 - DNA, 36 bp, Artificial Sequence
  >BioblockX2_371
acgagtagtgactctctgacagagacagtcagagaa

SEQ ID NO: 373 - DNA, 36 bp, Artificial Sequence
  >BioblockX2_372
agaagtagtgtgtcactgactgtcactgtcagtaaa

SEQ ID NO: 374 - DNA, 36 bp, Artificial Sequence
  >BioblockX2_373
taaagtagtcagacagtgacagtgactgtcagagcg

SEQ ID NO: 375 - DNA, 36 bp, Artificial Sequence
  >BioblockX2_374
agcggtagtctcacactgactgtgtgactcagacct

SEQ ID NO: 376 - DNA, 36 bp, Artificial Sequence
  >BioblockX2_375
acctgtagagtgacagtgacagagacagtcagaaca

SEQ ID NO: 377 - DNA, 36 bp, Artificial Sequence
  >BioblockX2_376
aacagtagtgtcagactgacagtctgtctcagggca

SEQ ID NO: 378 - DNA, 36 bp, Artificial Sequence
  >BioblockX2_377
ggcagtagtgagagactgacagactctctcagacgc

SEQ ID NO: 379 - DNA, 36 bp, Artificial Sequence
  >BioblockX2_378
acgcgtagagtgacagtgactgactgagtcagaatc

SEQ ID NO: 380 - DNA, 36 bp, Artificial Sequence
  >BioblockX2_379
aatcgtagtcagtgagtgacacagtgtctcagcgag

SEQ ID NO: 381 - DNA, 36 bp, Artificial Sequence
  >BioblockX2_380
cgaggtagtgacagtctgacacactcactcagtcca

SEQ ID NO: 382 - DNA, 36 bp, Artificial Sequence
  >BioblockX2_381
tccagtagactgactgtgactgtgtcactcagccta

SEQ ID NO: 383 - DNA, 36 bp, Artificial Sequence
  >BioblockX2_382
cctagtagagtgagactgactgtgagtctcagctaa

SEQ ID NO: 384 - DNA, 36 bp, Artificial Sequence
  >BioblockX2_383
ctaagtagagacacactgacagacacactcagggga

SEQ ID NO: 385 - DNA, 36 bp, Artificial Sequence
  >BioblockX2_384
aatagtagtgtctgactgactgtgtgtctcagtcaa

SEQ ID NO: 386 - DNA, 36 bp, Artificial Sequence
  >BioblockX2_385
tcaagtagtcagagtgtgactctgagtctcagcttc

SEQ ID NO: 387 - DNA, 36 bp, Artificial Sequence
  >BioblockX2_386
cttcgtagagacagtctgacagactcactcagagta

SEQ ID NO: 388 - DNA, 36 bp, Artificial Sequence
  >BioblockX2_387
agtagtagtgtgtctctgacacacactctcagactg

SEQ ID NO: 389 - DNA, 36 bp, Artificial Sequence
  >BioblockX2_388
actggtagtctgacagtgactgacactctcagcaca

SEQ ID NO: 390 - DNA, 36 bp, Artificial Sequence
  >BioblockX2_389
cacagtagtgacacactgacagacagtgtcagccag

SEQ ID NO: 391 - DNA, 36 bp, Artificial Sequence
  >BioblockX2_390
ccaggtagtctgagactgacagtgacagtcagcaaa

SEQ ID NO: 392 - DNA, 36 bp, Artificial Sequence
  >BioblockX2_391
caaagtagagactcactgactcagactctcaggacc

SEQ ID NO: 393 - DNA, 36 bp, Artificial Sequence
  >BioblockX2_392
gaccgtagacagagtgtgactcagtctgtcagactc

SEQ ID NO: 394 - DNA, 36 bp, Artificial Sequence
  >BioblockX2_393
actcgtagagtcagtctgacacagtctctcagccac

SEQ ID NO: 395 - DNA, 36 bp, Artificial Sequence
  >BioblockX2_394
ccacgtagtgagactgtgactcacactgtcaggaac

SEQ ID NO: 396 - DNA, 36 bp, Artificial Sequence
  >BioblockX2_395
gaacgtagtcagacactgactgtgtcactcaggcac

SEQ ID NO: 397 - DNA, 36 bp, Artificial Sequence
  >BioblockX2_396
gcacgtagacagactctgactgtcactgtcagcggc

SEQ ID NO: 398 - DNA, 36 bp, Artificial Sequence
  >BioblockX2_397
cggcgtagtgtgtctctgactgagtgtctcagcgta

SEQ ID NO: 399 - DNA, 36 bp, Artificial Sequence
  >BioblockX2_398
cgtagtagtcagtctgtgacagtcagtgtcaggtaa

SEQ ID NO: 400 - DNA, 36 bp, Artificial Sequence
  >BioblockX2_399
gtaagtagtgactcagtgactgtgtctgtcagcaac

SEQ ID NO: 401 - DNA, 36 bp, Artificial Sequence
  >BioblockX2_400
caacgtagactctcactgacagtcagtgtcaggcta

SEQ ID NO: 402 - DNA, 36 bp, Artificial Sequence
  >BioblockX2_401
gctagtagtcagagagtgactgacacagtcagccga

SEQ ID NO: 403 - DNA, 36 bp, Artificial Sequence
  >BioblockX2_402
ccgagtagacacagagtgacagtgagtctcagacga

SEQ ID NO: 404 - DNA, 36 bp, Artificial Sequence
  >BioblockX2_403
acgagtagtcactcactgactcactcagtcagagaa

SEQ ID NO: 405 - DNA, 36 bp, Artificial Sequence
  >BioblockX2_404
agaagtagtcactcactgactcagacagtcagtaaa

SEQ ID NO: 406 - DNA, 36 bp, Artificial Sequence
  >BioblockX2_405
taaagtagacactctgtgacactgacactcagagcg

SEQ ID NO: 407 - DNA, 36 bp, Artificial Sequence
  >BioblockX2_406
agcggtagtgacagtgtgacactcactgtcagacct

SEQ ID NO: 408 - DNA, 36 bp, Artificial Sequence
  >BioblockX2_407
acctgtagtgtcacagtgacacacacagtcagaaca

SEQ ID NO: 409 - DNA, 36 bp, Artificial Sequence
  >BioblockX2_408
aacagtagacactctctgactgtctctgtcagggca

SEQ ID NO: 410 - DNA, 36 bp, Artificial Sequence
  >BioblockX2_409
ggcagtagagtgtgactgactcactcactcagacgc

SEQ ID NO: 411 - DNA, 36 bp, Artificial Sequence
  >BioblockX2_410
acgcgtagtgagagtgtgacagagagagtcagaatc

SEQ ID NO: 412 - DNA, 36 bp, Artificial Sequence
  >BioblockX2_411
aatcgtagagtgacactgacactgactctcagcgag

SEQ ID NO: 413 - DNA, 36 bp, Artificial Sequence
  >BioblockX2_412
cgaggtagagagtgtgtgactgacacactcagtcca

SEQ ID NO: 414 - DNA, 36 bp, Artificial Sequence
  >BioblockX2_413
tccagtagtgtgtgtgtgactcagtgagtcagccta

SEQ ID NO: 415 - DNA, 36 bp, Artificial Sequence
  >BioblockX2_414
cctagtagtcacagactgacagactcactcagctaa

SEQ ID NO: 416 - DNA, 36 bp, Artificial Sequence
  >BioblockX2_415
ctaagtagacactgagtgacactgagactcagggga

SEQ ID NO: 417 - DNA, 36 bp, Artificial Sequence
  >BioblockX2_416
aatagtagtcacagtctgacacactgtgtcagtcaa

SEQ ID NO: 418 - DNA, 36 bp, Artificial Sequence
  >BioblockX2_417
tcaagtagagtgacactgacagtgagagtcagcttc

SEQ ID NO: 419 - DNA, 36 bp, Artificial Sequence
  >BioblockX2_418
cttcgtagtcactgtctgacacagtctgtcagagta

SEQ ID NO: 420 - DNA, 36 bp, Artificial Sequence
  >BioblockX2_419
agtagtagtgtgtctgtgactctgacagtcagactg

SEQ ID NO: 421 - DNA, 36 bp, Artificial Sequence
  >BioblockX2_420
actggtagtcacagagtgacacactgactcagcaca

SEQ ID NO: 422 - DNA, 36 bp, Artificial Sequence
  >BioblockX2_421
cacagtagtcagactctgacacacactctcagccag

SEQ ID NO: 423 - DNA, 36 bp, Artificial Sequence
  >BioblockX2_422
ccaggtagacacagagtgacagactgtgtcagcaaa

SEQ ID NO: 424 - DNA, 36 bp, Artificial Sequence
  >BioblockX2_423
caaagtagagtctcagtgacagtgtctctcaggacc

SEQ ID NO: 425 - DNA, 36 bp, Artificial Sequence
  >BioblockX2_424
gaccgtagacactgactgacagacactgtcagactc

SEQ ID NO: 426 - DNA, 36 bp, Artificial Sequence
  >BioblockX2_425
actcgtagagagagtctgacacagtcactcagccac

SEQ ID NO: 427 - DNA, 36 bp, Artificial Sequence
  >BioblockX2_426
ccacgtagtgtgtcagtgacacacactgtcaggaac

SEQ ID NO: 428 - DNA, 36 bp, Artificial Sequence
  >BioblockX2_427
gaacgtagtcactgtctgactgtcactgtcaggcac

SEQ ID NO: 429 - DNA, 36 bp, Artificial Sequence
  >BioblockX2_428
gcacgtagtctctcagtgactctgagtgtcagcggc

SEQ ID NO: 430 - DNA, 36 bp, Artificial Sequence
  >BioblockX2_429
cggcgtagagtgagtctgacagtgacactcagcgta

SEQ ID NO: 431 - DNA, 36 bp, Artificial Sequence
  >BioblockX2_430
cgtagtagtcactctctgacacactgtgtcaggtaa

SEQ ID NO: 432 - DNA, 36 bp, Artificial Sequence
  >BioblockX2_431
gtaagtagacacagagtgactgtcacagtcagcaac

SEQ ID NO: 433 - DNA, 36 bp, Artificial Sequence
  >BioblockX2_432
caacgtagagacagtgtgacactgagtgtcaggcta

SEQ ID NO: 434 - DNA, 36 bp, Artificial Sequence
  >BioblockX2_433
gctagtagtcagtgagtgacacacactgtcagccga

SEQ ID NO: 435 - DNA, 36 bp, Artificial Sequence
  >BioblockX2_434
ccgagtagacagtcagtgacagagagactcagacga

SEQ ID NO: 436 - DNA, 36 bp, Artificial Sequence
  >BioblockX2_435
acgagtagagacagagtgacagtgtctgtcagagaa

SEQ ID NO: 437 - DNA, 36 bp, Artificial Sequence
  >BioblockX2_436
agaagtagactgtgactgactgtctgactcagtaaa

SEQ ID NO: 438 - DNA, 36 bp, Artificial Sequence
  >BioblockX2_437
taaagtagtgtgacagtgacacagtcactcagagcg

SEQ ID NO: 439 - DNA, 36 bp, Artificial Sequence
  >BioblockX2_438
agcggtagagagtcactgacactgacagtcagacct

SEQ ID NO: 440 - DNA, 36 bp, Artificial Sequence
  >BioblockX2_439
acctgtagtgagacactgactcagactgtcagaaca

SEQ ID NO: 441 - DNA, 36 bp, Artificial Sequence
  >BioblockX2_440
aacagtagtgacacactgacagtcactgtcagggca

SEQ ID NO: 442 - DNA, 36 bp, Artificial Sequence
  >BioblockX2_441
ggcagtagagtctctctgactcactgtctcagacgc

SEQ ID NO: 443 - DNA, 36 bp, Artificial Sequence
  >BioblockX2_442
acgcgtagacagacagtgacagagtgagtcagaatc

SEQ ID NO: 444 - DNA, 36 bp, Artificial Sequence
  >BioblockX2_443
aatcgtagacactgtctgacagacacagtcagcgag

SEQ ID NO: 445 - DNA, 36 bp, Artificial Sequence
  >BioblockX2_444
cgaggtagactgagagtgactctcacactcagtcca

SEQ ID NO: 446 - DNA, 36 bp, Artificial Sequence
  >BioblockX2_445
tccagtagacagtcactgacacactgagtcagccta

SEQ ID NO: 447 - DNA, 36 bp, Artificial Sequence
  >BioblockX2_446
cctagtagactcactctgactcagtgagtcagctaa

SEQ ID NO: 448 - DNA, 36 bp, Artificial Sequence
  >BioblockX2_447
ctaagtagtctgactgtgacactcagagtcagggga

SEQ ID NO: 449 - DNA, 36 bp, Artificial Sequence
  >BioblockX2_448
aatagtagacacagtgtgactgagagactcagtcaa

SEQ ID NO: 450 - DNA, 36 bp, Artificial Sequence
  >BioblockX2_449
tcaagtagagactgagtgactcagtctctcagcttc

SEQ ID NO: 451 - DNA, 36 bp, Artificial Sequence
  >BioblockX2_450
cttcgtagagactctgtgactctgtgtctcagagta

SEQ ID NO: 452 - DNA, 36 bp, Artificial Sequence
  >BioblockX2_451
agtagtagactgtcactgacactgacagtcagactg

SEQ ID NO: 453 - DNA, 36 bp, Artificial Sequence
  >BioblockX2_452
actggtagacacagagtgacacacagagtcagcaca

SEQ ID NO: 454 - DNA, 36 bp, Artificial Sequence
  >BioblockX2_453
cacagtagtcactctctgactcagagtgtcagccag

SEQ ID NO: 455 - DNA, 36 bp, Artificial Sequence
  >BioblockX2_454
ccaggtagagtgtcagtgactgtctgagtcagcaaa

SEQ ID NO: 456 - DNA, 36 bp, Artificial Sequence
  >BioblockX2_455
caaagtagagtgagagtgactctcagactcaggacc

SEQ ID NO: 457 - DNA, 36 bp, Artificial Sequence
  >BioblockX2_456
gaccgtagtcagacagtgacagtgagtgtcagactc

SEQ ID NO: 458 - DNA, 36 bp, Artificial Sequence
  >BioblockX2_457
actcgtagtctgactgtgacactgactctcagccac

SEQ ID NO: 459 - DNA, 36 bp, Artificial Sequence
  >BioblockX2_458
ccacgtagagtctcactgacagagagtctcaggaac

SEQ ID NO: 460 - DNA, 36 bp, Artificial Sequence
  >BioblockX2_459
gaacgtagagtgagtctgacacacagactcaggcac

SEQ ID NO: 461 - DNA, 36 bp, Artificial Sequence
  >BioblockX2_460
gcacgtagagtgacagtgactctctgagtcagcggc

SEQ ID NO: 462 - DNA, 36 bp, Artificial Sequence
  >BioblockX2_461
cggcgtagactctgagtgacagactctctcagcgta

SEQ ID NO: 463 - DNA, 36 bp, Artificial Sequence
  >BioblockX2_462
cgtagtagacagactgtgactcagtcagtcaggtaa

SEQ ID NO: 464 - DNA, 36 bp, Artificial Sequence
  >BioblockX2_463
gtaagtagtctcactctgactctctgtctcagcaac

SEQ ID NO: 465 - DNA, 36 bp, Artificial Sequence
  >BioblockX2_464
caacgtagacacagtctgactgactctgtcaggcta

SEQ ID NO: 466 - DNA, 36 bp, Artificial Sequence
  >BioblockX2_465
gctagtagtcactgtctgacacacacactcagccga

SEQ ID NO: 467 - DNA, 36 bp, Artificial Sequence
  >BioblockX2_466
ccgagtagtgtctgtctgacacagtcactcagacga

SEQ ID NO: 468 - DNA, 36 bp, Artificial Sequence
  >BioblockX2_467
acgagtagacagagagtgactctgtgtgtcagagaa

SEQ ID NO: 469 - DNA, 36 bp, Artificial Sequence
  >BioblockX2_468
agaagtagagagtctgtgacagtgtgagtcagtaaa

SEQ ID NO: 470 - DNA, 36 bp, Artificial Sequence
  >BioblockX2_469
taaagtagagtcacagtgactcactgactcagagcg

SEQ ID NO: 471 - DNA, 36 bp, Artificial Sequence
  >BioblockX2_470
agcggtagactgtcagtgactgtcagagtcagacct

SEQ ID NO: 472 - DNA, 36 bp, Artificial Sequence
  >BioblockX2_471
acctgtagacagagagtgactctgagactcagaaca

SEQ ID NO: 473 - DNA, 36 bp, Artificial Sequence
  >BioblockX2_472
aacagtagtcacacagtgactcagacagtcagggca

SEQ ID NO: 474 - DNA, 36 bp, Artificial Sequence
  >BioblockX2_473
ggcagtagtgagtgtctgacacactgactcagacgc

SEQ ID NO: 475 - DNA, 36 bp, Artificial Sequence
  >BioblockX2_474
acgcgtagtcagtcactgacactgactgtcagaatc

SEQ ID NO: 476 - DNA, 36 bp, Artificial Sequence
  >BioblockX2_475
aatcgtagactctgagtgactgtctgagtcagcgag

SEQ ID NO: 477 - DNA, 36 bp, Artificial Sequence
  >BioblockX2_476
cgaggtagactctgactgacacagactgtcagtcca

SEQ ID NO: 478 - DNA, 36 bp, Artificial Sequence
  >BioblockX2_477
tccagtagtgtgtgagtgacagagtctgtcagccta

SEQ ID NO: 479 - DNA, 36 bp, Artificial Sequence
  >BioblockX2_478
cctagtagagactgtctgactgacagtgtcagctaa

SEQ ID NO: 480 - DNA, 36 bp, Artificial Sequence
  >BioblockX2_479
ctaagtagtgactcactgacactcagtctcagggga

SEQ ID NO: 481 - DNA, 36 bp, Artificial Sequence
  >BioblockX2_480
aatagtagagagacactgacagactgtgtcagtcaa

SEQ ID NO: 482 - DNA, 36 bp, Artificial Sequence
  >BioblockX2_481
tcaagtagtgagtgtgtgacacagtgagtcagcttc

SEQ ID NO: 483 - DNA, 36 bp, Artificial Sequence
  >BioblockX2_482
cttcgtagactgtcactgacagtctgagtcagagta

SEQ ID NO: 484 - DNA, 36 bp, Artificial Sequence
  >BioblockX2_483
agtagtagtgtctgagtgacactgagagtcagactg

SEQ ID NO: 485 - DNA, 36 bp, Artificial Sequence
  >BioblockX2_484
actggtagacactgagtgacagtcactctcagcaca

SEQ ID NO: 486 - DNA, 36 bp, Artificial Sequence
  >BioblockX2_485
cacagtagtcagtgtgtgactcactcactcagccag

SEQ ID NO: 487 - DNA, 36 bp, Artificial Sequence
  >BioblockX2_486
ccaggtagtgagactctgacagtcagtctcagcaaa

SEQ ID NO: 488 - DNA, 36 bp, Artificial Sequence
  >BioblockX2_487
caaagtagtctgtgagtgactctgagtgtcaggacc

SEQ ID NO: 489 - DNA, 36 bp, Artificial Sequence
  >BioblockX2_488
gaccgtagacactgtctgacagagagtctcagactc

SEQ ID NO: 490 - DNA, 36 bp, Artificial Sequence
  >BioblockX2_489
actcgtagtctctctgtgactgtgtcactcagccac

SEQ ID NO: 491 - DNA, 36 bp, Artificial Sequence
  >BioblockX2_490
ccacgtagtcagacactgactcactgagtcaggaac

SEQ ID NO: 492 - DNA, 36 bp, Artificial Sequence
  >BioblockX2_491
gaacgtagagtgagagtgacacagtgactcaggcac

SEQ ID NO: 493 - DNA, 36 bp, Artificial Sequence
  >BioblockX2_492
gcacgtagacagagactgacagactgagtcagcggc

SEQ ID NO: 494 - DNA, 36 bp, Artificial Sequence
  >BioblockX2_493
cggcgtagagtgactgtgactcactcagtcagcgta

SEQ ID NO: 495 - DNA, 36 bp, Artificial Sequence
  >BioblockX2_494
cgtagtagtgacactctgacacagtcagtcaggtaa

SEQ ID NO: 496 - DNA, 36 bp, Artificial Sequence
  >BioblockX2_495
gtaagtagacagagagtgacagagtctgtcagcaac

SEQ ID NO: 497 - DNA, 36 bp, Artificial Sequence
  >BioblockX2_496
caacgtagtgtctcagtgactgtctcagtcaggcta

SEQ ID NO: 498 - DNA, 36 bp, Artificial Sequence
  >BioblockX2_497
gctagtagtctctctctgacacagtgagtcagccga

SEQ ID NO: 499 - DNA, 36 bp, Artificial Sequence
  >BioblockX2_498
ccgagtagagactctctgactgtcacactcagacga

SEQ ID NO: 500 - DNA, 36 bp, Artificial Sequence
  >BioblockX2_499
acgagtagacactcactgactgtcacagtcagagaa

SEQ ID NO: 501 - DNA, 36 bp, Artificial Sequence
  >BioblockX2_500
agaagtagtgacacagtgacagtgagactcagtaaa

SEQ ID NO: 502 - DNA, 36 bp, Artificial Sequence
  >BioblockX2_501
taaagtagtcacagtctgactctctgagtcagagcg

SEQ ID NO: 503 - DNA, 36 bp, Artificial Sequence
  >BioblockX2_502
agcggtagtgtgtcactgacagtcactctcagacct

SEQ ID NO: 504 - DNA, 36 bp, Artificial Sequence
  >BioblockX2_503
acctgtagtctgagtgtgacagagactctcagaaca

SEQ ID NO: 505 - DNA, 36 bp, Artificial Sequence
  >BioblockX2_504
aacagtagtgagagtgtgacactgtgtgtcagggca

SEQ ID NO: 506 - DNA, 36 bp, Artificial Sequence
  >BioblockX2_505
ggcagtagtcacagtctgactgtgtcactcagacgc

SEQ ID NO: 507 - DNA, 36 bp, Artificial Sequence
  >BioblockX2_506
acgcgtagtcacagagtgactcagtctctcagaatc

SEQ ID NO: 508 - DNA, 36 bp, Artificial Sequence
  >BioblockX2_507
aatcgtagtcactgtgtgactctgagtctcagcgag

SEQ ID NO: 509 - DNA, 36 bp, Artificial Sequence
  >BioblockX2_508
cgaggtagagactgtctgactcacagactcagtcca

SEQ ID NO: 510 - DNA, 36 bp, Artificial Sequence
  >BioblockX2_509
tccagtagagagtgactgacactgagtctcagccta

SEQ ID NO: 511 - DNA, 36 bp, Artificial Sequence
  >BioblockX2_510
cctagtagacacactctgacacagagtctcagctaa

SEQ ID NO: 512 - DNA, 36 bp, Artificial Sequence
  >BioblockX2_511
ctaagtagtgacagtgtgactgtgacagtcagggga

SEQ ID NO: 513 - DNA, 36 bp, Artificial Sequence; identical to SEQ ID NO: 1
  >BioblockX2_512

SEQ ID NO: 514 - DNA, 36 bp, Artificial Sequence
  >BioblockX2_513
tcaagtagacacacactgacacacacagtcagcttc

SEQ ID NO: 515 - DNA, 36 bp, Artificial Sequence
  >BioblockX2_514
cttcgtagagtgagtctgactcagagtgtcagagta

SEQ ID NO: 516 - DNA, 36 bp, Artificial Sequence
  >BioblockX2_515
agtagtagtgacacactgacactgtcagtcagactg

SEQ ID NO: 517 - DNA, 36 bp, Artificial Sequence
  >BioblockX2_516
actggtagacacagactgactctcagtctcagcaca

SEQ ID NO: 518 - DNA, 36 bp, Artificial Sequence
  >BioblockX2_517
cacagtagagagtgactgactgacagtctcagccag

SEQ ID NO: 519 - DNA, 36 bp, Artificial Sequence
  >BioblockX2_518
ccaggtagagtgtgtctgacactgagactcagcaaa

SEQ ID NO: 520 - DNA, 36 bp, Artificial Sequence
  >BioblockX2_519
caaagtagtgacagactgactgtgtcagtcaggacc

SEQ ID NO: 521 - DNA, 36 bp, Artificial Sequence
  >BioblockX2_520
gaccgtagactgtctgtgacagtctcagtcagactc

SEQ ID NO: 522 - DNA, 36 bp, Artificial Sequence
  >BioblockX2_521
actcgtagtgtgagtctgacactcagactcagccac

SEQ ID NO: 523 - DNA, 36 bp, Artificial Sequence
  >BioblockX2_522
ccacgtagagactgtctgacagagtctctcaggaac

SEQ ID NO: 524 - DNA, 36 bp, Artificial Sequence
  >BioblockX2_523
gaacgtagacacacactgacacacacagtcaggcac

SEQ ID NO: 525 - DNA, 36 bp, Artificial Sequence
  >BioblockX2_524
gcacgtagacacagactgacacacagtctcagcggc

SEQ ID NO: 526 - DNA, 36 bp, Artificial Sequence
  >BioblockX2_525
cggcgtagacacacactgacacacacagtcagcgta

SEQ ID NO: 527 - DNA, 36 bp, Artificial Sequence
  >BioblockX2_526
cgtagtagacactcagtgactcacactgtcaggtaa

SEQ ID NO: 528 - DNA, 36 bp, Artificial Sequence
  >BioblockX2_527
gtaagtagtgtctgtgtgacacacacactcagcaac

SEQ ID NO: 529 - DNA, 36 bp, Artificial Sequence
  >BioblockX2_528
caacgtagacacagtgtgacacactctgtcaggcta

SEQ ID NO: 530 - DNA, 36 bp, Artificial Sequence
  >BioblockX2_529
gctagtagacacacagtgacacacacactcagccga

SEQ ID NO: 531 - DNA, 36 bp, Artificial Sequence
  >BioblockX2_530
ccgagtagacacacagtgacactcacagtcagacga

SEQ ID NO: 532 - DNA, 36 bp, Artificial Sequence
  >BioblockX2_531
acgagtagactcacagtgacacacacagtcagagaa

SEQ ID NO: 533 - DNA, 36 bp, Artificial Sequence
  >BioblockX2_532
agaagtagacacacactgacacactgactcagtaaa

SEQ ID NO: 534 - DNA, 36 bp, Artificial Sequence
  >BioblockX2_533
taaagtagtcactcagtgacactgagtctcagagcg

SEQ ID NO: 535 - DNA, 36 bp, Artificial Sequence
  >BioblockX2_534
agcggtagacacacactgacacactcactcagacct

SEQ ID NO: 536 - DNA, 36 bp, Artificial Sequence
  >BioblockX2_535
acctgtagacactctctgacacacacagtcagaaca

SEQ ID NO: 537 - DNA, 36 bp, Artificial Sequence
  >BioblockX2_536
aacagtagacactctctgacagactgactcagggca

SEQ ID NO: 538 - DNA, 36 bp, Artificial Sequence
  >BioblockX2_537
ggcagtagacacactctgacacagtgtgtcagacgc

SEQ ID NO: 539 - DNA, 36 bp, Artificial Sequence
  >BioblockX2_538
acgcgtagacacacactgacacacacactcagaatc

SEQ ID NO: 540 - DNA, 36 bp, Artificial Sequence
  >BioblockX2_539
aatcgtagacacagagtgacacacacagtcagcgag

SEQ ID NO: 541 - DNA, 36 bp, Artificial Sequence
  >BioblockX2_540
cgaggtagacagtcagtgacacactctctcagtcca

SEQ ID NO: 542 - DNA, 36 bp, Artificial Sequence
  >BioblockX2_541
tccagtagacacacactgacacacacactcagccta

SEQ ID NO: 543 - DNA, 36 bp, Artificial Sequence
  >BioblockX2_542
cctagtagacacacactgacacacacactcagctaa

SEQ ID NO: 544 - DNA, 36 bp, Artificial Sequence
  >BioblockX2_543
ctaagtagacacacactgacacacacactcagggga

SEQ ID NO: 545 - DNA, 36 bp, Artificial Sequence; identical to SEQ ID NO: 1
  >BioblockX2_544

SEQ ID NO: 546 - DNA, 36 bp, Artificial Sequence; identical to SEQ ID NO: 2
  >BioblockX2_545

SEQ ID NO: 547 - DNA, 36 bp, Artificial Sequence
  >BioblockX2_546
cttcgtagacagacagtgacacagtcagtcagagta

SEQ ID NO: 548 - DNA, 36 bp, Artificial Sequence
  >BioblockX2_547
agtagtagacacacactgacagactgactcagactg

SEQ ID NO: 549 - DNA, 36 bp, Artificial Sequence
  >BioblockX2_548
actggtagacacacactgacagtctcagtcagcaca

SEQ ID NO: 550 - DNA, 36 bp, Artificial Sequence
  >BioblockX2_549
cacagtagacacacactgacagtcactctcagccag

SEQ ID NO: 551 - DNA, 36 bp, Artificial Sequence
  >BioblockX2_550
ccaggtagacacacactgacagtcagagtcagcaaa

SEQ ID NO: 552 - DNA, 36 bp, Artificial Sequence
  >BioblockX2_551
caaagtagacacacactgacagtgactctcaggacc

SEQ ID NO: 553 - DNA, 36 bp, Artificial Sequence
  >BioblockX2_552
gaccgtagacacacactgacagtgagactcagactc

SEQ ID NO: 554 - DNA, 36 bp, Artificial Sequence
  >BioblockX2_553
actcgtagacacacactgactgtctcagtcagccac

SEQ ID NO: 555 - DNA, 36 bp, Artificial Sequence
  >BioblockX2_554
ccacgtagacacacactgacactctgtctcaggaac

SEQ ID NO: 556 - DNA, 36 bp, Artificial Sequence
  >BioblockX2_555
gaacgtagacacacactgacagtgagactcaggcac

SEQ ID NO: 557 - DNA, 36 bp, Artificial Sequence
  >BioblockX2_556
gcacgtagacacacactgacagtgtcactcagcggc

SEQ ID NO: 558 - DNA, 36 bp, Artificial Sequence
  >BioblockX2_557
cggcgtagacacacactgacagtgagactcagcgta

SEQ ID NO: 559 - DNA, 36 bp, Artificial Sequence; identical to SEQ ID NO: 15
  >BioblockX2_558

SEQ ID NO: 560 - DNA, 36 bp, Artificial Sequence
  >BioblockX2_559
gtaagtagacacacactgacacagtcagtcagcaac

SEQ ID NO: 561 - DNA, 36 bp, Artificial Sequence
  >BioblockX2_560
caacgtagacacactctgacacacacactcaggcta

SEQ ID NO: 562 - DNA, 36 bp, Artificial Sequence
  >BioblockX2_561
gctagtagacacacactgacacagagactcagccga

SEQ ID NO: 563 - DNA, 36 bp, Artificial Sequence
  >BioblockX2_562
ccgagtagacactctctgacacacacagtcagacga

SEQ ID NO: 564 - DNA, 36 bp, Artificial Sequence
  >BioblockX2_563
acgagtagacacacactgacacacacactcagagaa

SEQ ID NO: 565 - DNA, 36 bp, Artificial Sequence
  >BioblockX2_564
agaagtagtgtgagtgtgactgtcagtctcagtaaa

SEQ ID NO: 566 - DNA, 36 bp, Artificial Sequence
  >BioblockX2_565
taaagtagtctgactgtgacagtcactgtcagagcg

SEQ ID NO: 567 - DNA, 36 bp, Artificial Sequence
  >BioblockX2_566
agcggtagactgtgtgtgactgagtcactcagacct

SEQ ID NO: 568 - DNA, 36 bp, Artificial Sequence
  >BioblockX2_567
acctgtagacacacagtgacacagagagtcagaaca

SEQ ID NO: 569 - DNA, 36 bp, Artificial Sequence
  >BioblockX2_568
aacagtagacacagtctgacacacacagtcagggca

SEQ ID NO: 570 - DNA, 36 bp, Artificial Sequence
  >BioblockX2_569
ggcagtagacacacactgacactcacactcagacgc

SEQ ID NO: 571 - DNA, 36 bp, Artificial Sequence
  >BioblockX2_570
acgcgtagtcacacactgactctgagactcagaatc

SEQ ID NO: 572 - DNA, 36 bp, Artificial Sequence
  >BioblockX2_571
aatcgtagtcacacagtgacacacacactcagcgag

SEQ ID NO: 573 - DNA, 36 bp, Artificial Sequence
  >BioblockX2_572
cgaggtagacacacactgaccagtctgttcagtcca

SEQ ID NO: 574 - DNA, 1036 bp, Artificial Sequence
  >BioblockX64_0
aataaatagtagacacacactgacacacacactcagtcaagtagacacacactgacacacacactcagcttcgtagactgagtgtgacagtgtctctcagagtagtagtctgtgactgactctctgtgtcagactggtagactcagtgtgacacagtgactcagcacagtagacacacactgacacacagactcagccaggtagtgagagactgactgagtgtgtcagcaaagtagtgtgtcagtgactctgtgactcaggaccgtagtgtctgtgtgacacacactgtcagactcgtagacacacactgacacacacactcagccacgtagacacacactgacacacacactcaggaacgtagacacacactgacacacacactcaggcacgtagagtcactctgacacacacactcagcggcgtagacacacactgacacacacactcagcgtagtagacacacactgacacacacactcaggtaagtagacacacactgacacacacactcagcaacgtagtgagtcactgacagactgtctcaggctagtagtcagagactgactcagtgtgtcagccgagtagtgtcacactgacacactcagtcagacgagtagactgagagtgacacacactgtcagagaagtagtgtcagtgtgacagagtgagtcagtaaagtagacacacactgacactcagtctcagagcggtagacagtctctgacagactcactcagacctgtagagacagtctgacagtgacagtcagaacagtagtgtctctctgacactcacagtcagggcagtagactgtgtctgacagactcagtcagacgcgtagtctctgtgtgactgtctgtgtcagaatcgtagtgtcacagtgactctgtgtctcagcgaggtagagtcacactgactgagacagtcagtccagtagacagactctgacagtgactgtcagcctagtagagtgtgactgactcacagtctcagctaagtagacacagactgacagacacactcaggggaaagg

SEQ ID NO: 575 - DNA, 1036 bp, Artificial Sequence
  >BioblockX64_1
aaggaatagtagacacactctgactgtctgtgtcagtcaagtagtgagagtgtgactctcactgtcagcttcgtagtcagtcactgactcactctctcagagtagtagacacactgtgactgagtgtctcagactggtagtgtgagtctgacacactgagtcagcacagtagagagtgactgactctcagagtcagccaggtagtcagtcagtgactgagacactcagcaaagtagacagtgagtgacagtgactgtcaggaccgtagactgtgtgtgacacagacactcagactcgtagtgtgtgactgacactcacactcagccacgtagtctgactgtgacagtcagtctcaggaacgtagacacagactgacactctctctcaggcacgtagagacacagtgactctcacagtcagcggcgtagtgtctgactgacacacagactcagcgtagtagacacagtgtgactctctctgtcaggtaagtagtcagtctgtgactcacagtctcagcaacgtagagtcagtctgactgtctgactcaggctagtagtctcactgtgactctctctgtcagccgagtagactgagagtgactctcacagtcagacgagtagtgagtctctgacacagtctctcagagaagtagacacagactgacacactgtgtcagtaaagtagactctctgtgacagactctgtcagagcggtagtcagtcactgacactgagagtcagacctgtagtgacagactgactgagacagtcagaacagtagtgtcagtctgacagtcactctcagggcagtagacagacagtgactgtcagtctcagacgcgtagtctctctgtgactgtctcagtcagaatcgtagagtgtgagtgacagagtctctcagcgaggtagactctgtgtgactctgtctctcagtccagtagactgagagtgactcagtgtctcagcctagtagacactgagtgactctcagtctcagctaagtagactctgagtgacagtgagtgtcaggggaaaac

SEQ ID NO: 576 - DNA, 1036 bp, Artificial Sequence
  >BioblockX64_2
aaacaatagtagacagacagtgacacacactgtcagtcaagtagacagagactgactgtcactgtcagcttcgtagtgactctgtgacagagagtctcagagtagtagtcactctgtgacacagtgtgtcagactggtagtgtcagtgtgacactgtcagtcagcacagtagtgtgtcagtgactctgactctcagccaggtagactctctctgacagtgtgagtcagcaaagtagactcagagtgacagacacactcaggaccgtagtgacagtgtgacactgtcactcagactcgtagactctgtgtgacagtgagagtcagccacgtagtcagtgagtgactcagtgtgtcaggaacgtagagtcacagtgacacagtgagtcaggcacgtagagtgtcactgactctgtcagtcagcggcgtagtcagtctgtgactctctgtctcagcgtagtagactgagtgtgacagacacactcaggtaagtagtgtgagagtgactcacagtctcagcaacgtagactgtgagtgactgacactgtcaggctagtagagagtcagtgactcagagtctcagccgagtagtgtcacactgactgactctctcagacgagtagagtctctctgactcagagtgtcagagaagtagactgagtgtgactctcactctcagtaaagtagtgtcagagtgacagtgacagtcagagcggtagtgtgactgtgacagtctcagtcagacctgtagtgtgtctctgactgtcagactcagaacagtagactctctgtgactcagacagtcagggcagtagtgtgagtgtgacactcagtgtcagacgcgtagtctgtgagtgactgagacagtcagaatcgtagtgagagtctgacagagactgtcagcgaggtagagagtgtgtgactctcagactcagtccagtagagagagagtgacagagagtgtcagcctagtagacacagtctgacagagtctctcagctaagtagtcagagtctgactgactgagtcaggggataaa

SEQ ID NO: 577 - DNA, 1036 bp, Artificial Sequence
  >BioblockX64_3
taaaaatagtagagtctgtgtgactgtgagtctcagtcaagtagacacacagtgacactctcactcagcttcgtagagacacagtgactgtgtgtctcagagtagtagtgactctctgactctgtcagtcagactggtagactgtctctgactgtctgtctcagcacagtagtgtctctctgacagtgtcagtcagccaggtagactgacagtgactctctgagtcagcaaagtagtgagactctgacagtgacactcaggaccgtagagtcactgtgactcagtctgtcagactcgtagtcactgtctgacagtgtgactcagccacgtagtgtgacagtgacagtgtgagtcaggaacgtagtcagtgactgactgagagagtcaggcacgtagagacagagtgacacagacactcagcggcgtagtgagagtgtgacactgagactcagcgtagtagacagtcactgactgagtctgtcaggtaagtagactgtcagtgactgactctgtcagcaacgtagtctctctgtgactgagacactcaggctagtagtcagagactgacagtgtgactcagccgagtagagacacagtgacagtgagtctcagacgagtagtgacagagtgactctgtcactcagagaagtagtctctcactgactctgactctcagtaaagtagtgagagtgtgactgtgtcactcagagcggtagtcagacagtgactgtgtgtgtcagacctgtagagagactctgacagtgagagtcagaacagtagagtctgtctgacactctcagtcagggcagtagtctcagactgactgtctgagtcagacgcgtagactcagtgtgacagacactctcagaatcgtagtcacagtctgactgtgtcactcagcgaggtagacactgagtgacactctgagtcagtccagtagtcagagtctgactgtgacactcagcctagtagtgagacagtgacagtgtcactcagctaagtagacagagtgtgactgagtctgtcaggggaacga

SEQ ID NO: 578 - DNA, 1036 bp, Artificial Sequence
  >BioblockX64_4
acgaaatagtagagtgacagtgactcagagactcagtcaagtagactctgagtgacactgtgtgtcagcttcgtagtctgacactgactgtgacagtcagagtagtagtcagacagtgacagactgactcagactggtagagtcagactgacactcagactcagcacagtagagacagagtgactgtctcactcagccaggtagtgacagtgtgacagtgacagtcagcaaagtagtgagagactgactctcactgtcaggaccgtagtgtgtcagtgactcacactgtcagactcgtagtgtctctgtgacacactgtgtcagccacgtagagtctcagtgactctcagactcaggaacgtagagacactctgacagtgtgtctcaggcacgtagagagagactgactctctcagtcagcggcgtagagtcacagtgactgtgagagtcagcgtagtagtgtcagtgtgactctgactctcaggtaagtagtgtgtgtctgacagtcacagtcagcaacgtagtcacactctgacagagacactcaggctagtagacactgactgacactcactctcagccgagtagagtgagagtgacagacagtgtcagacgagtagtgagactctgactgagtgagtcagagaagtagtctgagtgtgacagtgtgagtcagtaaagtagtctgagagtgacacagtgtctcagagcggtagagagactgtgactctcagtgtcagacctgtagactctgagtgactctgtgtctcagaacagtagtgagactctgactcagacactcagggcagtagagtgagtctgacacactcagtcagacgcgtagtgtgagtctgacagtctcactcagaatcgtagagactgagtgactgagactctcagcgaggtagtgacagtgtgactctgtctctcagtccagtagagtgagtctgacagagagactcagcctagtagactctctgtgacacagagactcagctaagtagacacagactgacagagacagtcaggggaactg

SEQ ID NO: 579 - DNA, 1036 bp, Artificial Sequence
  >BioblockX64_5
actgaatagtagagtctgtctgactgtctgagtcagtcaagtagagagtgagtgacagtcacagtcagcttcgtagtgagagagtgactgtcactgtcagagtagtagtctgtgagtgacactcacactcagactggtagtgagagactgactgtgtcagtcagcacagtagtgactgagtgacactgacagtcagccaggtagagagtgagtgactgagagactcagcaaagtagacagagagtgacacacagactcaggaccgtagtgagtcactgactgtgagactcagactcgtagtctgagtctgactgactctgtcagccacgtagactcactctgacacactgagtcaggaacgtagtcacacagtgactctctcactcaggcacgtagagactgactgacagagacagtcagcggcgtagtgagagtctgactctcagtctcagcgtagtagacagagagtgacagactctgtcaggtaagtagtgactgagtgacactcactctcagcaacgtagtcactgactgactgtctcactcaggctagtagtgtgacactgactcacacactcagccgagtagtctgtgtctgactcactgagtcagacgagtagtctctgagtgactctgagactcagagaagtagacacagtgtgactcactgactcagtaaagtagagacagactgacactgtgactcagagcggtagacacactctgactctctgactcagacctgtagacacagtctgacacacacactcagaacagtagacagagactgactcacacagtcagggcagtagacagagagtgacacagtcactcagacgcgtagagactgtctgactcagactgtcagaatcgtagtcacacactgacactgacagtcagcgaggtagtgagtgtctgacacactcagtcagtccagtagactcactctgactgagagagtcagcctagtagtcacacagtgactctgtcagtcagctaagtagacagagtgtgactgtcagtctcaggggaagcg

SEQ ID NO: 580 - DNA, 1036 bp, Artificial Sequence
  >BioblockX64_6
agcgaatagtagtctctcagtgactctgagagtcagtcaagtagtcagagtctgacactgtgtgtcagcttcgtagagtctcactgacacagtgagtcagagtagtagtgagagagtgacagagagagtcagactggtagtgacagactgacagactgagtcagcacagtagtcagacactgacagtgagagtcagccaggtagtgtcagtgtgacagacacactcagcaaagtagagtctctctgacactgtgagtcaggaccgtagtctcactctgactcagactctcagactcgtagtgactgagtgactgactcagtcagccacgtagtcacagtgtgactgagagagtcaggaacgtagtgtcagtctgactgtgtctgtcaggcacgtagtcagagtctgacagagtgagtcagcggcgtagtcagagtctgacactctgagtcagcgtagtagtctgagactgacacactcactcaggtaagtagtgtgagactgactgtcacagtcagcaacgtagtctgactgtgacacagagtctcaggctagtagacagactgtgactgactgtctcagccgagtagtctcagtctgactgagtcactcagacgagtagtgagacactgacactgtgactcagagaagtagtctgtctgtgacactcacactcagtaaagtagtgtcagtgtgacagagtgagtcagagcggtagtcacacactgactcactctgtcagacctgtagtgactctctgactgactgtctcagaacagtagacagtgtgtgacacacacagtcagggcagtagtctgagagtgactgtgtcagtcagacgcgtagtctgagactgacactgtcactcagaatcgtagactgtgagtgacagtgagactcagcgaggtagtgtcacactgacactcactctcagtccagtagtgtgtgagtgactctcagtctcagcctagtagacacacactgacacagtgtctcagctaagtagtgagagtctgactcactctgtcaggggagcta

SEQ ID NO: 581 - DNA, 1036 bp, Artificial Sequence
  >BioblockX64_7
gctaaatagtagactgtgtctgactgtgtgagtcagtcaagtagactgacagtgactcacagactcagcttcgtagtgagtgagtgactgtgagtgtcagagtagtagtctcagactgactgtgtgagtcagactggtagactcacactgacactgactctcagcacagtagacacacagtgactcagtcactcagccaggtagagtgtcactgactcactctctcagcaaagtagtcactgactgacactcagtctcaggaccgtagtctgtgagtgacactcacactcagactcgtagagagactgtgacacagtgagtcagccacgtagtctcacagtgacactgtgtctcaggaacgtagagtctgagtgacagtctgactcaggcacgtagtcagtctgtgactctctctgtcagcggcgtagagtgtctctgactgagtgagtcagcgtagtagtcagacagtgacactgagagtcaggtaagtagtgtgagactgacagtcagagtcagcaacgtagtgactcagtgacagagtctctcaggctagtagtctgtcactgactgtctcagtcagccgagtagtgtgtgagtgacagtctctctcagacgagtagagactgtctgactgagactgtcagagaagtagtgactcagtgactgacactctcagtaaagtagacagactgtgacactgtcactcagagcggtagtcactcagtgacacactctgtcagacctgtagtgtctgtgtgactgtctctctcagaacagtagacactgtctgactgacagtgtcagggcagtagtctctctgtgacacagtctgtcagacgcgtagtcacagagtgactgactctctcagaatcgtagactgactgtgacacagtctgtcagcgaggtagtgagacactgacagtgagtgtcagtccagtagagtctgagtgactctcagactcagcctagtagtctgtgtgtgactgtgagtgtcagctaagtagtcactgtctgacagactgtctcaggggaggca

SEQ ID NO: 582 - DNA, 1036 bp, Artificial Sequence
  >BioblockX64_8
ggcaaatagtagagtctctctgacagtcagactcagtcaagtagtcacacagtgactcactctctcagcttcgtagagtctgtgtgacagagtgagtcagagtagtagtgactctgtgacacactgtctcagactggtagagtgtctctgactgactgagtcagcacagtagtgtctctgtgacactcacagtcagccaggtagtgagtctgtgactgactgtgtcagcaaagtagtctgagagtgacagtcacactcaggaccgtagtcagtctgtgacacacactctcagactcgtagtctctgagtgactgagtgtctcagccacgtagactcacactgacagtctgactcaggaacgtagactgacactgactcagtgagtcaggcacgtagtgactcactgactcacacactcagcggcgtagtctgtgtgtgactctgagtgtcagcgtagtagtgacactgtgactgtctctgtcaggtaagtagtcagacactgacactgtcactcagcaacgtagactcagtgtgacactgacagtcaggctagtagtgacagagtgactctctgagtcagccgagtagactcagagtgacagagacagtcagacgagtagacagacagtgactgagtgactcagagaagtagtgagtcactgacagtcagtgtcagtaaagtagtgactgactgactgtcacagtcagagcggtagacagagactgactgagtgactcagacctgtagagagacactgactgtcacagtcagaacagtagtcagacagtgacagagtcagtcagggcagtagtctcacagtgactctctctgtcagacgcgtagacagtcagtgactgtgactgtcagaatcgtagtgtctcagtgactcactcagtcagcgaggtagactgacagtgacactgtctctcagtccagtagtcagagagtgacagtcacagtcagcctagtagtgtcactctgactgtgtgtgtcagctaagtagtgagtcactgactgagagtctcaggggaacct

SEQ ID NO: 583 - DNA, 1036 bp, Artificial Sequence
  >BioblockX64_9
acctaatagtagtgagtctgtgacacactcagtcagtcaagtagactgtgtgtgacactgtgtgtcagcttcgtagtcactctctgacacacagtgtcagagtagtagagagacactgacagacactctcagactggtagtctgtgtgtgactgtctctgtcagcacagtagtctcagagtgacagtcactgtcagccaggtagtcagacagtgactgacagtctcagcaaagtagagtctgtgtgacactcactgtcaggaccgtagtgtgacagtgacacagtctctcagactcgtagagacactgtgacacagtctgtcagccacgtagagactcactgacactgtgagtcaggaacgtagacactgtctgactcagtcagtcaggcacgtagagagagtctgacagactgagtcagcggcgtagtctgtctgtgacactgtgtctcagcgtagtagacactgtctgacagtctgactcaggtaagtagagagacagtgacagtgagtgtcagcaacgtagagagactctgacagtgagagtcaggctagtagagtgagagtgacacacacagtcagccgagtagtgacagtctgacactctctgtcagacgagtagtgacacactgactgtgtctctcagagaagtagtgtgtgagtgacacactgtgtcagtaaagtagacagagtgtgacacactcactcagagcggtagtgtcagtgtgacagtctctgtcagacctgtagagactcagtgactctgtgactcagaacagtagtctgagtctgacagtctgactcagggcagtagacagactctgacagactcagtcagacgcgtagactgtgtctgactcagacagtcagaatcgtagactcacactgactctgactgtcagcgaggtagagtcagagtgacacagtcagtcagtccagtagtgacagagtgactgtctctctcagcctagtagagtgtgactgacacagtgactcagctaagtagagtcactgtgacagtgtgactcaggggacgta

SEQ ID NO: 584 - DNA, 1036 bp, Artificial Sequence
  >BioblockX64_10
cgtaaatagtagactcagtgtgacactgagagtcagtcaagtagagtgagtgtgactgtgagagtcagcttcgtagagacagagtgactgtgacactcagagtagtagagacagagtgactcagtgtgtcagactggtagactgacactgacacacagagtcagcacagtagagactgtgtgacacacacagtcagccaggtagtgactgagtgacacacacactcagcaaagtagagtgacagtgacacagacagtcaggaccgtagtctcagtgtgactctctctctcagactcgtagagagactctgactgtgtgagtcagccacgtagtgtgactgtgactgagtgtgtcaggaacgtagagagtcagtgacactctgtctcaggcacgtagtctgtctgtgacacagagactcagcggcgtagtgtgtgactgacagtcactctcagcgtagtagagactcagtgacacagtcagtcaggtaagtagagactgtgtgacactgagtctcagcaacgtagagagtctgtgactgtgtctgtcaggctagtagtcagtgtgtgactcacagtgtcagccgagtagacagagactgactcagtgtctcagacgagtagagtctctctgacacagagagtcagagaagtagacagtgactgacagagacagtcagtaaagtagagtgacactgacagtctgactcagagcggtagtgacagagtgactgacagagtcagacctgtagtgtgactctgactgactgactcagaacagtagtcagagtctgactcactgagtcagggcagtagactgagagtgacactctctgtcagacgcgtagtctcacagtgactgtctctgtcagaatcgtagtgtctgactgacactgtctgtcagcgaggtagtgacagactgactctgagagtcagtccagtagagagagactgacagagtcagtcagcctagtagtgtctgagtgactgagtctgtcagctaagtagactctgagtgacacagtgtctcaggggaaaca

SEQ ID NO: 585 - DNA, 1036 bp, Artificial Sequence
  >BioblockX64_11
aacaaatagtagtctcagagtgacagagagtgtcagtcaagtagtctgtgactgacagtgagtgtcagcttcgtagacagtcactgacagtcagtgtcagagtagtagacacactgtgacacactctgtcagactggtagactgtctgtgactcagtgagtcagcacagtagtgtgagtgtgactgactgtgtcagccaggtagagtgtcagtgactctctgagtcagcaaagtagactcagactgactgacagtgtcaggaccgtagagtcacagtgacactctcactcagactcgtagtcagtctgtgacacactctgtcagccacgtagtctgagagtgactcactgtctcaggaacgtagtgtgtcactgactgagacagtcaggcacgtagtgagtctgtgactgtctgtgtcagcggcgtagtcactcagtgactgtgtgagtcagcgtagtagacagtgagtgacactgactctcaggtaagtagtcactcactgactgacagactcagcaacgtagtctctcagtgactgagtcagtcaggctagtagacagtgactgactcagtgagtcagccgagtagtgagtgagtgactctctgtctcagacgagtagtgactctctgacagagacagtcagagaagtagtgtgtcactgactgtcactgtcagtaaagtagtcagacagtgacagtgactgtcagagcggtagtctcacactgactgtgtgactcagacctgtagagtgacagtgacagagacagtcagaacagtagtgtcagactgacagtctgtctcagggcagtagtgagagactgacagactctctcagacgcgtagagtgacagtgactgactgagtcagaatcgtagtcagtgagtgacacagtgtctcagcgaggtagtgacagtctgacacactcactcagtccagtagactgactgtgactgtgtcactcagcctagtagagtgagactgactgtgagtctcagctaagtagagacacactgacagacacactcaggggactac

SEQ ID NO: 586 - DNA, 1036 bp, Artificial Sequence
  >BioblockX64_12
ctacaatagtagtgtctgactgactgtgtgtctcagtcaagtagtcagagtgtgactctgagtctcagcttcgtagagacagtctgacagactcactcagagtagtagtgtgtctctgacacacactctcagactggtagtctgacagtgactgacactctcagcacagtagtgacacactgacagacagtgtcagccaggtagtctgagactgacagtgacagtcagcaaagtagagactcactgactcagactctcaggaccgtagacagagtgtgactcagtctgtcagactcgtagagtcagtctgacacagtctctcagccacgtagtgagactgtgactcacactgtcaggaacgtagtcagacactgactgtgtcactcaggcacgtagacagactctgactgtcactgtcagcggcgtagtgtgtctctgactgagtgtctcagcgtagtagtcagtctgtgacagtcagtgtcaggtaagtagtgactcagtgactgtgtctgtcagcaacgtagactctcactgacagtcagtgtcaggctagtagtcagagagtgactgacacagtcagccgagtagacacagagtgacagtgagtctcagacgagtagtcactcactgactcactcagtcagagaagtagtcactcactgactcagacagtcagtaaagtagacactctgtgacactgacactcagagcggtagtgacagtgtgacactcactgtcagacctgtagtgtcacagtgacacacacagtcagaacagtagacactctctgactgtctctgtcagggcagtagagtgtgactgactcactcactcagacgcgtagtgagagtgtgacagagagagtcagaatcgtagagtgacactgacactgactctcagcgaggtagagagtgtgtgactgacacactcagtccagtagtgtgtgtgtgactcagtgagtcagcctagtagtcacagactgacagactcactcagctaagtagacactgagtgacactgagactcaggggagaga

SEQ ID NO: 587 - DNA, 1036 bp, Artificial Sequence
  >BioblockX64_13
gagaaatagtagtcacagtctgacacactgtgtcagtcaagtagagtgacactgacagtgagagtcagcttcgtagtcactgtctgacacagtctgtcagagtagtagtgtgtctgtgactctgacagtcagactggtagtcacagagtgacacactgactcagcacagtagtcagactctgacacacactctcagccaggtagacacagagtgacagactgtgtcagcaaagtagagtctcagtgacagtgtctctcaggaccgtagacactgactgacagacactgtcagactcgtagagagagtctgacacagtcactcagccacgtagtgtgtcagtgacacacactgtcaggaacgtagtcactgtctgactgtcactgtcaggcacgtagtctctcagtgactctgagtgtcagcggcgtagagtgagtctgacagtgacactcagcgtagtagtcactctctgacacactgtgtcaggtaagtagacacagagtgactgtcacagtcagcaacgtagagacagtgtgacactgagtgtcaggctagtagtcagtgagtgacacacactgtcagccgagtagacagtcagtgacagagagactcagacgagtagagacagagtgacagtgtctgtcagagaagtagactgtgactgactgtctgactcagtaaagtagtgtgacagtgacacagtcactcagagcggtagagagtcactgacactgacagtcagacctgtagtgagacactgactcagactgtcagaacagtagtgacacactgacagtcactgtcagggcagtagagtctctctgactcactgtctcagacgcgtagacagacagtgacagagtgagtcagaatcgtagacactgtctgacagacacagtcagcgaggtagactgagagtgactctcacactcagtccagtagacagtcactgacacactgagtcagcctagtagactcactctgactcagtgagtcagctaagtagtctgactgtgacactcagagtcaggggaccag

SEQ ID NO: 588 - DNA, 1036 bp, Artificial Sequence
  >BioblockX64_14
ccagaatagtagacacagtgtgactgagagactcagtcaagtagagactgagtgactcagtctctcagcttcgtagagactctgtgactctgtgtctcagagtagtagactgtcactgacactgacagtcagactggtagacacagagtgacacacagagtcagcacagtagtcactctctgactcagagtgtcagccaggtagagtgtcagtgactgtctgagtcagcaaagtagagtgagagtgactctcagactcaggaccgtagtcagacagtgacagtgagtgtcagactcgtagtctgactgtgacactgactctcagccacgtagagtctcactgacagagagtctcaggaacgtagagtgagtctgacacacagactcaggcacgtagagtgacagtgactctctgagtcagcggcgtagactctgagtgacagactctctcagcgtagtagacagactgtgactcagtcagtcaggtaagtagtctcactctgactctctgtctcagcaacgtagacacagtctgactgactctgtcaggctagtagtcactgtctgacacacacactcagccgagtagtgtctgtctgacacagtcactcagacgagtagacagagagtgactctgtgtgtcagagaagtagagagtctgtgacagtgtgagtcagtaaagtagagtcacagtgactcactgactcagagcggtagactgtcagtgactgtcagagtcagacctgtagacagagagtgactctgagactcagaacagtagtcacacagtgactcagacagtcagggcagtagtgagtgtctgacacactgactcagacgcgtagtcagtcactgacactgactgtcagaatcgtagactctgagtgactgtctgagtcagcgaggtagactctgactgacacagactgtcagtccagtagtgtgtgagtgacagagtctgtcagcctagtagagactgtctgactgacagtgtcagctaagtagtgactcactgacactcagtctcaggggaagaa

SEQ ID NO: 589 - DNA, 1036 bp, Artificial Sequence
  >BioblockX64_15
agaaaatagtagagagacactgacagactgtgtcagtcaagtagtgagtgtgtgacacagtgagtcagcttcgtagactgtcactgacagtctgagtcagagtagtagtgtctgagtgacactgagagtcagactggtagacactgagtgacagtcactctcagcacagtagtcagtgtgtgactcactcactcagccaggtagtgagactctgacagtcagtctcagcaaagtagtctgtgagtgactctgagtgtcaggaccgtagacactgtctgacagagagtctcagactcgtagtctctctgtgactgtgtcactcagccacgtagtcagacactgactcactgagtcaggaacgtagagtgagagtgacacagtgactcaggcacgtagacagagactgacagactgagtcagcggcgtagagtgactgtgactcactcagtcagcgtagtagtgacactctgacacagtcagtcaggtaagtagacagagagtgacagagtctgtcagcaacgtagtgtctcagtgactgtctcagtcaggctagtagtctctctctgacacagtgagtcagccgagtagagactctctgactgtcacactcagacgagtagacactcactgactgtcacagtcagagaagtagtgacacagtgacagtgagactcagtaaagtagtcacagtctgactctctgagtcagagcggtagtgtgtcactgacagtcactctcagacctgtagtctgagtgtgacagagactctcagaacagtagtgagagtgtgacactgtgtgtcagggcagtagtcacagtctgactgtgtcactcagacgcgtagtcacagagtgactcagtctctcagaatcgtagtcactgtgtgactctgagtctcagcgaggtagagactgtctgactcacagactcagtccagtagagagtgactgacactgagtctcagcctagtagacacactctgacacagagtctcagctaagtagtgacagtgtgactgtgacagtcaggggagcac

SEQ ID NO: 590 - DNA, 1036 bp, Artificial Sequence
  >BioblockX64_16
aataaatagtagacacacactgacacacacactcagtcaagtagacacacactgacacacacagtcagcttcgtagagtgagtctgactcagagtgtcagagtagtagtgacacactgacactgtcagtcagactggtagacacagactgactctcagtctcagcacagtagagagtgactgactgacagtctcagccaggtagagtgtgtctgacactgagactcagcaaagtagtgacagactgactgtgtcagtcaggaccgtagactgtctgtgacagtctcagtcagactcgtagtgtgagtctgacactcagactcagccacgtagagactgtctgacagagtctctcaggaacgtagacacacactgacacacacagtcaggcacgtagacacagactgacacacagtctcagcggcgtagacacacactgacacacacagtcagcgtagtagacactcagtgactcacactgtcaggtaagtagtgtctgtgtgacacacacactcagcaacgtagacacagtgtgacacactctgtcaggctagtagacacacagtgacacacacactcagccgagtagacacacagtgacactcacagtcagacgagtagactcacagtgacacacacagtcagagaagtagacacacactgacacactgactcagtaaagtagtcactcagtgacactgagtctcagagcggtagacacacactgacacactcactcagacctgtagacactctctgacacacacagtcagaacagtagacactctctgacagactgactcagggcagtagacacactctgacacagtgtgtcagacgcgtagacacacactgacacacacactcagaatcgtagacacagagtgacacacacagtcagcgaggtagacagtcagtgacacactctctcagtccagtagacacacactgacacacacactcagcctagtagacacacactgacacacacactcagctaagtagacacacactgacacacacactcaggggaaagg

SEQ ID NO: 591 - DNA, 940 bp, Artificial Sequence
  >BioblockX64_17
aaggaatagtagacacacactgacacacacactcagtcaagtagacacacactgacacacacactcagcttcgtagacagacagtgacacagtcagtcagagtagtagacacacactgacagactgactcagactggtagacacacactgacagtctcagtcagcacagtagacacacactgacagtcactctcagccaggtagacacacactgacagtcagagtcagcaaagtagacacacactgacagtgactctcaggaccgtagacacacactgacagtgagactcagactcgtagacacacactgactgtctcagtcagccacgtagacacacactgacactctgtctcaggaacgtagacacacactgacagtgagactcaggcacgtagacacacactgacagtgtcactcagcggcgtagacacacactgacagtgagactcagcgtagtagacacacactgacacacacactcaggtaagtagacacacactgacacagtcagtcagcaacgtagacacactctgacacacacactcaggctagtagacacacactgacacagagactcagccgagtagacactctctgacacacacagtcagacgagtagacacacactgacacacacactcagagaagtagtgtgagtgtgactgtcagtctcagtaaagtagtctgactgtgacagtcactgtcagagcggtagactgtgtgtgactgagtcactcagacctgtagacacacagtgacacagagagtcagaacagtagacacagtctgacacacacagtcagggcagtagacacacactgacactcacactcagacgcgtagtcacacactgactctgagactcagaatcgtagtcacacagtgacacacacactcagcgaggtagacacacactgaccagtctgttcagtccaaaac

SEQ ID NO: 592 - DNA, 16516 bp, Artificial Sequence
  >BioblockX1024_0
aataaatagtagacacacactgacacacacactcagtcaagtagacacacactgacacacacactcagcttcgtagactgagtgtgacagtgtctctcagagtagtagtctgtgactgactctctgtgtcagactggtagactcagtgtgacacagtgactcagcacagtagacacacactgacacacagactcagccaggtagtgagagactgactgagtgtgtcagcaaagtagtgtgtcagtgactctgtgactcaggaccgtagtgtctgtgtgacacacactgtcagactcgtagacacacactgacacacacactcagccacgtagacacacactgacacacacactcaggaacgtagacacacactgacacacacactcaggcacgtagagtcactctgacacacacactcagcggcgtagacacacactgacacacacactcagcgtagtagacacacactgacacacacactcaggtaagtagacacacactgacacacacactcagcaacgtagtgagtcactgacagactgtctcaggctagtagtcagagactgactcagtgtgtcagccgagtagtgtcacactgacacactcagtcagacgagtagactgagagtgacacacactgtcagagaagtagtgtcagtgtgacagagtgagtcagtaaagtagacacacactgacactcagtctcagagcggtagacagtctctgacagactcactcagacctgtagagacagtctgacagtgacagtcagaacagtagtgtctctctgacactcacagtcagggcagtagactgtgtctgacagactcagtcagacgcgtagtctctgtgtgactgtctgtgtcagaatcgtagtgtcacagtgactctgtgtctcagcgaggtagagtcacactgactgagacagtcagtccagtagacagactctgacagtgactgtcagcctagtagagtgtgactgactcacagtctcagctaagtagacacagactgacagacacactcaggggaaaggaatagtagacacactctgactgtctgtgtcagtcaagtagtgagagtgtgactctcactgtcagcttcgtagtcagtcactgactcactctctcagagtagtagacacactgtgactgagtgtctcagactggtagtgtgagtctgacacactgagtcagcacagtagagagtgactgactctcagagtcagccaggtagtcagtcagtgactgagacactcagcaaagtagacagtgagtgacagtgactgtcaggaccgtagactgtgtgtgacacagacactcagactcgtagtgtgtgactgacactcacactcagccacgtagtctgactgtgacagtcagtctcaggaacgtagacacagactgacactctctctcaggcacgtagagacacagtgactctcacagtcagcggcgtagtgtctgactgacacacagactcagcgtagtagacacagtgtgactctctctgtcaggtaagtagtcagtctgtgactcacagtctcagcaacgtagagtcagtctgactgtctgactcaggctagtagtctcactgtgactctctctgtcagccgagtagactgagagtgactctcacagtcagacgagtagtgagtctctgacacagtctctcagagaagtagacacagactgacacactgtgtcagtaaagtagactctctgtgacagactctgtcagagcggtagtcagtcactgacactgagagtcagacctgtagtgacagactgactgagacagtcagaacagtagtgtcagtctgacagtcactctcagggcagtagacagacagtgactgtcagtctcagacgcgtagtctctctgtgactgtctcagtcagaatcgtagagtgtgagtgacagagtctctcagcgaggtagactctgtgtgactctgtctctcagtccagtagactgagagtgactcagtgtctcagcctagtagacactgagtgactctcagtctcagctaagtagactctgagtgacagtgagtgtcaggggaaaacaatagtagacagacagtgacacacactgtcagtcaagtagacagagactgactgtcactgtcagcttcgtagtgactctgtgacagagagtctcagagtagtagtcactctgtgacacagtgtgtcagactggtagtgtcagtgtgacactgtcagtcagcacagtagtgtgtcagtgactctgactctcagccaggtagactctctctgacagtgtgagtcagcaaagtagactcagagtgacagacacactcaggaccgtagtgacagtgtgacactgtcactcagactcgtagactctgtgtgacagtgagagtcagccacgtagtcagtgagtgactcagtgtgtcaggaacgtagagtcacagtgacacagtgagtcaggcacgtagagtgtcactgactctgtcagtcagcggcgtagtcagtctgtgactctctgtctcagcgtagtagactgagtgtgacagacacactcaggtaagtagtgtgagagtgactcacagtctcagcaacgtagactgtgagtgactgacactgtcaggctagtagagagtcagtgactcagagtctcagccgagtagtgtcacactgactgactctctcagacgagtagagtctctctgactcagagtgtcagagaagtagactgagtgtgactctcactctcagtaaagtagtgtcagagtgacagtgacagtcagagcggtagtgtgactgtgacagtctcagtcagacctgtagtgtgtctctgactgtcagactcagaacagtagactctctgtgactcagacagtcagggcagtagtgtgagtgtgacactcagtgtcagacgcgtagtctgtgagtgactgagacagtcagaatcgtagtgagagtctgacagagactgtcagcgaggtagagagtgtgtgactctcagactcagtccagtagagagagagtgacagagagtgtcagcctagtagacacagtctgacagagtctctcagctaagtagtcagagtctgactgactgagtcaggggataaaaatagtagagtctgtgtgactgtgagtctcagtcaagtagacacacagtgacactctcactcagcttcgtagagacacagtgactgtgtgtctcagagtagtagtgactctctgactctgtcagtcagactggtagactgtctctgactgtctgtctcagcacagtagtgtctctctgacagtgtcagtcagccaggtagactgacagtgactctctgagtcagcaaagtagtgagactctgacagtgacactcaggaccgtagagtcactgtgactcagtctgtcagactcgtagtcactgtctgacagtgtgactcagccacgtagtgtgacagtgacagtgtgagtcaggaacgtagtcagtgactgactgagagagtcaggcacgtagagacagagtgacacagacactcagcggcgtagtgagagtgtgacactgagactcagcgtagtagacagtcactgactgagtctgtcaggtaagtagactgtcagtgactgactctgtcagcaacgtagtctctctgtgactgagacactcaggctagtagtcagagactgacagtgtgactcagccgagtagagacacagtgacagtgagtctcagacgagtagtgacagagtgactctgtcactcagagaagtagtctctcactgactctgactctcagtaaagtagtgagagtgtgactgtgtcactcagagcggtagtcagacagtgactgtgtgtgtcagacctgtagagagactctgacagtgagagtcagaacagtagagtctgtctgacactctcagtcagggcagtagtctcagactgactgtctgagtcagacgcgtagactcagtgtgacagacactctcagaatcgtagtcacagtctgactgtgtcactcagcgaggtagacactgagtgacactctgagtcagtccagtagtcagagtctgactgtgacactcagcctagtagtgagacagtgacagtgtcactcagctaagtagacagagtgtgactgagtctgtcaggggaacgaaatagtagagtgacagtgactcagagactcagtcaagtagactctgagtgacactgtgtgtcagcttcgtagtctgacactgactgtgacagtcagagtagtagtcagacagtgacagactgactcagactggtagagtcagactgacactcagactcagcacagtagagacagagtgactgtctcactcagccaggtagtgacagtgtgacagtgacagtcagcaaagtagtgagagactgactctcactgtcaggaccgtagtgtgtcagtgactcacactgtcagactcgtagtgtctctgtgacacactgtgtcagccacgtagagtctcagtgactctcagactcaggaacgtagagacactctgacagtgtgtctcaggcacgtagagagagactgactctctcagtcagcggcgtagagtcacagtgactgtgagagtcagcgtagtagtgtcagtgtgactctgactctcaggtaagtagtgtgtgtctgacagtcacagtcagcaacgtagtcacactctgacagagacactcaggctagtagacactgactgacactcactctcagccgagtagagtgagagtgacagacagtgtcagacgagtagtgagactctgactgagtgagtcagagaagtagtctgagtgtgacagtgtgagtcagtaaagtagtctgagagtgacacagtgtctcagagcggtagagagactgtgactctcagtgtcagacctgtagactctgagtgactctgtgtctcagaacagtagtgagactctgactcagacactcagggcagtagagtgagtctgacacactcagtcagacgcgtagtgtgagtctgacagtctcactcagaatcgtagagactgagtgactgagactctcagcgaggtagtgacagtgtgactctgtctctcagtccagtagagtgagtctgacagagagactcagcctagtagactctctgtgacacagagactcagctaagtagacacagactgacagagacagtcaggggaactgaatagtagagtctgtctgactgtctgagtcagtcaagtagagagtgagtgacagtcacagtcagcttcgtagtgagagagtgactgtcactgtcagagtagtagtctgtgagtgacactcacactcagactggtagtgagagactgactgtgtcagtcagcacagtagtgactgagtgacactgacagtcagccaggtagagagtgagtgactgagagactcagcaaagtagacagagagtgacacacagactcaggaccgtagtgagtcactgactgtgagactcagactcgtagtctgagtctgactgactctgtcagccacgtagactcactctgacacactgagtcaggaacgtagtcacacagtgactctctcactcaggcacgtagagactgactgacagagacagtcagcggcgtagtgagagtctgactctcagtctcagcgtagtagacagagagtgacagactctgtcaggtaagtagtgactgagtgacactcactctcagcaacgtagtcactgactgactgtctcactcaggctagtagtgtgacactgactcacacactcagccgagtagtctgtgtctgactcactgagtcagacgagtagtctctgagtgactctgagactcagagaagtagacacagtgtgactcactgactcagtaaagtagagacagactgacactgtgactcagagcggtagacacactctgactctctgactcagacctgtagacacagtctgacacacacactcagaacagtagacagagactgactcacacagtcagggcagtagacagagagtgacacagtcactcagacgcgtagagactgtctgactcagactgtcagaatcgtagtcacacactgacactgacagtcagcgaggtagtgagtgtctgacacactcagtcagtccagtagactcactctgactgagagagtcagcctagtagtcacacagtgactctgtcagtcagctaagtagacagagtgtgactgtcagtctcaggggaagcgaatagtagtctctcagtgactctgagagtcagtcaagtagtcagagtctgacactgtgtgtcagcttcgtagagtctcactgacacagtgagtcagagtagtagtgagagagtgacagagagagtcagactggtagtgacagactgacagactgagtcagcacagtagtcagacactgacagtgagagtcagccaggtagtgtcagtgtgacagacacactcagcaaagtagagtctctctgacactgtgagtcaggaccgtagtctcactctgactcagactctcagactcgtagtgactgagtgactgactcagtcagccacgtagtcacagtgtgactgagagagtcaggaacgtagtgtcagtctgactgtgtctgtcaggcacgtagtcagagtctgacagagtgagtcagcggcgtagtcagagtctgacactctgagtcagcgtagtagtctgagactgacacactcactcaggtaagtagtgtgagactgactgtcacagtcagcaacgtagtctgactgtgacacagagtctcaggctagtagacagactgtgactgactgtctcagccgagtagtctcagtctgactgagtcactcagacgagtagtgagacactgacactgtgactcagagaagtagtctgtctgtgacactcacactcagtaaagtagtgtcagtgtgacagagtgagtcagagcggtagtcacacactgactcactctgtcagacctgtagtgactctctgactgactgtctcagaacagtagacagtgtgtgacacacacagtcagggcagtagtctgagagtgactgtgtcagtcagacgcgtagtctgagactgacactgtcactcagaatcgtagactgtgagtgacagtgagactcagcgaggtagtgtcacactgacactcactctcagtccagtagtgtgtgagtgactctcagtctcagcctagtagacacacactgacacagtgtctcagctaagtagtgagagtctgactcactctgtcaggggagctaaatagtagactgtgtctgactgtgtgagtcagtcaagtagactgacagtgactcacagactcagcttcgtagtgagtgagtgactgtgagtgtcagagtagtagtctcagactgactgtgtgagtcagactggtagactcacactgacactgactctcagcacagtagacacacagtgactcagtcactcagccaggtagagtgtcactgactcactctctcagcaaagtagtcactgactgacactcagtctcaggaccgtagtctgtgagtgacactcacactcagactcgtagagagactgtgacacagtgagtcagccacgtagtctcacagtgacactgtgtctcaggaacgtagagtctgagtgacagtctgactcaggcacgtagtcagtctgtgactctctctgtcagcggcgtagagtgtctctgactgagtgagtcagcgtagtagtcagacagtgacactgagagtcaggtaagtagtgtgagactgacagtcagagtcagcaacgtagtgactcagtgacagagtctctcaggctagtagtctgtcactgactgtctcagtcagccgagtagtgtgtgagtgacagtctctctcagacgagtagagactgtctgactgagactgtcagagaagtagtgactcagtgactgacactctcagtaaagtagacagactgtgacactgtcactcagagcggtagtcactcagtgacacactctgtcagacctgtagtgtctgtgtgactgtctctctcagaacagtagacactgtctgactgacagtgtcagggcagtagtctctctgtgacacagtctgtcagacgcgtagtcacagagtgactgactctctcagaatcgtagactgactgtgacacagtctgtcagcgaggtagtgagacactgacagtgagtgtcagtccagtagagtctgagtgactctcagactcagcctagtagtctgtgtgtgactgtgagtgtcagctaagtagtcactgtctgacagactgtctcaggggaggcaaatagtagagtctctctgacagtcagactcagtcaagtagtcacacagtgactcactctctcagcttcgtagagtctgtgtgacagagtgagtcagagtagtagtgactctgtgacacactgtctcagactggtagagtgtctctgactgactgagtcagcacagtagtgtctctgtgacactcacagtcagccaggtagtgagtctgtgactgactgtgtcagcaaagtagtctgagagtgacagtcacactcaggaccgtagtcagtctgtgacacacactctcagactcgtagtctctgagtgactgagtgtctcagccacgtagactcacactgacagtctgactcaggaacgtagactgacactgactcagtgagtcaggcacgtagtgactcactgactcacacactcagcggcgtagtctgtgtgtgactctgagtgtcagcgtagtagtgacactgtgactgtctctgtcaggtaagtagtcagacactgacactgtcactcagcaacgtagactcagtgtgacactgacagtcaggctagtagtgacagagtgactctctgagtcagccgagtagactcagagtgacagagacagtcagacgagtagacagacagtgactgagtgactcagagaagtagtgagtcactgacagtcagtgtcagtaaagtagtgactgactgactgtcacagtcagagcggtagacagagactgactgagtgactcagacctgtagagagacactgactgtcacagtcagaacagtagtcagacagtgacagagtcagtcagggcagtagtctcacagtgactctctctgtcagacgcgtagacagtcagtgactgtgactgtcagaatcgtagtgtctcagtgactcactcagtcagcgaggtagactgacagtgacactgtctctcagtccagtagtcagagagtgacagtcacagtcagcctagtagtgtcactctgactgtgtgtgtcagctaagtagtgagtcactgactgagagtctcaggggaacctaatagtagtgagtctgtgacacactcagtcagtcaagtagactgtgtgtgacactgtgtgtcagcttcgtagtcactctctgacacacagtgtcagagtagtagagagacactgacagacactctcagactggtagtctgtgtgtgactgtctctgtcagcacagtagtctcagagtgacagtcactgtcagccaggtagtcagacagtgactgacagtctcagcaaagtagagtctgtgtgacactcactgtcaggaccgtagtgtgacagtgacacagtctctcagactcgtagagacactgtgacacagtctgtcagccacgtagagactcactgacactgtgagtcaggaacgtagacactgtctgactcagtcagtcaggcacgtagagagagtctgacagactgagtcagcggcgtagtctgtctgtgacactgtgtctcagcgtagtagacactgtctgacagtctgactcaggtaagtagagagacagtgacagtgagtgtcagcaacgtagagagactctgacagtgagagtcaggctagtagagtgagagtgacacacacagtcagccgagtagtgacagtctgacactctctgtcagacgagtagtgacacactgactgtgtctctcagagaagtagtgtgtgagtgacacactgtgtcagtaaagtagacagagtgtgacacactcactcagagcggtagtgtcagtgtgacagtctctgtcagacctgtagagactcagtgactctgtgactcagaacagtagtctgagtctgacagtctgactcagggcagtagacagactctgacagactcagtcagacgcgtagactgtgtctgactcagacagtcagaatcgtagactcacactgactctgactgtcagcgaggtagagtcagagtgacacagtcagtcagtccagtagtgacagagtgactgtctctctcagcctagtagagtgtgactgacacagtgactcagctaagtagagtcactgtgacagtgtgactcaggggacgtaaatagtagactcagtgtgacactgagagtcagtcaagtagagtgagtgtgactgtgagagtcagcttcgtagagacagagtgactgtgacactcagagtagtagagacagagtgactcagtgtgtcagactggtagactgacactgacacacagagtcagcacagtagagactgtgtgacacacacagtcagccaggtagtgactgagtgacacacacactcagcaaagtagagtgacagtgacacagacagtcaggaccgtagtctcagtgtgactctctctctcagactcgtagagagactctgactgtgtgagtcagccacgtagtgtgactgtgactgagtgtgtcaggaacgtagagagtcagtgacactctgtctcaggcacgtagtctgtctgtgacacagagactcagcggcgtagtgtgtgactgacagtcactctcagcgtagtagagactcagtgacacagtcagtcaggtaagtagagactgtgtgacactgagtctcagcaacgtagagagtctgtgactgtgtctgtcaggctagtagtcagtgtgtgactcacagtgtcagccgagtagacagagactgactcagtgtctcagacgagtagagtctctctgacacagagagtcagagaagtagacagtgactgacagagacagtcagtaaagtagagtgacactgacagtctgactcagagcggtagtgacagagtgactgacagagtcagacctgtagtgtgactctgactgactgactcagaacagtagtcagagtctgactcactgagtcagggcagtagactgagagtgacactctctgtcagacgcgtagtctcacagtgactgtctctgtcagaatcgtagtgtctgactgacactgtctgtcagcgaggtagtgacagactgactctgagagtcagtccagtagagagagactgacagagtcagtcagcctagtagtgtctgagtgactgagtctgtcagctaagtagactctgagtgacacagtgtctcaggggaaacaaatagtagtctcagagtgacagagagtgtcagtcaagtagtctgtgactgacagtgagtgtcagcttcgtagacagtcactgacagtcagtgtcagagtagtagacacactgtgacacactctgtcagactggtagactgtctgtgactcagtgagtcagcacagtagtgtgagtgtgactgactgtgtcagccaggtagagtgtcagtgactctctgagtcagcaaagtagactcagactgactgacagtgtcaggaccgtagagtcacagtgacactctcactcagactcgtagtcagtctgtgacacactctgtcagccacgtagtctgagagtgactcactgtctcaggaacgtagtgtgtcactgactgagacagtcaggcacgtagtgagtctgtgactgtctgtgtcagcggcgtagtcactcagtgactgtgtgagtcagcgtagtagacagtgagtgacactgactctcaggtaagtagtcactcactgactgacagactcagcaacgtagtctctcagtgactgagtcagtcaggctagtagacagtgactgactcagtgagtcagccgagtagtgagtgagtgactctctgtctcagacgagtagtgactctctgacagagacagtcagagaagtagtgtgtcactgactgtcactgtcagtaaagtagtcagacagtgacagtgactgtcagagcggtagtctcacactgactgtgtgactcagacctgtagagtgacagtgacagagacagtcagaacagtagtgtcagactgacagtctgtctcagggcagtagtgagagactgacagactctctcagacgcgtagagtgacagtgactgactgagtcagaatcgtagtcagtgagtgacacagtgtctcagcgaggtagtgacagtctgacacactcactcagtccagtagactgactgtgactgtgtcactcagcctagtagagtgagactgactgtgagtctcagctaagtagagacacactgacagacacactcaggggactacaatagtagtgtctgactgactgtgtgtctcagtcaagtagtcagagtgtgactctgagtctcagcttcgtagagacagtctgacagactcactcagagtagtagtgtgtctctgacacacactctcagactggtagtctgacagtgactgacactctcagcacagtagtgacacactgacagacagtgtcagccaggtagtctgagactgacagtgacagtcagcaaagtagagactcactgactcagactctcaggaccgtagacagagtgtgactcagtctgtcagactcgtagagtcagtctgacacagtctctcagccacgtagtgagactgtgactcacactgtcaggaacgtagtcagacactgactgtgtcactcaggcacgtagacagactctgactgtcactgtcagcggcgtagtgtgtctctgactgagtgtctcagcgtagtagtcagtctgtgacagtcagtgtcaggtaagtagtgactcagtgactgtgtctgtcagcaacgtagactctcactgacagtcagtgtcaggctagtagtcagagagtgactgacacagtcagccgagtagacacagagtgacagtgagtctcagacgagtagtcactcactgactcactcagtcagagaagtagtcactcactgactcagacagtcagtaaagtagacactctgtgacactgacactcagagcggtagtgacagtgtgacactcactgtcagacctgtagtgtcacagtgacacacacagtcagaacagtagacactctctgactgtctctgtcagggcagtagagtgtgactgactcactcactcagacgcgtagtgagagtgtgacagagagagtcagaatcgtagagtgacactgacactgactctcagcgaggtagagagtgtgtgactgacacactcagtccagtagtgtgtgtgtgactcagtgagtcagcctagtagtcacagactgacagactcactcagctaagtagacactgagtgacactgagactcaggggagagaaatagtagtcacagtctgacacactgtgtcagtcaagtagagtgacactgacagtgagagtcagcttcgtagtcactgtctgacacagtctgtcagagtagtagtgtgtctgtgactctgacagtcagactggtagtcacagagtgacacactgactcagcacagtagtcagactctgacacacactctcagccaggtagacacagagtgacagactgtgtcagcaaagtagagtctcagtgacagtgtctctcaggaccgtagacactgactgacagacactgtcagactcgtagagagagtctgacacagtcactcagccacgtagtgtgtcagtgacacacactgtcaggaacgtagtcactgtctgactgtcactgtcaggcacgtagtctctcagtgactctgagtgtcagcggcgtagagtgagtctgacagtgacactcagcgtagtagtcactctctgacacactgtgtcaggtaagtagacacagagtgactgtcacagtcagcaacgtagagacagtgtgacactgagtgtcaggctagtagtcagtgagtgacacacactgtcagccgagtagacagtcagtgacagagagactcagacgagtagagacagagtgacagtgtctgtcagagaagtagactgtgactgactgtctgactcagtaaagtagtgtgacagtgacacagtcactcagagcggtagagagtcactgacactgacagtcagacctgtagtgagacactgactcagactgtcagaacagtagtgacacactgacagtcactgtcagggcagtagagtctctctgactcactgtctcagacgcgtagacagacagtgacagagtgagtcagaatcgtagacactgtctgacagacacagtcagcgaggtagactgagagtgactctcacactcagtccagtagacagtcactgacacactgagtcagcctagtagactcactctgactcagtgagtcagctaagtagtctgactgtgacactcagagtcaggggaccagaatagtagacacagtgtgactgagagactcagtcaagtagagactgagtgactcagtctctcagcttcgtagagactctgtgactctgtgtctcagagtagtagactgtcactgacactgacagtcagactggtagacacagagtgacacacagagtcagcacagtagtcactctctgactcagagtgtcagccaggtagagtgtcagtgactgtctgagtcagcaaagtagagtgagagtgactctcagactcaggaccgtagtcagacagtgacagtgagtgtcagactcgtagtctgactgtgacactgactctcagccacgtagagtctcactgacagagagtctcaggaacgtagagtgagtctgacacacagactcaggcacgtagagtgacagtgactctctgagtcagcggcgtagactctgagtgacagactctctcagcgtagtagacagactgtgactcagtcagtcaggtaagtagtctcactctgactctctgtctcagcaacgtagacacagtctgactgactctgtcaggctagtagtcactgtctgacacacacactcagccgagtagtgtctgtctgacacagtcactcagacgagtagacagagagtgactctgtgtgtcagagaagtagagagtctgtgacagtgtgagtcagtaaagtagagtcacagtgactcactgactcagagcggtagactgtcagtgactgtcagagtcagacctgtagacagagagtgactctgagactcagaacagtagtcacacagtgactcagacagtcagggcagtagtgagtgtctgacacactgactcagacgcgtagtcagtcactgacactgactgtcagaatcgtagactctgagtgactgtctgagtcagcgaggtagactctgactgacacagactgtcagtccagtagtgtgtgagtgacagagtctgtcagcctagtagagactgtctgactgacagtgtcagctaagtagtgactcactgacactcagtctcaggggaagaaaatagtagagagacactgacagactgtgtcagtcaagtagtgagtgtgtgacacagtgagtcagcttcgtagactgtcactgacagtctgagtcagagtagtagtgtctgagtgacactgagagtcagactggtagacactgagtgacagtcactctcagcacagtagtcagtgtgtgactcactcactcagccaggtagtgagactctgacagtcagtctcagcaaagtagtctgtgagtgactctgagtgtcaggaccgtagacactgtctgacagagagtctcagactcgtagtctctctgtgactgtgtcactcagccacgtagtcagacactgactcactgagtcaggaacgtagagtgagagtgacacagtgactcaggcacgtagacagagactgacagactgagtcagcggcgtagagtgactgtgactcactcagtcagcgtagtagtgacactctgacacagtcagtcaggtaagtagacagagagtgacagagtctgtcagcaacgtagtgtctcagtgactgtctcagtcaggctagtagtctctctctgacacagtgagtcagccgagtagagactctctgactgtcacactcagacgagtagacactcactgactgtcacagtcagagaagtagtgacacagtgacagtgagactcagtaaagtagtcacagtctgactctctgagtcagagcggtagtgtgtcactgacagtcactctcagacctgtagtctgagtgtgacagagactctcagaacagtagtgagagtgtgacactgtgtgtcagggcagtagtcacagtctgactgtgtcactcagacgcgtagtcacagagtgactcagtctctcagaatcgtagtcactgtgtgactctgagtctcagcgaggtagagactgtctgactcacagactcagtccagtagagagtgactgacactgagtctcagcctagtagacacactctgacacagagtctcagctaagtagtgacagtgtgactgtgacagtcaggggagcac

SEQ ID NO: 593 - DNA, 1972 bp, Artificial Sequence
  >BioblockX1024_1
aataaatagtagacacacactgacacacacactcagtcaagtagacacacactgacacacacagtcagcttcgtagagtgagtctgactcagagtgtcagagtagtagtgacacactgacactgtcagtcagactggtagacacagactgactctcagtctcagcacagtagagagtgactgactgacagtctcagccaggtagagtgtgtctgacactgagactcagcaaagtagtgacagactgactgtgtcagtcaggaccgtagactgtctgtgacagtctcagtcagactcgtagtgtgagtctgacactcagactcagccacgtagagactgtctgacagagtctctcaggaacgtagacacacactgacacacacagtcaggcacgtagacacagactgacacacagtctcagcggcgtagacacacactgacacacacagtcagcgtagtagacactcagtgactcacactgtcaggtaagtagtgtctgtgtgacacacacactcagcaacgtagacacagtgtgacacactctgtcaggctagtagacacacagtgacacacacactcagccgagtagacacacagtgacactcacagtcagacgagtagactcacagtgacacacacagtcagagaagtagacacacactgacacactgactcagtaaagtagtcactcagtgacactgagtctcagagcggtagacacacactgacacactcactcagacctgtagacactctctgacacacacagtcagaacagtagacactctctgacagactgactcagggcagtagacacactctgacacagtgtgtcagacgcgtagacacacactgacacacacactcagaatcgtagacacagagtgacacacacagtcagcgaggtagacagtcagtgacacactctctcagtccagtagacacacactgacacacacactcagcctagtagacacacactgacacacacactcagctaagtagacacacactgacacacacactcaggggaaaggaatagtagacacacactgacacacacactcagtcaagtagacacacactgacacacacactcagcttcgtagacagacagtgacacagtcagtcagagtagtagacacacactgacagactgactcagactggtagacacacactgacagtctcagtcagcacagtagacacacactgacagtcactctcagccaggtagacacacactgacagtcagagtcagcaaagtagacacacactgacagtgactctcaggaccgtagacacacactgacagtgagactcagactcgtagacacacactgactgtctcagtcagccacgtagacacacactgacactctgtctcaggaacgtagacacacactgacagtgagactcaggcacgtagacacacactgacagtgtcactcagcggcgtagacacacactgacagtgagactcagcgtagtagacacacactgacacacacactcaggtaagtagacacacactgacacagtcagtcagcaacgtagacacactctgacacacacactcaggctagtagacacacactgacacagagactcagccgagtagacactctctgacacacacagtcagacgagtagacacacactgacacacacactcagagaagtagtgtgagtgtgactgtcagtctcagtaaagtagtctgactgtgacagtcactgtcagagcggtagactgtgtgtgactgagtcactcagacctgtagacacacagtgacacagagagtcagaacagtagacacagtctgacacacacagtcagggcagtagacacacactgacactcacactcagacgcgtagtcacacactgactctgagactcagaatcgtagtcacacagtgacacacacactcagcgaggtagacacacactgaccagtctgttcaggggaaaac

SEQ ID NO: 594 - DNA, 16 bp, Artificial Sequence
  00000000 Library A
gtagacacacactgac

SEQ ID NO: 595 - DNA, 16 bp, Artificial Sequence
  00000001 Library A
gtagacacacagtgac

SEQ ID NO: 596 - DNA, 16 bp, Artificial Sequence
  00000010 Library A
gtagacacactctgac

SEQ ID NO: 597 - DNA, 16 bp, Artificial Sequence
  00000011 Library A
gtagacacactgtgac

SEQ ID NO: 598 - DNA, 16 bp, Artificial Sequence
  00000100 Library A
gtagacacagactgac

SEQ ID NO: 599 - DNA, 16 bp, Artificial Sequence
  00000101 Library A
gtagacacagagtgac

SEQ ID NO: 600 - DNA, 16 bp, Artificial Sequence
  00000110 Library A
gtagacacagtctgac

SEQ ID NO: 601 - DNA, 16 bp, Artificial Sequence
  00000111 Library A
gtagacacagtgtgac

SEQ ID NO: 602 - DNA, 16 bp, Artificial Sequence
  00001000 Library A
gtagacactcactgac

SEQ ID NO: 603 - DNA, 16 bp, Artificial Sequence
  00001001 Library A
gtagacactcagtgac

SEQ ID NO: 604 - DNA, 16 bp, Artificial Sequence
  00001010 Library A
gtagacactctctgac

SEQ ID NO: 605 - DNA, 16 bp, Artificial Sequence
  00001011 Library A
gtagacactctgtgac

SEQ ID NO: 606 - DNA, 16 bp, Artificial Sequence
  00001100 Library A
gtagacactgactgac

SEQ ID NO: 607 - DNA, 16 bp, Artificial Sequence
  00001101 Library A
gtagacactgagtgac

SEQ ID NO: 608 - DNA, 16 bp, Artificial Sequence
  00001110 Library A
gtagacactgtctgac

SEQ ID NO: 609 - DNA, 16 bp, Artificial Sequence
  00001111 Library A
gtagacactgtgtgac

SEQ ID NO: 610 - DNA, 16 bp, Artificial Sequence
  00010000 Library A
gtagacagacactgac

SEQ ID NO: 611 - DNA, 16 bp, Artificial Sequence
  00010001 Library A
gtagacagacagtgac

SEQ ID NO: 612 - DNA, 16 bp, Artificial Sequence
  00010010 Library A
gtagacagactctgac

SEQ ID NO: 613 - DNA, 16 bp, Artificial Sequence
  00010011 Library A
gtagacagactgtgac

SEQ ID NO: 614 - DNA, 16 bp, Artificial Sequence
  00010100 Library A
gtagacagagactgac

SEQ ID NO: 615 - DNA, 16 bp, Artificial Sequence
  00010101 Library A
gtagacagagagtgac

SEQ ID NO: 616 - DNA, 16 bp, Artificial Sequence
  00010110 Library A
gtagacagagtctgac

SEQ ID NO: 617 - DNA, 16 bp, Artificial Sequence
  00010111 Library A
gtagacagagtgtgac

SEQ ID NO: 618 - DNA, 16 bp, Artificial Sequence
  00011000 Library A
gtagacagtcactgac

SEQ ID NO: 619 - DNA, 16 bp, Artificial Sequence
  00011001 Library A
gtagacagtcagtgac

SEQ ID NO: 620 - DNA, 16 bp, Artificial Sequence
  00011010 Library A
gtagacagtctctgac

SEQ ID NO: 621 - DNA, 16 bp, Artificial Sequence
  00011011 Library A
gtagacagtctgtgac

SEQ ID NO: 622 - DNA, 16 bp, Artificial Sequence
  00011100 Library A
gtagacagtgactgac

SEQ ID NO: 623 - DNA, 16 bp, Artificial Sequence
  00011101 Library A
gtagacagtgagtgac

SEQ ID NO: 624 - DNA, 16 bp, Artificial Sequence
  00011110 Library A
gtagacagtgtctgac

SEQ ID NO: 625 - DNA, 16 bp, Artificial Sequence
  00011111 Library A
gtagacagtgtgtgac

SEQ ID NO: 626 - DNA, 16 bp, Artificial Sequence
  00100000 Library A
gtagactcacactgac

SEQ ID NO: 627 - DNA, 16 bp, Artificial Sequence
  00100001 Library A
gtagactcacagtgac

SEQ ID NO: 628 - DNA, 16 bp, Artificial Sequence
  00100010 Library A
gtagactcactctgac

SEQ ID NO: 629 - DNA, 16 bp, Artificial Sequence
  00100011 Library A
gtagactcactgtgac

SEQ ID NO: 630 - DNA, 16 bp, Artificial Sequence
  00100100 Library A
gtagactcagactgac

SEQ ID NO: 631 - DNA, 16 bp, Artificial Sequence
  00100101 Library A
gtagactcagagtgac

SEQ ID NO: 632 - DNA, 16 bp, Artificial Sequence
  00100110 Library A
gtagactcagtctgac

SEQ ID NO: 633 - DNA, 16 bp, Artificial Sequence
  00100111 Library A
gtagactcagtgtgac

SEQ ID NO: 634 - DNA, 16 bp, Artificial Sequence
  00101000 Library A
gtagactctcactgac

SEQ ID NO: 635 - DNA, 16 bp, Artificial Sequence
  00101001 Library A
gtagactctcagtgac

SEQ ID NO: 636 - DNA, 16 bp, Artificial Sequence
  00101010 Library A
gtagactctctctgac

SEQ ID NO: 637 - DNA, 16 bp, Artificial Sequence
  00101011 Library A
gtagactctctgtgac

SEQ ID NO: 638 - DNA, 16 bp, Artificial Sequence
  00101100 Library A
gtagactctgactgac

SEQ ID NO: 639 - DNA, 16 bp, Artificial Sequence
  00101101 Library A
gtagactctgagtgac

SEQ ID NO: 640 - DNA, 16 bp, Artificial Sequence
  00101110 Library A
gtagactctgtctgac

SEQ ID NO: 641 - DNA, 16 bp, Artificial Sequence
  00101111 Library A
gtagactctgtgtgac

SEQ ID NO: 642 - DNA, 16 bp, Artificial Sequence
  00110000 Library A
gtagactgacactgac

SEQ ID NO: 643 - DNA, 16 bp, Artificial Sequence
  00110001 Library A
gtagactgacagtgac

SEQ ID NO: 644 - DNA, 16 bp, Artificial Sequence
  00110010 Library A
gtagactgactctgac

SEQ ID NO: 645 - DNA, 16 bp, Artificial Sequence
  00110011 Library A
gtagactgactgtgac

SEQ ID NO: 646 - DNA, 16 bp, Artificial Sequence
  00110100 Library A
gtagactgagactgac

SEQ ID NO: 647 - DNA, 16 bp, Artificial Sequence
  00110101 Library A
gtagactgagagtgac

SEQ ID NO: 648 - DNA, 16 bp, Artificial Sequence
  00110110 Library A
gtagactgagtctgac

SEQ ID NO: 649 - DNA, 16 bp, Artificial Sequence
  00110111 Library A
gtagactgagtgtgac

SEQ ID NO: 650 - DNA, 16 bp, Artificial Sequence
  00111000 Library A
gtagactgtcactgac

SEQ ID NO: 651 - DNA, 16 bp, Artificial Sequence
  00111001 Library A
gtagactgtcagtgac

SEQ ID NO: 652 - DNA, 16 bp, Artificial Sequence
  00111010 Library A
gtagactgtctctgac

SEQ ID NO: 653 - DNA, 16 bp, Artificial Sequence
  00111011 Library A
gtagactgtctgtgac

SEQ ID NO: 654 - DNA, 16 bp, Artificial Sequence
  00111100 Library A
gtagactgtgactgac

SEQ ID NO: 655 - DNA, 16 bp, Artificial Sequence
  00111101 Library A
gtagactgtgagtgac

SEQ ID NO: 656 - DNA, 16 bp, Artificial Sequence
  00111110 Library A
gtagactgtgtctgac

SEQ ID NO: 657 - DNA, 16 bp, Artificial Sequence
  00111111 Library A
gtagactgtgtgtgac

SEQ ID NO: 658 - DNA, 16 bp, Artificial Sequence
  01000000 Library A
gtagagacacactgac

SEQ ID NO: 659 - DNA, 16 bp, Artificial Sequence
  01000001 Library A
gtagagacacagtgac

SEQ ID NO: 660 - DNA, 16 bp, Artificial Sequence
  01000010 Library A
gtagagacactctgac

SEQ ID NO: 661 - DNA, 16 bp, Artificial Sequence
  01000011 Library A
gtagagacactgtgac

SEQ ID NO: 662 - DNA, 16 bp, Artificial Sequence
  01000100 Library A
gtagagacagactgac

SEQ ID NO: 663 - DNA, 16 bp, Artificial Sequence
  01000101 Library A
gtagagacagagtgac

SEQ ID NO: 664 - DNA, 16 bp, Artificial Sequence
  01000110 Library A
gtagagacagtctgac

SEQ ID NO: 665 - DNA, 16 bp, Artificial Sequence
  01000111 Library A
gtagagacagtgtgac

SEQ ID NO: 666 - DNA, 16 bp, Artificial Sequence
  01001000 Library A
gtagagactcactgac

SEQ ID NO: 667 - DNA, 16 bp, Artificial Sequence
  01001001 Library A
gtagagactcagtgac

SEQ ID NO: 668 - DNA, 16 bp, Artificial Sequence
  01001010 Library A
gtagagactctctgac

SEQ ID NO: 669 - DNA, 16 bp, Artificial Sequence
  01001011 Library A
gtagagactctgtgac

SEQ ID NO: 670 - DNA, 16 bp, Artificial Sequence
  01001100 Library A
gtagagactgactgac

SEQ ID NO: 671 - DNA, 16 bp, Artificial Sequence
  01001101 Library A
gtagagactgagtgac

SEQ ID NO: 672 - DNA, 16 bp, Artificial Sequence
  01001110 Library A
gtagagactgtctgac

SEQ ID NO: 673 - DNA, 16 bp, Artificial Sequence
  01001111 Library A
gtagagactgtgtgac

SEQ ID NO: 674 - DNA, 16 bp, Artificial Sequence
  01010000 Library A
gtagagagacactgac

SEQ ID NO: 675 - DNA, 16 bp, Artificial Sequence
  01010001 Library A
gtagagagacagtgac

SEQ ID NO: 676 - DNA, 16 bp, Artificial Sequence
  01010010 Library A
gtagagagactctgac

SEQ ID NO: 677 - DNA, 16 bp, Artificial Sequence
  01010011 Library A
gtagagagactgtgac

SEQ ID NO: 678 - DNA, 16 bp, Artificial Sequence
  01010100 Library A
gtagagagagactgac

SEQ ID NO: 679 - DNA, 16 bp, Artificial Sequence
  01010101 Library A
gtagagagagagtgac

SEQ ID NO: 680 - DNA, 16 bp, Artificial Sequence
  01010110 Library A
gtagagagagtctgac

SEQ ID NO: 681 - DNA, 16 bp, Artificial Sequence
  01010111 Library A
gtagagagagtgtgac

SEQ ID NO: 682 - DNA, 16 bp, Artificial Sequence
  01011000 Library A
gtagagagtcactgac

SEQ ID NO: 683 - DNA, 16 bp, Artificial Sequence
  01011001 Library A
gtagagagtcagtgac

SEQ ID NO: 684 - DNA, 16 bp, Artificial Sequence
  01011010 Library A
gtagagagtctctgac

SEQ ID NO: 685 - DNA, 16 bp, Artificial Sequence
  01011011 Library A
gtagagagtctgtgac

SEQ ID NO: 686 - DNA, 16 bp, Artificial Sequence
  01011100 Library A
gtagagagtgactgac

SEQ ID NO: 687 - DNA, 16 bp, Artificial Sequence
  01011101 Library A
gtagagagtgagtgac

SEQ ID NO: 688 - DNA, 16 bp, Artificial Sequence
  01011110 Library A
gtagagagtgtctgac

SEQ ID NO: 689 - DNA, 16 bp, Artificial Sequence
  01011111 Library A
gtagagagtgtgtgac

SEQ ID NO: 690 - DNA, 16 bp, Artificial Sequence
  01100000 Library A
gtagagtcacactgac

SEQ ID NO: 691 - DNA, 16 bp, Artificial Sequence
  01100001 Library A
gtagagtcacagtgac

SEQ ID NO: 692 - DNA, 16 bp, Artificial Sequence
  01100010 Library A
gtagagtcactctgac

SEQ ID NO: 693 - DNA, 16 bp, Artificial Sequence
  01100011 Library A
gtagagtcactgtgac

SEQ ID NO: 694 - DNA, 16 bp, Artificial Sequence
  01100100 Library A
gtagagtcagactgac

SEQ ID NO: 695 - DNA, 16 bp, Artificial Sequence
  01100101 Library A
gtagagtcagagtgac

SEQ ID NO: 696 - DNA, 16 bp, Artificial Sequence
  01100110 Library A
gtagagtcagtctgac

SEQ ID NO: 697 - DNA, 16 bp, Artificial Sequence
  01100111 Library A
gtagagtcagtgtgac

SEQ ID NO: 698 - DNA, 16 bp, Artificial Sequence
  01101000 Library A
gtagagtctcactgac

SEQ ID NO: 699 - DNA, 16 bp, Artificial Sequence
  01101001 Library A
gtagagtctcagtgac

SEQ ID NO: 700 - DNA, 16 bp, Artificial Sequence
  01101010 Library A
gtagagtctctctgac

SEQ ID NO: 701 - DNA, 16 bp, Artificial Sequence
  01101011 Library A
gtagagtctctgtgac

SEQ ID NO: 702 - DNA, 16 bp, Artificial Sequence
  01101100 Library A
gtagagtctgactgac

SEQ ID NO: 703 - DNA, 16 bp, Artificial Sequence
  01101101 Library A
gtagagtctgagtgac

SEQ ID NO: 704 - DNA, 16 bp, Artificial Sequence
  01101110 Library A
gtagagtctgtctgac

SEQ ID NO: 705 - DNA, 16 bp, Artificial Sequence
  01101111 Library A
gtagagtctgtgtgac

SEQ ID NO: 706 - DNA, 16 bp, Artificial Sequence
  01110000 Library A
gtagagtgacactgac

SEQ ID NO: 707 - DNA, 16 bp, Artificial Sequence
  01110001 Library A
gtagagtgacagtgac

SEQ ID NO: 708 - DNA, 16 bp, Artificial Sequence
  01110010 Library A
gtagagtgactctgac

SEQ ID NO: 709 - DNA, 16 bp, Artificial Sequence
  01110011 Library A
gtagagtgactgtgac

SEQ ID NO: 710 - DNA, 16 bp, Artificial Sequence
  01110100 Library A
gtagagtgagactgac

SEQ ID NO: 711 - DNA, 16 bp, Artificial Sequence
  01110101 Library A
gtagagtgagagtgac

SEQ ID NO: 712 - DNA, 16 bp, Artificial Sequence
  01110110 Library A
gtagagtgagtctgac

SEQ ID NO: 713 - DNA, 16 bp, Artificial Sequence
  01110111 Library A
gtagagtgagtgtgac

SEQ ID NO: 714 - DNA, 16 bp, Artificial Sequence
  01111000 Library A
gtagagtgtcactgac

SEQ ID NO: 715 - DNA, 16 bp, Artificial Sequence
  01111001 Library A
gtagagtgtcagtgac

SEQ ID NO: 716 - DNA, 16 bp, Artificial Sequence
  01111010 Library A
gtagagtgtctctgac

SEQ ID NO: 717 - DNA, 16 bp, Artificial Sequence
  01111011 Library A
gtagagtgtctgtgac

SEQ ID NO: 718 - DNA, 16 bp, Artificial Sequence
  01111100 Library A
gtagagtgtgactgac

SEQ ID NO: 719 - DNA, 16 bp, Artificial Sequence
  01111101 Library A
gtagagtgtgagtgac

SEQ ID NO: 720 - DNA, 16 bp, Artificial Sequence
  01111110 Library A
gtagagtgtgtctgac

SEQ ID NO: 721 - DNA, 16 bp, Artificial Sequence
  01111111 Library A
gtagagtgtgtgtgac

SEQ ID NO: 722 - DNA, 16 bp, Artificial Sequence
  10000000 Library A
gtagtcacacactgac

SEQ ID NO: 723 - DNA, 16 bp, Artificial Sequence
  10000001 Library A
gtagtcacacagtgac

SEQ ID NO: 724 - DNA, 16 bp, Artificial Sequence
  10000010 Library A
gtagtcacactctgac

SEQ ID NO: 725 - DNA, 16 bp, Artificial Sequence
  10000011 Library A
gtagtcacactgtgac

SEQ ID NO: 726 - DNA, 16 bp, Artificial Sequence
  10000100 Library A
gtagtcacagactgac

SEQ ID NO: 727 - DNA, 16 bp, Artificial Sequence
  10000101 Library A
gtagtcacagagtgac

SEQ ID NO: 728 - DNA, 16 bp, Artificial Sequence
  10000110 Library A
gtagtcacagtctgac

SEQ ID NO: 729 - DNA, 16 bp, Artificial Sequence
  10000111 Library A
gtagtcacagtgtgac

SEQ ID NO: 730 - DNA, 16 bp, Artificial Sequence
  10001000 Library A
gtagtcactcactgac

SEQ ID NO: 731 - DNA, 16 bp, Artificial Sequence
  10001001 Library A
gtagtcactcagtgac

SEQ ID NO: 732 - DNA, 16 bp, Artificial Sequence
  10001010 Library A
gtagtcactctctgac

SEQ ID NO: 733 - DNA, 16 bp, Artificial Sequence
  10001011 Library A
gtagtcactctgtgac

SEQ ID NO: 734 - DNA, 16 bp, Artificial Sequence
  10001100 Library A
gtagtcactgactgac

SEQ ID NO: 735 - DNA, 16 bp, Artificial Sequence
  10001101 Library A
gtagtcactgagtgac

SEQ ID NO: 736 - DNA, 16 bp, Artificial Sequence
  10001110 Library A
gtagtcactgtctgac

SEQ ID NO: 737 - DNA, 16 bp, Artificial Sequence
  10001111 Library A
gtagtcactgtgtgac

SEQ ID NO: 738 - DNA, 16 bp, Artificial Sequence
  10010000 Library A
gtagtcagacactgac

SEQ ID NO: 739 - DNA, 16 bp, Artificial Sequence
  10010001 Library A
gtagtcagacagtgac

SEQ ID NO: 740 - DNA, 16 bp, Artificial Sequence
  10010010 Library A
gtagtcagactctgac

SEQ ID NO: 741 - DNA, 16 bp, Artificial Sequence
  10010011 Library A
gtagtcagactgtgac

SEQ ID NO: 742 - DNA, 16 bp, Artificial Sequence
  10010100 Library A
gtagtcagagactgac

SEQ ID NO: 743 - DNA, 16 bp, Artificial Sequence
  10010101 Library A
gtagtcagagagtgac

SEQ ID NO: 744 - DNA, 16 bp, Artificial Sequence
  10010110 Library A
gtagtcagagtctgac

SEQ ID NO: 745 - DNA, 16 bp, Artificial Sequence
  10010111 Library A
gtagtcagagtgtgac

SEQ ID NO: 746 - DNA, 16 bp, Artificial Sequence
  10011000 Library A
gtagtcagtcactgac

SEQ ID NO: 747 - DNA, 16 bp, Artificial Sequence
  10011001 Library A
gtagtcagtcagtgac

SEQ ID NO: 748 - DNA, 16 bp, Artificial Sequence
  10011010 Library A
gtagtcagtctctgac

SEQ ID NO: 749 - DNA, 16 bp, Artificial Sequence
  10011011 Library A
gtagtcagtctgtgac

SEQ ID NO: 750 - DNA, 16 bp, Artificial Sequence
  10011100 Library A
gtagtcagtgactgac

SEQ ID NO: 751 - DNA, 16 bp, Artificial Sequence
  10011101 Library A
gtagtcagtgagtgac

SEQ ID NO: 752 - DNA, 16 bp, Artificial Sequence
  10011110 Library A
gtagtcagtgtctgac

SEQ ID NO: 753 - DNA, 16 bp, Artificial Sequence
  10011111 Library A
gtagtcagtgtgtgac

SEQ ID NO: 754 - DNA, 16 bp, Artificial Sequence
  10100000 Library A
gtagtctcacactgac

SEQ ID NO: 755 - DNA, 16 bp, Artificial Sequence
  10100001 Library A
gtagtctcacagtgac

SEQ ID NO: 756 - DNA, 16 bp, Artificial Sequence
  10100010 Library A
gtagtctcactctgac

SEQ ID NO: 757 - DNA, 16 bp, Artificial Sequence
  10100011 Library A
gtagtctcactgtgac

SEQ ID NO: 758 - DNA, 16 bp, Artificial Sequence
  10100100 Library A
gtagtctcagactgac

SEQ ID NO: 759 - DNA, 16 bp, Artificial Sequence
  10100101 Library A
gtagtctcagagtgac

SEQ ID NO: 760 - DNA, 16 bp, Artificial Sequence
  10100110 Library A
gtagtctcagtctgac

SEQ ID NO: 761 - DNA, 16 bp, Artificial Sequence
  10100111 Library A
gtagtctcagtgtgac

SEQ ID NO: 762 - DNA, 16 bp, Artificial Sequence
  10101000 Library A
gtagtctctcactgac

SEQ ID NO: 763 - DNA, 16 bp, Artificial Sequence
  10101001 Library A
gtagtctctcagtgac

SEQ ID NO: 764 - DNA, 16 bp, Artificial Sequence
  10101010 Library A
gtagtctctctctgac

SEQ ID NO: 765 - DNA, 16 bp, Artificial Sequence
  10101011 Library A
gtagtctctctgtgac

SEQ ID NO: 766 - DNA, 16 bp, Artificial Sequence
  10101100 Library A
gtagtctctgactgac

SEQ ID NO: 767 - DNA, 16 bp, Artificial Sequence
  10101101 Library A
gtagtctctgagtgac

SEQ ID NO: 768 - DNA, 16 bp, Artificial Sequence
  10101110 Library A
gtagtctctgtctgac

SEQ ID NO: 769 - DNA, 16 bp, Artificial Sequence
  10101111 Library A
gtagtctctgtgtgac

SEQ ID NO: 770 - DNA, 16 bp, Artificial Sequence
  10110000 Library A
gtagtctgacactgac

SEQ ID NO: 771 - DNA, 16 bp, Artificial Sequence
  10110001 Library A
gtagtctgacagtgac

SEQ ID NO: 772 - DNA, 16 bp, Artificial Sequence
  10110010 Library A
gtagtctgactctgac

SEQ ID NO: 773 - DNA, 16 bp, Artificial Sequence
  10110011 Library A
gtagtctgactgtgac

SEQ ID NO: 774 - DNA, 16 bp, Artificial Sequence
  10110100 Library A
gtagtctgagactgac

SEQ ID NO: 775 - DNA, 16 bp, Artificial Sequence
  10110101 Library A
gtagtctgagagtgac

SEQ ID NO: 776 - DNA, 16 bp, Artificial Sequence
  10110110 Library A
gtagtctgagtctgac

SEQ ID NO: 777 - DNA, 16 bp, Artificial Sequence
  10110111 Library A
gtagtctgagtgtgac

SEQ ID NO: 778 - DNA, 16 bp, Artificial Sequence
  10111000 Library A
gtagtctgtcactgac

SEQ ID NO: 779 - DNA, 16 bp, Artificial Sequence
  10111001 Library A
gtagtctgtcagtgac

SEQ ID NO: 780 - DNA, 16 bp, Artificial Sequence
  10111010 Library A
gtagtctgtctctgac

SEQ ID NO: 781 - DNA, 16 bp, Artificial Sequence
  10111011 Library A
gtagtctgtctgtgac

SEQ ID NO: 782 - DNA, 16 bp, Artificial Sequence
  10111100 Library A
gtagtctgtgactgac

SEQ ID NO: 783 - DNA, 16 bp, Artificial Sequence
  10111101 Library A
gtagtctgtgagtgac

SEQ ID NO: 784 - DNA, 16 bp, Artificial Sequence
  10111110 Library A
gtagtctgtgtctgac

SEQ ID NO: 785 - DNA, 16 bp, Artificial Sequence
  10111111 Library A
gtagtctgtgtgtgac

SEQ ID NO: 786 - DNA, 16 bp, Artificial Sequence
  11000000 Library A
gtagtgacacactgac

SEQ ID NO: 787 - DNA, 16 bp, Artificial Sequence
  11000001 Library A
gtagtgacacagtgac

SEQ ID NO: 788 - DNA, 16 bp, Artificial Sequence
  11000010 Library A
gtagtgacactctgac

SEQ ID NO: 789 - DNA, 16 bp, Artificial Sequence
  11000011 Library A
gtagtgacactgtgac

SEQ ID NO: 790 - DNA, 16 bp, Artificial Sequence
  11000100 Library A
gtagtgacagactgac

SEQ ID NO: 791 - DNA, 16 bp, Artificial Sequence
  11000101 Library A
gtagtgacagagtgac

SEQ ID NO: 792 - DNA, 16 bp, Artificial Sequence
  11000110 Library A
gtagtgacagtctgac

SEQ ID NO: 793 - DNA, 16 bp, Artificial Sequence
  11000111 Library A
gtagtgacagtgtgac

SEQ ID NO: 794 - DNA, 16 bp, Artificial Sequence
  11001000 Library A
gtagtgactcactgac

SEQ ID NO: 795 - DNA, 16 bp, Artificial Sequence
  11001001 Library A
gtagtgactcagtgac

SEQ ID NO: 796 - DNA, 16 bp, Artificial Sequence
  11001010 Library A
gtagtgactctctgac

SEQ ID NO: 797 - DNA, 16 bp, Artificial Sequence
  11001011 Library A
gtagtgactctgtgac

SEQ ID NO: 798 - DNA, 16 bp, Artificial Sequence
  11001100 Library A
gtagtgactgactgac

SEQ ID NO: 799 - DNA, 16 bp, Artificial Sequence
  11001101 Library A
gtagtgactgagtgac

SEQ ID NO: 800 - DNA, 16 bp, Artificial Sequence
  11001110 Library A
gtagtgactgtctgac

SEQ ID NO: 801 - DNA, 16 bp, Artificial Sequence
  11001111 Library A
gtagtgactgtgtgac

SEQ ID NO: 802 - DNA, 16 bp, Artificial Sequence
  11010000 Library A
gtagtgagacactgac

SEQ ID NO: 803 - DNA, 16 bp, Artificial Sequence
  11010001 Library A
gtagtgagacagtgac

SEQ ID NO: 804 - DNA, 16 bp, Artificial Sequence
  11010010 Library A
gtagtgagactctgac

SEQ ID NO: 805 - DNA, 16 bp, Artificial Sequence
  11010011 Library A
gtagtgagactgtgac

SEQ ID NO: 806 - DNA, 16 bp, Artificial Sequence
  11010100 Library A
gtagtgagagactgac

SEQ ID NO: 807 - DNA, 16 bp, Artificial Sequence
  11010101 Library A
gtagtgagagagtgac

SEQ ID NO: 808 - DNA, 16 bp, Artificial Sequence
  11010110 Library A
gtagtgagagtctgac

SEQ ID NO: 809 - DNA, 16 bp, Artificial Sequence
  11010111 Library A
gtagtgagagtgtgac

SEQ ID NO: 810 - DNA, 16 bp, Artificial Sequence
  11011000 Library A
gtagtgagtcactgac

SEQ ID NO: 811 - DNA, 16 bp, Artificial Sequence
  11011001 Library A
gtagtgagtcagtgac

SEQ ID NO: 812 - DNA, 16 bp, Artificial Sequence
  11011010 Library A
gtagtgagtctctgac

SEQ ID NO: 813 - DNA, 16 bp, Artificial Sequence
  11011011 Library A
gtagtgagtctgtgac

SEQ ID NO: 814 - DNA, 16 bp, Artificial Sequence
  11011100 Library A
gtagtgagtgactgac

SEQ ID NO: 815 - DNA, 16 bp, Artificial Sequence
  11011101 Library A
gtagtgagtgagtgac

SEQ ID NO: 816 - DNA, 16 bp, Artificial Sequence
  11011110 Library A
gtagtgagtgtctgac

SEQ ID NO: 817 - DNA, 16 bp, Artificial Sequence
  11011111 Library A
gtagtgagtgtgtgac

SEQ ID NO: 818 - DNA, 16 bp, Artificial Sequence
  11100000 Library A
gtagtgtcacactgac

SEQ ID NO: 819 - DNA, 16 bp, Artificial Sequence
  11100001 Library A
gtagtgtcacagtgac

SEQ ID NO: 820 - DNA, 16 bp, Artificial Sequence
  11100010 Library A
gtagtgtcactctgac

SEQ ID NO: 821 - DNA, 16 bp, Artificial Sequence
  11100011 Library A
gtagtgtcactgtgac

SEQ ID NO: 822 - DNA, 16 bp, Artificial Sequence
  11100100 Library A
gtagtgtcagactgac

SEQ ID NO: 823 - DNA, 16 bp, Artificial Sequence
  11100101 Library A
gtagtgtcagagtgac

SEQ ID NO: 824 - DNA, 16 bp, Artificial Sequence
  11100110 Library A
gtagtgtcagtctgac

SEQ ID NO: 825 - DNA, 16 bp, Artificial Sequence
  11100111 Library A
gtagtgtcagtgtgac

SEQ ID NO: 826 - DNA, 16 bp, Artificial Sequence
  11101000 Library A
gtagtgtctcactgac

SEQ ID NO: 827 - DNA, 16 bp, Artificial Sequence
  11101001 Library A
gtagtgtctcagtgac

SEQ ID NO: 828 - DNA, 16 bp, Artificial Sequence
  11101010 Library A
gtagtgtctctctgac

SEQ ID NO: 829 - DNA, 16 bp, Artificial Sequence
  11101011 Library A
gtagtgtctctgtgac

SEQ ID NO: 830 - DNA, 16 bp, Artificial Sequence
  11101100 Library A
gtagtgtctgactgac

SEQ ID NO: 831 - DNA, 16 bp, Artificial Sequence
  11101101 Library A
gtagtgtctgagtgac

SEQ ID NO: 832 - DNA, 16 bp, Artificial Sequence
  11101110 Library A
gtagtgtctgtctgac

SEQ ID NO: 833 - DNA, 16 bp, Artificial Sequence
  11101111 Library A
gtagtgtctgtgtgac

SEQ ID NO: 834 - DNA, 16 bp, Artificial Sequence
  11110000 Library A
gtagtgtgacactgac

SEQ ID NO: 835 - DNA, 16 bp, Artificial Sequence
  11110001 Library A
gtagtgtgacagtgac

SEQ ID NO: 836 - DNA, 16 bp, Artificial Sequence
  11110010 Library A
gtagtgtgactctgac

SEQ ID NO: 837 - DNA, 16 bp, Artificial Sequence
  11110011 Library A
gtagtgtgactgtgac

SEQ ID NO: 838 - DNA, 16 bp, Artificial Sequence
  11110100 Library A
gtagtgtgagactgac

SEQ ID NO: 839 - DNA, 16 bp, Artificial Sequence
  11110101 Library A
gtagtgtgagagtgac

SEQ ID NO: 840 - DNA, 16 bp, Artificial Sequence
  11110110 Library A
gtagtgtgagtctgac

SEQ ID NO: 841 - DNA, 16 bp, Artificial Sequence
  11110111 Library A
gtagtgtgagtgtgac

SEQ ID NO: 842 - DNA, 16 bp, Artificial Sequence
  11111000 Library A
gtagtgtgtcactgac

SEQ ID NO: 843 - DNA, 16 bp, Artificial Sequence
  11111001 Library A
gtagtgtgtcagtgac

SEQ ID NO: 844 - DNA, 16 bp, Artificial Sequence
  11111010 Library A
gtagtgtgtctctgac

SEQ ID NO: 845 - DNA, 16 bp, Artificial Sequence
  11111011 Library A
gtagtgtgtctgtgac

SEQ ID NO: 846 - DNA, 16 bp, Artificial Sequence
  11111100 Library A
gtagtgtgtgactgac

SEQ ID NO: 847 - DNA, 16 bp, Artificial Sequence
  11111101 Library A
gtagtgtgtgagtgac

SEQ ID NO: 848 - DNA, 16 bp, Artificial Sequence
  11111110 Library A
gtagtgtgtgtctgac

SEQ ID NO: 849 - DNA, 16 bp, Artificial Sequence
  11111111 Library A
gtagtgtgtgtgtgac

SEQ ID NO: 850 - DNA, 16 bp, Artificial Sequence
  00000000 Library B
tgacacacacactcag

SEQ ID NO: 851 - DNA, 16 bp, Artificial Sequence
  00000001 Library B
tgacacacacagtcag

SEQ ID NO: 852 - DNA, 16 bp, Artificial Sequence
  00000010 Library B
tgacacacactctcag

SEQ ID NO: 853 - DNA, 16 bp, Artificial Sequence
  00000011 Library B
tgacacacactgtcag

SEQ ID NO: 854 - DNA, 16 bp, Artificial Sequence
  00000100 Library B
tgacacacagactcag

SEQ ID NO: 855 - DNA, 16 bp, Artificial Sequence
  00000101 Library B
tgacacacagagtcag

SEQ ID NO: 856 - DNA, 16 bp, Artificial Sequence
  00000110 Library B
tgacacacagtctcag

SEQ ID NO: 857 - DNA, 16 bp, Artificial Sequence
  00000111 Library B
tgacacacagtgtcag

SEQ ID NO: 858 - DNA, 16 bp, Artificial Sequence
  00001000 Library B
tgacacactcactcag

SEQ ID NO: 859 - DNA, 16 bp, Artificial Sequence
  00001001 Library B
tgacacactcagtcag

SEQ ID NO: 860 - DNA, 16 bp, Artificial Sequence
  00001010 Library B
tgacacactctctcag

SEQ ID NO: 861 - DNA, 16 bp, Artificial Sequence
  00001011 Library B
tgacacactctgtcag

SEQ ID NO: 862 - DNA, 16 bp, Artificial Sequence
  00001100 Library B
tgacacactgactcag

SEQ ID NO: 863 - DNA, 16 bp, Artificial Sequence
  00001101 Library B
tgacacactgagtcag

SEQ ID NO: 864 - DNA, 16 bp, Artificial Sequence
  00001110 Library B
tgacacactgtctcag

SEQ ID NO: 865 - DNA, 16 bp, Artificial Sequence
  00001111 Library B
tgacacactgtgtcag

SEQ ID NO: 866 - DNA, 16 bp, Artificial Sequence
  00010000 Library B
tgacacagacactcag

SEQ ID NO: 867 - DNA, 16 bp, Artificial Sequence
  00010001 Library B
tgacacagacagtcag

SEQ ID NO: 868 - DNA, 16 bp, Artificial Sequence
  00010010 Library B
tgacacagactctcag

SEQ ID NO: 869 - DNA, 16 bp, Artificial Sequence
  00010011 Library B
tgacacagactgtcag

SEQ ID NO: 870 - DNA, 16 bp, Artificial Sequence
  00010100 Library B
tgacacagagactcag

SEQ ID NO: 871 - DNA, 16 bp, Artificial Sequence
  00010101 Library B
tgacacagagagtcag

SEQ ID NO: 872 - DNA, 16 bp, Artificial Sequence
  00010110 Library B
tgacacagagtctcag

SEQ ID NO: 873 - DNA, 16 bp, Artificial Sequence
  00010111 Library B
tgacacagagtgtcag

SEQ ID NO: 874 - DNA, 16 bp, Artificial Sequence
  00011000 Library B
tgacacagtcactcag

SEQ ID NO: 875 - DNA, 16 bp, Artificial Sequence
  00011001 Library B
tgacacagtcagtcag

SEQ ID NO: 876 - DNA, 16 bp, Artificial Sequence
  00011010 Library B
tgacacagtctctcag

SEQ ID NO: 877 - DNA, 16 bp, Artificial Sequence
  00011011 Library B
tgacacagtctgtcag

SEQ ID NO: 878 - DNA, 16 bp, Artificial Sequence
  00011100 Library B
tgacacagtgactcag

SEQ ID NO: 879 - DNA, 16 bp, Artificial Sequence
  00011101 Library B
tgacacagtgagtcag

SEQ ID NO: 880 - DNA, 16 bp, Artificial Sequence
  00011110 Library B
tgacacagtgtctcag

SEQ ID NO: 881 - DNA, 16 bp, Artificial Sequence
  00011111 Library B
tgacacagtgtgtcag

SEQ ID NO: 882 - DNA, 16 bp, Artificial Sequence
  00100000 Library B
tgacactcacactcag

SEQ ID NO: 883 - DNA, 16 bp, Artificial Sequence
  00100001 Library B
tgacactcacagtcag

SEQ ID NO: 884 - DNA, 16 bp, Artificial Sequence
  00100010 Library B
tgacactcactctcag

SEQ ID NO: 885 - DNA, 16 bp, Artificial Sequence
  00100011 Library B
tgacactcactgtcag

SEQ ID NO: 886 - DNA, 16 bp, Artificial Sequence
  00100100 Library B
tgacactcagactcag

SEQ ID NO: 887 - DNA, 16 bp, Artificial Sequence
  00100101 Library B
tgacactcagagtcag

SEQ ID NO: 888 - DNA, 16 bp, Artificial Sequence
  00100110 Library B
tgacactcagtctcag

SEQ ID NO: 889 - DNA, 16 bp, Artificial Sequence
  00100111 Library B
tgacactcagtgtcag

SEQ ID NO: 890 - DNA, 16 bp, Artificial Sequence
  00101000 Library B
tgacactctcactcag

SEQ ID NO: 891 - DNA, 16 bp, Artificial Sequence
  00101001 Library B
tgacactctcagtcag

SEQ ID NO: 892 - DNA, 16 bp, Artificial Sequence
  00101010 Library B
tgacactctctctcag

SEQ ID NO: 893 - DNA, 16 bp, Artificial Sequence
  00101011 Library B
tgacactctctgtcag

SEQ ID NO: 894 - DNA, 16 bp, Artificial Sequence
  00101100 Library B
tgacactctgactcag

SEQ ID NO: 895 - DNA, 16 bp, Artificial Sequence
  00101101 Library B
tgacactctgagtcag

SEQ ID NO: 896 - DNA, 16 bp, Artificial Sequence
  00101110 Library B
tgacactctgtctcag

SEQ ID NO: 897 - DNA, 16 bp, Artificial Sequence
  00101111 Library B
tgacactctgtgtcag

SEQ ID NO: 898 - DNA, 16 bp, Artificial Sequence
  00110000 Library B
tgacactgacactcag

SEQ ID NO: 899 - DNA, 16 bp, Artificial Sequence
  00110001 Library B
tgacactgacagtcag

SEQ ID NO: 900 - DNA, 16 bp, Artificial Sequence
  00110010 Library B
tgacactgactctcag

SEQ ID NO: 901 - DNA, 16 bp, Artificial Sequence
  00110011 Library B
tgacactgactgtcag

SEQ ID NO: 902 - DNA, 16 bp, Artificial Sequence
  00110100 Library B
tgacactgagactcag

SEQ ID NO: 903 - DNA, 16 bp, Artificial Sequence
  00110101 Library B
tgacactgagagtcag

SEQ ID NO: 904 - DNA, 16 bp, Artificial Sequence
  00110110 Library B
tgacactgagtctcag

SEQ ID NO: 905 - DNA, 16 bp, Artificial Sequence
  00110111 Library B
tgacactgagtgtcag

SEQ ID NO: 906 - DNA, 16 bp, Artificial Sequence
  00111000 Library B
tgacactgtcactcag

SEQ ID NO: 907 - DNA, 16 bp, Artificial Sequence
  00111001 Library B
tgacactgtcagtcag

SEQ ID NO: 908 - DNA, 16 bp, Artificial Sequence
  00111010 Library B
tgacactgtctctcag

SEQ ID NO: 909 - DNA, 16 bp, Artificial Sequence
  00111011 Library B
tgacactgtctgtcag

SEQ ID NO: 910 - DNA, 16 bp, Artificial Sequence
  00111100 Library B
tgacactgtgactcag

SEQ ID NO: 911 - DNA, 16 bp, Artificial Sequence
  00111101 Library B
tgacactgtgagtcag

SEQ ID NO: 912 - DNA, 16 bp, Artificial Sequence
  00111110 Library B
tgacactgtgtctcag

SEQ ID NO: 913 - DNA, 16 bp, Artificial Sequence
  00111111 Library B
tgacactgtgtgtcag

SEQ ID NO: 914 - DNA, 16 bp, Artificial Sequence
  01000000 Library B
tgacagacacactcag

SEQ ID NO: 915 - DNA, 16 bp, Artificial Sequence
  01000001 Library B
tgacagacacagtcag

SEQ ID NO: 916 - DNA, 16 bp, Artificial Sequence
  01000010 Library B
tgacagacactctcag

SEQ ID NO: 917 - DNA, 16 bp, Artificial Sequence
  01000011 Library B
tgacagacactgtcag

SEQ ID NO: 918 - DNA, 16 bp, Artificial Sequence
  01000100 Library B
tgacagacagactcag

SEQ ID NO: 919 - DNA, 16 bp, Artificial Sequence
  01000101 Library B
tgacagacagagtcag

SEQ ID NO: 920 - DNA, 16 bp, Artificial Sequence
  01000110 Library B
tgacagacagtctcag

SEQ ID NO: 921 - DNA, 16 bp, Artificial Sequence
  01000111 Library B
tgacagacagtgtcag

SEQ ID NO: 922 - DNA, 16 bp, Artificial Sequence
  01001000 Library B
tgacagactcactcag

SEQ ID NO: 923 - DNA, 16 bp, Artificial Sequence
  01001001 Library B
tgacagactcagtcag

SEQ ID NO: 924 - DNA, 16 bp, Artificial Sequence
  01001010 Library B
tgacagactctctcag

SEQ ID NO: 925 - DNA, 16 bp, Artificial Sequence
  01001011 Library B
tgacagactctgtcag

SEQ ID NO: 926 - DNA, 16 bp, Artificial Sequence
  01001100 Library B
tgacagactgactcag

SEQ ID NO: 927 - DNA, 16 bp, Artificial Sequence
  01001101 Library B
tgacagactgagtcag

SEQ ID NO: 928 - DNA, 16 bp, Artificial Sequence
  01001110 Library B
tgacagactgtctcag

SEQ ID NO: 929 - DNA, 16 bp, Artificial Sequence
  01001111 Library B
tgacagactgtgtcag

SEQ ID NO: 930 - DNA, 16 bp, Artificial Sequence
  01010000 Library B
tgacagagacactcag

SEQ ID NO: 931 - DNA, 16 bp, Artificial Sequence
  01010001 Library B
tgacagagacagtcag

SEQ ID NO: 932 - DNA, 16 bp, Artificial Sequence
  01010010 Library B
tgacagagactctcag

SEQ ID NO: 933 - DNA, 16 bp, Artificial Sequence
  01010011 Library B
tgacagagactgtcag

SEQ ID NO: 934 - DNA, 16 bp, Artificial Sequence
  01010100 Library B
tgacagagagactcag

SEQ ID NO: 935 - DNA, 16 bp, Artificial Sequence
  01010101 Library B
tgacagagagagtcag

SEQ ID NO: 936 - DNA, 16 bp, Artificial Sequence
  01010110 Library B
tgacagagagtctcag

SEQ ID NO: 937 - DNA, 16 bp, Artificial Sequence
  01010111 Library B
tgacagagagtgtcag

SEQ ID NO: 938 - DNA, 16 bp, Artificial Sequence
  01011000 Library B
tgacagagtcactcag

SEQ ID NO: 939 - DNA, 16 bp, Artificial Sequence
  01011001 Library B
tgacagagtcagtcag

SEQ ID NO: 940 - DNA, 16 bp, Artificial Sequence
  01011010 Library B
tgacagagtctctcag

SEQ ID NO: 941 - DNA, 16 bp, Artificial Sequence
  01011011 Library B
tgacagagtctgtcag

SEQ ID NO: 942 - DNA, 16 bp, Artificial Sequence
  01011100 Library B
tgacagagtgactcag

SEQ ID NO: 943 - DNA, 16 bp, Artificial Sequence
  01011101 Library B
tgacagagtgagtcag

SEQ ID NO: 944 - DNA, 16 bp, Artificial Sequence
  01011110 Library B
tgacagagtgtctcag

SEQ ID NO: 945 - DNA, 16 bp, Artificial Sequence
  01011111 Library B
tgacagagtgtgtcag

SEQ ID NO: 946 - DNA, 16 bp, Artificial Sequence
  01100000 Library B
tgacagtcacactcag

SEQ ID NO: 947 - DNA, 16 bp, Artificial Sequence
  01100001 Library B
tgacagtcacagtcag

SEQ ID NO: 948 - DNA, 16 bp, Artificial Sequence
  01100010 Library B
tgacagtcactctcag

SEQ ID NO: 949 - DNA, 16 bp, Artificial Sequence
  01100011 Library B
tgacagtcactgtcag

SEQ ID NO: 950 - DNA, 16 bp, Artificial Sequence
  01100100 Library B
tgacagtcagactcag

SEQ ID NO: 951 - DNA, 16 bp, Artificial Sequence
  01100101 Library B
tgacagtcagagtcag

SEQ ID NO: 952 - DNA, 16 bp, Artificial Sequence
  01100110 Library B
tgacagtcagtctcag

SEQ ID NO: 953 - DNA, 16 bp, Artificial Sequence
  01100111 Library B
tgacagtcagtgtcag

SEQ ID NO: 954 - DNA, 16 bp, Artificial Sequence
  01101000 Library B
tgacagtctcactcag

SEQ ID NO: 955 - DNA, 16 bp, Artificial Sequence
  01101001 Library B
tgacagtctcagtcag

SEQ ID NO: 956 - DNA, 16 bp, Artificial Sequence
  01101010 Library B
tgacagtctctctcag

SEQ ID NO: 957 - DNA, 16 bp, Artificial Sequence
  01101011 Library B
tgacagtctctgtcag

SEQ ID NO: 958 - DNA, 16 bp, Artificial Sequence
  01101100 Library B
tgacagtctgactcag

SEQ ID NO: 959 - DNA, 16 bp, Artificial Sequence
  01101101 Library B
tgacagtctgagtcag

SEQ ID NO: 960 - DNA, 16 bp, Artificial Sequence
  01101110 Library B
tgacagtctgtctcag

SEQ ID NO: 961 - DNA, 16 bp, Artificial Sequence
  01101111 Library B
tgacagtctgtgtcag

SEQ ID NO: 962 - DNA, 16 bp, Artificial Sequence
  01110000 Library B
tgacagtgacactcag

SEQ ID NO: 963 - DNA, 16 bp, Artificial Sequence
  01110001 Library B
tgacagtgacagtcag

SEQ ID NO: 964 - DNA, 16 bp, Artificial Sequence
  01110010 Library B
tgacagtgactctcag

SEQ ID NO: 965 - DNA, 16 bp, Artificial Sequence
  01110011 Library B
tgacagtgactgtcag

SEQ ID NO: 966 - DNA, 16 bp, Artificial Sequence
  01110100 Library B
tgacagtgagactcag

SEQ ID NO: 967 - DNA, 16 bp, Artificial Sequence
  01110101 Library B
tgacagtgagagtcag

SEQ ID NO: 968 - DNA, 16 bp, Artificial Sequence
  01110110 Library B
tgacagtgagtctcag

SEQ ID NO: 969 - DNA, 16 bp, Artificial Sequence
  01110111 Library B
tgacagtgagtgtcag

SEQ ID NO: 970 - DNA, 16 bp, Artificial Sequence
  01111000 Library B
tgacagtgtcactcag

SEQ ID NO: 971 - DNA, 16 bp, Artificial Sequence
  01111001 Library B
tgacagtgtcagtcag

SEQ ID NO: 972 - DNA, 16 bp, Artificial Sequence
  01111010 Library B
tgacagtgtctctcag

SEQ ID NO: 973 - DNA, 16 bp, Artificial Sequence
  01111011 Library B
tgacagtgtctgtcag

SEQ ID NO: 974 - DNA, 16 bp, Artificial Sequence
  01111100 Library B
tgacagtgtgactcag

SEQ ID NO: 975 - DNA, 16 bp, Artificial Sequence
  01111101 Library B
tgacagtgtgagtcag

SEQ ID NO: 976 - DNA, 16 bp, Artificial Sequence
  01111110 Library B
tgacagtgtgtctcag

SEQ ID NO: 977 - DNA, 16 bp, Artificial Sequence
  01111111 Library B
tgacagtgtgtgtcag

SEQ ID NO: 978 - DNA, 16 bp, Artificial Sequence
  10000000 Library B
tgactcacacactcag

SEQ ID NO: 979 - DNA, 16 bp, Artificial Sequence
  10000001 Library B
tgactcacacagtcag

SEQ ID NO: 980 - DNA, 16 bp, Artificial Sequence
  10000010 Library B
tgactcacactctcag

SEQ ID NO: 981 - DNA, 16 bp, Artificial Sequence
  10000011 Library B
tgactcacactgtcag

SEQ ID NO: 982 - DNA, 16 bp, Artificial Sequence
  10000100 Library B
tgactcacagactcag

SEQ ID NO: 983 - DNA, 16 bp, Artificial Sequence
  10000101 Library B
tgactcacagagtcag

SEQ ID NO: 984 - DNA, 16 bp, Artificial Sequence
  10000110 Library B
tgactcacagtctcag

SEQ ID NO: 985 - DNA, 16 bp, Artificial Sequence
  10000111 Library B
tgactcacagtgtcag

SEQ ID NO: 986 - DNA, 16 bp, Artificial Sequence
  10001000 Library B
tgactcactcactcag

SEQ ID NO: 987 - DNA, 16 bp, Artificial Sequence
  10001001 Library B
tgactcactcagtcag

SEQ ID NO: 988 - DNA, 16 bp, Artificial Sequence
  10001010 Library B
tgactcactctctcag

SEQ ID NO: 989 - DNA, 16 bp, Artificial Sequence
  10001011 Library B
tgactcactctgtcag

SEQ ID NO: 990 - DNA, 16 bp, Artificial Sequence
  10001100 Library B
tgactcactgactcag

SEQ ID NO: 991 - DNA, 16 bp, Artificial Sequence
  10001101 Library B
tgactcactgagtcag

SEQ ID NO: 992 - DNA, 16 bp, Artificial Sequence
  10001110 Library B
tgactcactgtctcag

SEQ ID NO: 993 - DNA, 16 bp, Artificial Sequence
  10001111 Library B
tgactcactgtgtcag

SEQ ID NO: 994 - DNA, 16 bp, Artificial Sequence
  10010000 Library B
tgactcagacactcag

SEQ ID NO: 995 - DNA, 16 bp, Artificial Sequence
  10010001 Library B
tgactcagacagtcag

SEQ ID NO: 996 - DNA, 16 bp, Artificial Sequence
  10010010 Library B
tgactcagactctcag

SEQ ID NO: 997 - DNA, 16 bp, Artificial Sequence
  10010011 Library B
tgactcagactgtcag

SEQ ID NO: 998 - DNA, 16 bp, Artificial Sequence
  10010100 Library B
tgactcagagactcag

SEQ ID NO: 999 - DNA, 16 bp, Artificial Sequence
  10010101 Library B
tgactcagagagtcag

SEQ ID NO: 1000 - DNA, 16 bp, Artificial Sequence
  10010110 Library B
tgactcagagtctcag

SEQ ID NO: 1001 - DNA, 16 bp, Artificial Sequence
  10010111 Library B
tgactcagagtgtcag

SEQ ID NO: 1002 - DNA, 16 bp, Artificial Sequence
  10011000 Library B
tgactcagtcactcag

SEQ ID NO: 1003 - DNA, 16 bp, Artificial Sequence
  10011001 Library B
tgactcagtcagtcag

SEQ ID NO: 1004 - DNA, 16 bp, Artificial Sequence
  10011010 Library B
tgactcagtctctcag

SEQ ID NO: 1005 - DNA, 16 bp, Artificial Sequence
  10011011 Library B
tgactcagtctgtcag

SEQ ID NO: 1006 - DNA, 16 bp, Artificial Sequence
  10011100 Library B
tgactcagtgactcag

SEQ ID NO: 1007 - DNA, 16 bp, Artificial Sequence
  10011101 Library B
tgactcagtgagtcag

SEQ ID NO: 1008 - DNA, 16 bp, Artificial Sequence
  10011110 Library B
tgactcagtgtctcag

SEQ ID NO: 1009 - DNA, 16 bp, Artificial Sequence
  10011111 Library B
tgactcagtgtgtcag

SEQ ID NO: 1010 - DNA, 16 bp, Artificial Sequence
  10100000 Library B
tgactctcacactcag

SEQ ID NO: 1011 - DNA, 16 bp, Artificial Sequence
  10100001 Library B
tgactctcacagtcag

SEQ ID NO: 1012 - DNA, 16 bp, Artificial Sequence
  10100010 Library B
tgactctcactctcag

SEQ ID NO: 1013 - DNA, 16 bp, Artificial Sequence
  10100011 Library B
tgactctcactgtcag

SEQ ID NO: 1014 - DNA, 16 bp, Artificial Sequence
  10100100 Library B
tgactctcagactcag

SEQ ID NO: 1015 - DNA, 16 bp, Artificial Sequence
  10100101 Library B
tgactctcagagtcag

SEQ ID NO: 1016 - DNA, 16 bp, Artificial Sequence
  10100110 Library B
tgactctcagtctcag

SEQ ID NO: 1017 - DNA, 16 bp, Artificial Sequence
  10100111 Library B
tgactctcagtgtcag

SEQ ID NO: 1018 - DNA, 16 bp, Artificial Sequence
  10101000 Library B
tgactctctcactcag

SEQ ID NO: 1019 - DNA, 16 bp, Artificial Sequence
  10101001 Library B
tgactctctcagtcag

SEQ ID NO: 1020 - DNA, 16 bp, Artificial Sequence
  10101010 Library B
tgactctctctctcag

SEQ ID NO: 1021 - DNA, 16 bp, Artificial Sequence
  10101011 Library B
tgactctctctgtcag

SEQ ID NO: 1022 - DNA, 16 bp, Artificial Sequence
  10101100 Library B
tgactctctgactcag

SEQ ID NO: 1023 - DNA, 16 bp, Artificial Sequence
  10101101 Library B
tgactctctgagtcag

SEQ ID NO: 1024 - DNA, 16 bp, Artificial Sequence
  10101110 Library B
tgactctctgtctcag

SEQ ID NO: 1025 - DNA, 16 bp, Artificial Sequence
  10101111 Library B
tgactctctgtgtcag

SEQ ID NO: 1026 - DNA, 16 bp, Artificial Sequence
  10110000 Library B
tgactctgacactcag

SEQ ID NO: 1027 - DNA, 16 bp, Artificial Sequence
  10110001 Library B
tgactctgacagtcag

SEQ ID NO: 1028 - DNA, 16 bp, Artificial Sequence
  10110010 Library B
tgactctgactctcag

SEQ ID NO: 1029 - DNA, 16 bp, Artificial Sequence
  10110011 Library B
tgactctgactgtcag

SEQ ID NO: 1030 - DNA, 16 bp, Artificial Sequence
  10110100 Library B
tgactctgagactcag

SEQ ID NO: 1031 - DNA, 16 bp, Artificial Sequence
  10110101 Library B
tgactctgagagtcag

SEQ ID NO: 1032 - DNA, 16 bp, Artificial Sequence
  10110110 Library B
tgactctgagtctcag

SEQ ID NO: 1033 - DNA, 16 bp, Artificial Sequence
  10110111 Library B
tgactctgagtgtcag

SEQ ID NO: 1034 - DNA, 16 bp, Artificial Sequence
  10111000 Library B
tgactctgtcactcag

SEQ ID NO: 1035 - DNA, 16 bp, Artificial Sequence
  10111001 Library B
tgactctgtcagtcag

SEQ ID NO: 1036 - DNA, 16 bp, Artificial Sequence
  10111010 Library B
tgactctgtctctcag

SEQ ID NO: 1037 - DNA, 16 bp, Artificial Sequence
  10111011 Library B
tgactctgtctgtcag

SEQ ID NO: 1038 - DNA, 16 bp, Artificial Sequence
  10111100 Library B
tgactctgtgactcag

SEQ ID NO: 1039 - DNA, 16 bp, Artificial Sequence
  10111101 Library B
tgactctgtgagtcag

SEQ ID NO: 1040 - DNA, 16 bp, Artificial Sequence
  10111110 Library B
tgactctgtgtctcag

SEQ ID NO: 1041 - DNA, 16 bp, Artificial Sequence
  10111111 Library B
tgactctgtgtgtcag

SEQ ID NO: 1042 - DNA, 16 bp, Artificial Sequence
  11000000 Library B
tgactgacacactcag

SEQ ID NO: 1043 - DNA, 16 bp, Artificial Sequence
  11000001 Library B
tgactgacacagtcag

SEQ ID NO: 1044 - DNA, 16 bp, Artificial Sequence
  11000010 Library B
tgactgacactctcag

SEQ ID NO: 1045 - DNA, 16 bp, Artificial Sequence
  11000011 Library B
tgactgacactgtcag

SEQ ID NO: 1046 - DNA, 16 bp, Artificial Sequence
  11000100 Library B
tgactgacagactcag

SEQ ID NO: 1047 - DNA, 16 bp, Artificial Sequence
  11000101 Library B
tgactgacagagtcag

SEQ ID NO: 1048 - DNA, 16 bp, Artificial Sequence
  11000110 Library B
tgactgacagtctcag

SEQ ID NO: 1049 - DNA, 16 bp, Artificial Sequence
  11000111 Library B
tgactgacagtgtcag

SEQ ID NO: 1050 - DNA, 16 bp, Artificial Sequence
  11001000 Library B
tgactgactcactcag

SEQ ID NO: 1051 - DNA, 16 bp, Artificial Sequence
  11001001 Library B
tgactgactcagtcag

SEQ ID NO: 1052 - DNA, 16 bp, Artificial Sequence
  11001010 Library B
tgactgactctctcag

SEQ ID NO: 1053 - DNA, 16 bp, Artificial Sequence
  11001011 Library B
tgactgactctgtcag

SEQ ID NO: 1054 - DNA, 16 bp, Artificial Sequence
  11001100 Library B
tgactgactgactcag

SEQ ID NO: 1055 - DNA, 16 bp, Artificial Sequence
  11001101 Library B
tgactgactgagtcag

SEQ ID NO: 1056 - DNA, 16 bp, Artificial Sequence
  11001110 Library B
tgactgactgtctcag

SEQ ID NO: 1057 - DNA, 16 bp, Artificial Sequence
  11001111 Library B
tgactgactgtgtcag

SEQ ID NO: 1058 - DNA, 16 bp, Artificial Sequence
  11010000 Library B
tgactgagacactcag

SEQ ID NO: 1059 - DNA, 16 bp, Artificial Sequence
  11010001 Library B
tgactgagacagtcag

SEQ ID NO: 1060 - DNA, 16 bp, Artificial Sequence
  11010010 Library B
tgactgagactctcag

SEQ ID NO: 1061 - DNA, 16 bp, Artificial Sequence
  11010011 Library B
tgactgagactgtcag

SEQ ID NO: 1062 - DNA, 16 bp, Artificial Sequence
  11010100 Library B
tgactgagagactcag

SEQ ID NO: 1063 - DNA, 16 bp, Artificial Sequence
  11010101 Library B
tgactgagagagtcag

SEQ ID NO: 1064 - DNA, 16 bp, Artificial Sequence
  11010110 Library B
tgactgagagtctcag

SEQ ID NO: 1065 - DNA, 16 bp, Artificial Sequence
  11010111 Library B
tgactgagagtgtcag

SEQ ID NO: 1066 - DNA, 16 bp, Artificial Sequence
  11011000 Library B
tgactgagtcactcag

SEQ ID NO: 1067 - DNA, 16 bp, Artificial Sequence
  11011001 Library B
tgactgagtcagtcag

SEQ ID NO: 1068 - DNA, 16 bp, Artificial Sequence
  11011010 Library B
tgactgagtctctcag

SEQ ID NO: 1069 - DNA, 16 bp, Artificial Sequence
  11011011 Library B
tgactgagtctgtcag

SEQ ID NO: 1070 - DNA, 16 bp, Artificial Sequence
  11011100 Library B
tgactgagtgactcag

SEQ ID NO: 1071 - DNA, 16 bp, Artificial Sequence
  11011101 Library B
tgactgagtgagtcag

SEQ ID NO: 1072 - DNA, 16 bp, Artificial Sequence
  11011110 Library B
tgactgagtgtctcag

SEQ ID NO: 1073 - DNA, 16 bp, Artificial Sequence
  11011111 Library B
tgactgagtgtgtcag

SEQ ID NO: 1074 - DNA, 16 bp, Artificial Sequence
  11100000 Library B
tgactgtcacactcag

SEQ ID NO: 1075 - DNA, 16 bp, Artificial Sequence
  11100001 Library B
tgactgtcacagtcag

SEQ ID NO: 1076 - DNA, 16 bp, Artificial Sequence
  11100010 Library B
tgactgtcactctcag

SEQ ID NO: 1077 - DNA, 16 bp, Artificial Sequence
  11100011 Library B
tgactgtcactgtcag

SEQ ID NO: 1078 - DNA, 16 bp, Artificial Sequence
  11100100 Library B
tgactgtcagactcag

SEQ ID NO: 1079 - DNA, 16 bp, Artificial Sequence
  11100101 Library B
tgactgtcagagtcag

SEQ ID NO: 1080 - DNA, 16 bp, Artificial Sequence
  11100110 Library B
tgactgtcagtctcag

SEQ ID NO: 1081 - DNA, 16 bp, Artificial Sequence
  11100111 Library B
tgactgtcagtgtcag

SEQ ID NO: 1082 - DNA, 16 bp, Artificial Sequence
  11101000 Library B
tgactgtctcactcag

SEQ ID NO: 1083 - DNA, 16 bp, Artificial Sequence
  11101001 Library B
tgactgtctcagtcag

SEQ ID NO: 1084 - DNA, 16 bp, Artificial Sequence
  11101010 Library B
tgactgtctctctcag

SEQ ID NO: 1085 - DNA, 16 bp, Artificial Sequence
  11101011 Library B
tgactgtctctgtcag

SEQ ID NO: 1086 - DNA, 16 bp, Artificial Sequence
  11101100 Library B
tgactgtctgactcag

SEQ ID NO: 1087 - DNA, 16 bp, Artificial Sequence
  11101101 Library B
tgactgtctgagtcag

SEQ ID NO: 1088 - DNA, 16 bp, Artificial Sequence
  11101110 Library B
tgactgtctgtctcag

SEQ ID NO: 1089 - DNA, 16 bp, Artificial Sequence
  11101111 Library B
tgactgtctgtgtcag

SEQ ID NO: 1090 - DNA, 16 bp, Artificial Sequence
  11110000 Library B
tgactgtgacactcag

SEQ ID NO: 1091 - DNA, 16 bp, Artificial Sequence
  11110001 Library B
tgactgtgacagtcag

SEQ ID NO: 1092 - DNA, 16 bp, Artificial Sequence
  11110010 Library B
tgactgtgactctcag

SEQ ID NO: 1093 - DNA, 16 bp, Artificial Sequence
  11110011 Library B
tgactgtgactgtcag

SEQ ID NO: 1094 - DNA, 16 bp, Artificial Sequence
  11110100 Library B
tgactgtgagactcag

SEQ ID NO: 1095 - DNA, 16 bp, Artificial Sequence
  11110101 Library B
tgactgtgagagtcag

SEQ ID NO: 1096 - DNA, 16 bp, Artificial Sequence
  11110110 Library B
tgactgtgagtctcag

SEQ ID NO: 1097 - DNA, 16 bp, Artificial Sequence
  11110111 Library B
tgactgtgagtgtcag

SEQ ID NO: 1098 - DNA, 16 bp, Artificial Sequence
  11111000 Library B
tgactgtgtcactcag

SEQ ID NO: 1099 - DNA, 16 bp, Artificial Sequence
  11111001 Library B
tgactgtgtcagtcag

SEQ ID NO: 1100 - DNA, 16 bp, Artificial Sequence
  11111010 Library B
tgactgtgtctctcag

SEQ ID NO: 1101 - DNA, 16 bp, Artificial Sequence
  11111011 Library B
tgactgtgtctgtcag

SEQ ID NO: 1102 - DNA, 16 bp, Artificial Sequence
  11111100 Library B
tgactgtgtgactcag

SEQ ID NO: 1103 - DNA, 16 bp, Artificial Sequence
  11111101 Library B
tgactgtgtgagtcag

SEQ ID NO: 1104 - DNA, 16 bp, Artificial Sequence
  11111110 Library B
tgactgtgtgtctcag

SEQ ID NO: 1105 - DNA, 16 bp, Artificial Sequence
  11111111 Library B
tgactgtgtgtgtcag

SEQ ID NO: 1106 - DNA, 16 bp, Artificial Sequence
  EOF_B
tgaccagtctgttcag

SEQ ID NO: 1107 - DNA, 10 bp, Artificial Sequence
  Barecode 0
acacacacac